(19) United States
(12) Patent Application Publication
(10) Pub. No.: US 2024/0350488 A1
(43) Pub. Date: Oct. 24, 2024

(54) METHODS OF TREATING SWI/SNF DEREGULATED CANCERS

(71) Applicant: Fred Hutchinson Cancer Center, Seattle, WA (US)

(72) Inventors: Andrew Hsieh, Shoreline, WA (US); Sujata Jana, Seattle, WA (US)

(73) Assignee: Fred Hutchinson Cancer Center, Seattle, WA (US)

(21) Appl. No.: 18/639,526

(22) Filed: Apr. 18, 2024

Related U.S. Application Data
(60) Provisional application No. 63/496,926, filed on Apr. 18, 2023.

(51) Int. Cl. A61K 31/5025; A61K 31/138; A61K 45/06; A61P 35/00

(52) U.S. Cl. CPC A61K 31/5025; A61K 31/138; A61K 45/06; A61P 35/00

(57) ABSTRACT

The present disclosure provides therapeutic treatments for a disease such as a cancer or a tumor, including methods for treating SWI/SNF chromatin remodeling complex mutant tumors or cancers in a subject in need thereof. In an embodiment, the method comprises administering to the subject a therapeutically effective amount of at least one agent effective in suppressing or inhibiting mRNA translation. In an embodiment, the cancer or tumor is an ARID1A mutant and/or an ARID1A-deficient cancer or tumor. In some embodiments, the therapeutic treatments disclosed include methods of treating ARID1A mutant and/or ARID1A-deficient cancer in a subject comprising the step of administering a therapeutically effective amount of an agent effective in inhibiting mRNA translation elongation. In some embodiments, the agent effective in inhibiting mRNA translation elongation is an agent effective in suppressing or inhibiting eukaryotic elongation factor 2 (eEF2).

### CROSS-REFERENCE TO RELATED APPLICATION

This application claims the benefit of U.S. Provisional Patent Application No. 63/496,926, filed Apr. 18, 2023, which is hereby incorporated by reference.

### STATEMENT OF GOVERNMENT LICENSE RIGHTS

This invention was made with government support under W81XWH-19-1-0658 awarded by the Medical Research and Development Command, and CA230617, CA015704, GM135362, and CA276308 awarded by the National Institutes of Health. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 1896-P85US_Seq_List_20240417.xml. The XML file is 18,666 bytes; was created on Apr. 17, 2024; and is being submitted electronically via Patent Center with the filing of the specification.

### BACKGROUND

Major tumor suppressors such as TP53, PTEN, APC, and PDCD4 are negative regulators of mRNA translation. Downregulation or loss of tumor suppressor genes leads to an increase in mRNA-specific translation through cap-dependent and independent mechanisms enabling cancer pathogenesis. Recently, pan-cancer genome sequencing coupled with mechanistic work have uncovered a new class of tumor suppressors involved in chromatin remodeling. The ATP-dependent SWI/SNF (switch/sucrose non-fermentable) chromatin remodeling complex is made up of 15 subunits which regulate gene transcription by altering chromatin structure through hydrolysis of adenosine triphosphate (ATP) and are mutated in 20% of human cancers. Of the 15 subunits, the AT-rich interactive domain-containing protein IA (ARID1A), subunit of the SWI/SN F complex is commonly mutated in human cancers. ARID1A is considered a tumor suppressor because genetic deletion leads to the transcriptional activation of oncogenic mRNAs, impairments in double stranded DNA break repair and decatenation, and tumor progression. There exists an ongoing need for improved methods, kits, and compositions for treatment of ARID1A-mutant cancers or cancers with mutations to the SWI/SNF chromatin modeling complex.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, provided herein is a method for treating SWI/SNF chromatin remodeling complex mutant tumors or cancers in a subject in need thereof. In an embodiment, the method comprises administering to the subject a therapeutically effective amount of at least one agent effective in suppressing or inhibiting mRNA translation. Any agent capable of slowing, interfering, inhibiting, reducing, or suppressing mRNA translation can be used in the methods disclosed herein. In an embodiment, the tumors or cancers comprise ARID1A-mutant and/or ARID1A deficient tumors or cancers. In an aspect of the present disclosure, the method comprises administering to the subject a therapeutically effective amount of an agent effective in preventing, suppressing or inhibiting mRNA translation elongation. Any agent capable of slowing, interfering, inhibiting, reducing, preventing, or suppressing mRNA translation elongation can be used in the methods disclosed herein. In some embodiments, the agent comprises an agent effective in suppressing or inhibiting eukaryotic elongation factor 2 (eEF2). Any agent capable of slowing, interfering, inhibiting, reducing, or suppressing the activity or expression of eEF2 can be used in the methods disclosed herein. The method can further comprise administering to the subject a therapeutically effective amount of at least one additional cancer therapy. In some embodiments, the at least one additional therapy comprises a chemotherapeutic, target specific therapy, or immunotherapeutic agent. In some embodiments, the at least one additional therapy comprises radiation.

In some embodiments, the agent effective in inhibiting mRNA translation elongation comprises an agent effective in preventing, inhibiting or suppressing eEF2 activity. In some embodiments, the agent effective in inhibiting or suppressing eEF2 activity comprises an agent capable of activating eEF2 kinase (eEF2K). In some embodiments, the agent is capable of increasing eEF2K levels at the mRNA and/or protein levels. In some embodiments, the agent is capable of activating eEF2K by decreasing phosphorylation of eEF2K at serine 366 residue of the eEF2K protein and/or or increasing phosphorylation of eEF2K at S398.

In some embodiments, the agent effective in preventing, suppressing or inhibiting eEF2 activity comprise an agent effective in suppressing or inhibiting the MAPK pathway. In some embodiments, the agent effective in suppressing or inhibiting eEF2 activity comprises inhibitors of RASGRP1. In various embodiments, an agent effective in suppressing or inhibiting RASGRP1 comprises any agent capable of slowing, interfering, inhibiting, reducing, or suppressing the activity or expression of RASGRP1. Preferably, the agent effective in suppressing or inhibiting RASGRP1 comprises an agent effective in suppressing or inhibiting RASGRP1 transcript and/or protein.

In various embodiments, the agents that prevent or inhibit the activity of eEF2 can work by any known mechanism. Agents that inhibit eEF2 complex formation, eEF2 activity, or eEF2-dependent mRNA translation can be used in the methods disclosed herein. In certain embodiments of the methods disclosed herein, the agent that inhibits the activity of the eEF2 translation elongation factor is an agent that blocks binding of eEF2 to mRNA. In some embodiments, suitable agents include peptide mimetics of eEF2. In some embodiments, two or more agents that inhibit the activity of eEF2 can be used in the methods disclosed herein.

In various embodiments, the agents disclosed herein are individually a small molecule, protein, fusion protein, peptide, nucleic acid, aptamer, avimer, or derivatives or fragments thereof.

In some embodiments, the method comprises administering to the subject a therapeutically effective amount of an agent comprising a protein synthesis inhibitor. In some embodiments, the protein synthesis inhibitor is capable of suppressing or inhibiting mRNA translation. In some embodiments, the protein synthesis inhibitor is selected from inhibitors targeting eEF1A, eEF1B, EFsec, eEF2, eIF5A, and eEF3. In an exemplary embodiment, the protein synthesis inhibitor inhibits eEF1A. Exemplary inhibitors include but are not limited to SR-A3 and Ternatin-4. In some embodiments, the protein synthesis inhibitor comprises agents that inhibit eIF4E, eIF4A, eIF4G, eIF2 complex function, or eIF3 complex function. Exemplary agents include but are not limited to homoharringtonine (omacetaxine mepesuccinate), eFT508, eFT226, 4-[(3E)-3-[[5-(4-nitrophenyl)furan-2-yl]methylidene]-2-oxo-5-phenylpyrrol-1-yl]benzoic acid (4E1RCat), 5-[5-[(E)-(3-Benzyl-4-oxo-2-sulfanylidene-1,3-thiazolidin-5-ylidene)methyl]furan-2-yl]-2-chlorobenzoic acid (4E2RCat), α-[2-[4-(3,4-Dichlorophenyl)-2-thiazolyl]-hydrazin ylidene]-2-nitrobenzene-propanoic acid (4EGI-1), silvesterol, hippuristanol, rocaglates, pateamine A, elatol, sanguinarine, elisabatin A, 15d-PGJ2, their analogs, and combinations thereof. In some embodiments, the protein synthesis inhibitor inhibits the initiation of translation elongation. In some embodiments, the agent effective in preventing, inhibiting the initiation of mRNA translation elongation comprises an agent effective in inhibiting or suppressing translation elongation.

In various embodiments of the methods disclosed herein, the SWI/SNF chromatin remodeling complex mutant or ARID1A-deficient and/or ARID1A mutant tumor or cancer is selected from a cancer of the brain, breast, bladder, bone, cartilage, cervix, colon, neural tissue, glia, esophagus, fallopian tube, pancreas, intestines, gallbladder, kidney, liver, lung, ovary, pancreas, parathyroid, pineal gland, pituitary gland, prostate, spinal cord, spleen, skeletal muscle, skin, muscle, stomach, testis, thymus, thyroid, urogenital tract, ureter, urethra, uterus, endometrium, vagina, or combination thereof. In some embodiments, the SWI/SNF chromatin remodeling complex mutant or ARID1A-deficient and/or ARID1A-mutant tumor, or cancer is a urothelial cancer.

In various embodiments, the above method further comprises determining the presence of SWI/SNF chromatin remodeling complex mutation in the tumor or cancer cells of the subject prior to administering the agent. In some embodiments, the method comprises determining the presence of an ARID1A mutation and/or deficiency in the tumor or cancers cells of the subject prior to administering the agent. In various embodiments of the above method, the step of determining the presence of SWI/SNF chromatin remodeling complex mutation or ARID1A-mutation and/or ARID1A deficiency in the tumor or cancer cells of the subject and the step of administering the agent to the subject occur simultaneously or concurrently.

In another aspect, provided herein is a method of selecting and/or deselecting a subject having a tumor or cancer for treatment with a therapeutic agent, the method comprising: (i) obtaining a biological specimen from the tumor or cancer of the subject; (ii) determining the presence or absence of a SWI/SNF chromatin remodeling complex mutation or an ARID1A-mutation and/or ARID1A deficiency in the biological specimen obtained from the subject; and (iii) administering an effective amount of the therapeutic agent to the subject determined to have a presence of a SWI/SNF chromatin remodeling complex mutation or an ARID1A-deficiency. In some embodiments, the ARID1A-mutation and/or ARID1A deficiency comprises a low expression of ARID1A in the biological sample obtained from the tumor or cancer of the subject as compared to the expression of ARID1A in a corresponding biological sample obtained from a healthy subject.

In some embodiments, the therapeutic agent comprises an agent effective in suppressing or inhibiting mRNA translation elongation. In some embodiments, the therapeutic agent comprises an agent effective in preventing, suppressing or inhibiting eukaryotic elongation factor 2 (eEF2). Any agent capable of slowing, interfering, inhibiting, reducing, preventing, or suppressing the activity or expression of eEF2 can be used in the methods disclosed herein. In some embodiments, the method further comprises administering to the subject a therapeutically effective amount of at least one additional cancer therapy. In a particular embodiment, the at least one additional cancer therapy comprises administering an effective amount of a chemotherapeutic agent, target specific therapy, or immunotherapeutic agent or radiotherapy. The biological sample obtained from the subject may be a fluid sample, such as blood, serum plasma, ascites, sputum, saliva, or urine. The biological sample obtained from the subject may be a tissue sample, such as a cancer, pre-cancer or benign tumor tissue sample.

In some embodiments, the therapeutic agent effective in preventing, suppressing, or inhibiting mRNA translation elongation comprises an agent effective in inhibiting or suppressing eEF2 activity. In some embodiments, the therapeutic agent effective in inhibiting or suppressing eEF2 activity comprises an agent capable of activating eEF2 kinase (eEF2K). In some embodiments, the therapeutic agent is capable of increasing eEF2K levels at the mRNA and/or protein levels. In some embodiments, the therapeutic agent activates eEF2K by decreasing phosphorylation of eEF2K at serine 366 residue of the eEF2K protein and/or or increasing phosphorylation of eEF2K at S398.

In some embodiments, the therapeutic agent effective in preventing, suppressing or inhibiting eEF2 activity comprise an agent effective in suppressing or inhibiting the MAPK pathway. In some embodiments, the therapeutic agent effective in suppressing or inhibiting eEF2 activity comprises inhibitors of RASGRP1. In various embodiments, an agent effective in suppressing or inhibiting RASGRP1 comprises any agent capable of slowing, interfering, inhibiting, reducing, or suppressing the activity or expression of RASGRP1. Preferably, the therapeutic agent effective in suppressing or inhibiting RASGRP1 comprises an agent effective in suppressing or inhibiting RASGRP1 transcript and/or protein.

In various embodiments, the agents or therapeutic agents disclosed herein are individually a small molecule, a protein, a fusion protein, a peptide, a nucleic acid, an aptamer, an avimer, or derivatives or fragments thereof.

In some embodiments, the therapeutic agent comprises a protein synthesis inhibitor. In some embodiments, the protein synthesis inhibitor is capable of suppressing or inhibiting mRNA translation. In some embodiments, the protein synthesis inhibitor is selected from inhibitors targeting eEF1A, eEF1B, EFsec, eEF2, eIF5A, and eEF3. In an exemplary embodiment, the protein synthesis inhibitor inhibits eEF1A. Other suitable inhibitors include but are not limited to SR-A3 and Ternatin-4. In some embodiments, the protein synthesis inhibitor comprises agents that inhibit eIF4E, eIF4A, eIF4G, eIF2 complex function, or eIF3 complex function. Exemplary compounds include but are not limited to homoharringtonine (omacetaxine mepesuccinate), eFT508, eFT226, 4-[(3E)-3-[[5-(4-nitrophenyl)furan-2-yl]methylidene]-2-oxo-5-phenylpyrrol-1-yl]benzoic acid (4E1RCat), 5-[5-[(E)-(3-Benzyl-4-oxo-2-sulfanylidene-1,3-thiazolidin-5-ylidene)methyl]furan-2-yl]-2-chlorobenzoic acid (4E2RCat), α-[2-[4-(3,4-dichloro phenyl)-2-thiazolyl]-hydrazinylidene]-2-nitrobenzene-propanoic acid (4EGI-1), silvesterol, hippuristanol, rocaglates, pateamine A, elatol, sanguinarine, elisabatin A, 15d-PGJ2, eFT226, their analogs, and combinations thereof. In some embodiments, the protein synthesis inhibitor inhibits the initiation of translation elongation. In some embodiments, the agent effective in inhibiting the initiation of mRNA translation elongation comprises an agent effective in inhibiting or suppressing translation elongation, and/or the activity or expression of eEF2.

In yet another aspect, provided herein is a method of treating a urothelial cancer deficient in ARID-1A in a subject in need thereof. In some embodiments, the method comprises administering a therapeutically effective amount of an agent effective in suppressing or inhibiting mRNA translation elongation to the subject. In some embodiments, the agent effective in suppressing or inhibiting mRNA translation elongation comprises an agent effective in suppressing or inhibiting eukaryotic elongation factor 2 (eEF2).

In various embodiments, of the methods disclosed herein, an agent effective in suppressing or inhibiting eEF2 comprises any agent capable of slowing, interfering, inhibiting, reducing, preventing, or suppressing the activity or expression of eEF2. Preferably, the agent effective in preventing, suppressing or inhibiting eEF2 comprises an agent effective in suppressing or inhibiting eEF2 activity. In some embodiments, the agent effective in suppressing or inhibiting eEF2 activity comprises an agent capable of activating eEF2 kinase (eEF2K). In some embodiments, the agent capable of activating eEF2K decreases phosphorylation of eEF2K at serine 366 residue or increases phosphorylation of eEF2K at S398 of the eEF2K protein.

In some embodiments, the agent effective in suppressing or inhibiting eEF2 activity comprises an agent effective in suppressing or inhibiting the MAPK pathway. In some embodiments, the agent effective in suppressing or inhibiting the MAPK pathway comprises an agent effective in suppressing or inhibiting RASGRP1. In various embodiments of the methods disclosed herein, the agent effective in suppressing or inhibiting RASGRP1 comprises any agent capable of slowing, interfering, inhibiting, reducing, or suppressing the activity or expression of RASGRP1. Preferably, the agent effective in suppressing or inhibiting RASGRP1 comprises an agent effective in suppressing or inhibiting RASGRP1 transcript and/or protein.

In some embodiments, the method further comprises administering to the subject a therapeutically effective amount of a chemotherapeutic agent.

In various embodiments of the methods disclosed herein, the agent and the chemotherapeutic agents are individually a small molecule, a protein, a fusion protein, a peptide, a nucleic acid, an aptamer, an avimer, or derivatives or fragments thereof. In some embodiments, the agent is a peptide or a peptide mimetic. In some embodiments, two or more agents that suppress or inhibit mRNA translation elongation can be used in the methods disclosed herein. In some embodiments, the agent is a protein synthesis inhibitor.

In various embodiments of the above method the agent and the chemotherapeutic agent are administered concurrently.

According to yet another aspect, provided herein is a method of preventing uncontrolled cell growth and cancer progression of an ARID1A-deficient and/or ARID1A mutant tumor or cancer comprising contacting the tumor or cancer with an agent effective in increasing phosphorylation of eEF2 or decreasing eEF2 levels. In some embodiments, the ARID1A-deficient tumor or cancer is a urothelial cancer.

In yet another embodiment, provided herein is a method for inducing a transcription-translation conflict in an ARID1A mutant and/or ARID1A-deficient tumor cell of a subject, the method comprising contacting the ARID1A-deficient and/or ARID1A mutant tumor cell of the subject with an agent effective in inhibiting or suppressing protein synthesis. In some embodiments, the agent effective in inhibiting or suppressing protein synthesis comprises an agent effective in suppressing or inhibiting mRNA translation elongation. In some embodiments the agent comprises an agent effective in inhibiting or suppressing eEF2 expression or activity. In some embodiments, the agent effective in suppressing or inhibiting eEF2 activity is selected from agents effective in suppressing or inhibiting eEF2 activity, activating eEF2K, suppressing or inhibiting MAPK pathway, or inhibiting RASGRP1 expression.

In some embodiments, the agent comprises a protein synthesis inhibitor. In some embodiments, the protein synthesis inhibitor is selected from inhibitors targeting eEF1A, eEF1B, EFsec, eEF2, eIF5A, and eEF3. In an exemplary embodiment, the protein synthesis inhibitor inhibits eEF1A. Exemplary inhibitors include but are not limited to SR-A3 and Ternatin-4. In some embodiments, the protein synthesis inhibitor comprises agents that inhibit eIF4E, eIF4A, eIF4G, eIF2 complex function, or eIF3 complex function.

In some embodiments disclosed herein, ARID1A-deficiency comprises a low expression of ARID1A in a tumor or cancer cell as compared to the expression of ARID1A in a healthy or a non-cancerous cell.

In various embodiments disclosed herein, the subject is a mammal. In some embodiments, the mammal is a human.

These and other aspects of the present disclosure will be apparent to those of ordinary skill in the art in the following description, claims, and drawings.

### DESCRIPTION OF THE DRAWINGS

The patent or application file contains at least one drawing executed in color. Copes of this patent or patent application publication with color drawing(s) will be provided by the Office upon request and payment of the necessary fee.

The foregoing aspects and many of the attendant advantages of the embodiments described herein will become more readily appreciated as the same become better understood by reference to the following detailed description, when taken in conjunction with the accompanying drawings.

FIGS. 1A-1I illustrate loss of ARID1A leads to gene-specific transcriptional-translational conflict. Schema of UBC-Cre^{ERT} and K5-Cre^{ERT} Arid1a^{fl/fl} models is depicted in FIG. 1A. FIG. 1B shows pathway enrichment analysis of 262 mRNAs upregulated in the context of ARID1A loss. FIG. 1C is a heat map showing a subset of pro-proliferation upregulated mRNAs from Arid1a^{fl/fl} mice (>1.2 Log₂ fold change, FDR <0.05). FIG. 1D depicts hematoxylin and eosin (H&E) staining (left with keratin 5 inset) and quantification (right) of mouse bladder urothelial thickness (total (n=4/genotype) and basal cells (n=3/genotype)) in 400-day old WT and Arid1a^{fl/fl} mice. t-test. FIG. 1E shows immunohistochemistry (IHC) of ARID1A (left) and parallel sectioning and immunofluorescence (IF) of puromycin incorporation (right) in WT and Arid1a^{fl/fl} urothelium (DAPI=nuclei). n >6/genotype, >8,900 cells/genotype, t-test. FIG. 1F is a puromycin immunoblot of WT and Arid1a^{fl/fl} urothelial organoids (replicate of 3). FIG. 1G shows a schema of the polysome profiling assay (top). Polysome tracing showing an increase in polysome accumulation (left) and quantification showing an increase in the polysome (P) to sub-polysome (S) ratio (mean+/−S.E.M.). n=2/genotype, t-test. FIG. 1H is a waterfall plot showing polysome (P) to sub-polysome (S) ratio of 262 upregulated oncogenic mRNAs in Arid1a^{fl/fl} mice. FIG. 1I is a volcano plot of TMT mass spectrometry showing that 70% of upregulated mRNAs (105 out of 150—only 150 of the 262 genes were detected by TMT mass spectrometry) identified by RNA-seq (FIG. 1C) do not increase in protein abundance (<0.67 log₂ fold change and/or FDR >0.05, green dots) in Arid1a^{fl/fl} mice. Vertical lines demarcate log₂ fold change +/−0.67 and horizontal line demarcate FDR <or >0.05. All scale bars=100 μm.

FIGS. 2A-2H illustrate that ARID1A is a positive regulator of mRNA translation elongation. FIG. 2A shows immunoblots of regulators of mRNA translation in WT and Arid1a^{fl/fl} urothelial organoids (replicate of 3). FIG. 2B shows IHC and quantification of phospho-eEF2 (T56) and total eEF2, levels in WT and Arid1a^{fl/fl} bladder urothelium. n ≥3/genotype, t-test. FIG. 2C shows IHC or IF, and quantification of total eEF2K and phospho-eEF2K (S366) levels in WT and Arid1a^{fl/fl} bladder urothelium. Violin plot represents >9500 cells per genotype. n ≥4/genotype, t-test. FIGS. 2D-2E show IHC analysis of phospho-eEF2 (T56) and IF analysis of puromycin incorporation after treatment with A-484954 in Arid1a^{fl/fl} mice. Violin plot represents >7700 cells/genotype. n ≥4/treatment arm, t-test. FIGS. 2F-2G show IHC analysis of phospho-eEF2 (T56) and IF analysis of puromycin incorporation in Arid1a^{fl/fl} and Arid1a^{fl/fl};Eef2k^{-/-} mice. Violin plot represents >6000 cells/genotype. n ≥5/genotype, t-test. FIG. 2H shows Ribosome half-transit time in Arid1a^{fl/fl} and Arid1a^{fl/fl};Eef2k^{-/-} organoids (linear regression, left panels). Bar graph represents the average ribosome half-transit time of 3 independent experiments. PMS=post mitochondrial supernatants (complete+nascent proteins). PRS=post ribosomal supernatant (complete proteins), t-test. n.s.=not significant, scale bar=100 μm, mean±SEM.

FIGS. 3A-3K illustrate ARID1A regulates mRNA translation elongation through RASGRP1. FIG. 3A is a schematic of upstream signaling pathways that activate (AMPK pathway) or deactivate (PI3K and MAPK pathways) eEF2K. P-S366 and P-T56 are inhibitors. FIG. 3B shows IHC and quantification of phospho-MEK1/2, phosphor-ERK1/2, and phospho-p90RSK in WT and Arid1a^{fl/fl} urothelium. n ≥4/genotype, t-test. FIG. 3C shows RNA-seq mRNA expression of upstream MAPK regulators in WT (left bar) and Arid1a^{fl/fl} (right bar) urothelial cells. n=3/genotype, t-test. FIG. 3D shows RASGRP1 IHC and quantification in WT and Arid1a^{fl/fl} mice. n ≥5/genotype, t-test. FIG. 3E shows ARID1A ChIP QPCR of the Rasgrp1 promoter in WT and Arid1a^{fl/fl} organoids. n=2/genotype, t-test. FIGS. 3F-3G show IHC of RASGRP1 (right) and phospho-eEF2 (T56) (left) in WT and Rasgrp1^{-/-} mice. n ≥3/genotype, t-test. FIG. 3H show H3K27me3 CUT&Tag from WT (blue) and Arid1a^{fl/fl} (red) organoids. Black arrows=Rasgrp1 promoter. FIG. 3I shows QPCR of Rasgrp1 in Arid1a^{fl/fl} organoids after treatment with GSK126 (12.5 μM). t-test. FIG. 3J shows RASGRP1 IF in Arid1a^{fl/fl} organoids after treatment with GSK126. n=4/genotype, >50,000 cells/genotype, t-test. FIG. 3K shows Immunoblot analysis of phospho-eEF2 and total eEF2 in Arid1a^{fl/fl} organoids +/− GSK126. Each blot is representative of three biological replicates. n.s.=not significant, scale bar=100 μm, mean±SEM.

FIGS. 4A-4M illustrate Transcriptional-translational conflict is a tumor suppressive barrier. FIG. 4A shows a FACS analysis of ARID1A recombined cells (YFP⁺) in Arid1a^{fl/fl}, and Arid1a^{fl/fl}; Eef2k^{-/-} urothelial organoids over 9 successive passages (P2-P9). Representative ARID1A immunoblot from Arid1a^{fl/fl} and Arid1a^{fl/fl}; Eef2k^{-/-} urothelial organoids after two passages (P2) and 9 passages (P9). FIG. 4B shows H&E (left) and urothelial thickness (right) in WT, Eef2k^{-/-}, Arid1a^{fl/fl}, and Arid1a^{fl/fl}; Eef2k^{-/-} mice 400 days after tamoxifen. n ≥4/genotype, t-test. FIG. 4C shows clonogenic assay of Arid1a^{fl/fl}; Eef2k^{-/-} urothelial cells treated with an anti-FGFR3 antibody or the ODC1 inhibitor DFMO. n=8 biological replicates, t-test. FIG. 4D shows Kaplan-Meier survival curve of WT and Arid1a^{fl/fl} mice treated with BBN followed by tamoxifen. WT=13 mice, Arid1a^{fl/fl}=16 mice, Logrank test. FIG. 4E shows IF and quantification of puromycin incorporation in WT and Arid1a^{fl/fl} mice treated with BBN followed by tamoxifen (FIG. 4D). n=4/genotype, >30,000 cells/genotype. FIG. 4F shows Ki67 staining and quantification in WT and Arid1a^{fl/fl} tumors (FIG. 4D). n=4/genotype, t-test. FIG. 4G shows immunoblots of AURKB, KIF22, ODC1 and SKA1 in WT and Arid1a^{fl/fl} tumors (replicate of 3). FIG. 4H shows RNA-seq analysis of normal and cancer urothelial cells in WT and Ard1a^{fl/fl} backgrounds. Up or down regulated mRNAs are unique to ARID1A loss (DEGs=differentially expressed genes). FIG. 4I shows IHC and quantification of phospho-eEF2 (T56) in WT and Arid1a^{fl/fl} tumors. n ≥5/genotype, t-test. FIG. 4J shows representative images and quantification of human muscle invasive bladder cancer (MIBC) from the University of Washington showing high and low ARID1A protein levels (left panel) and corresponding phospho-eEF2 (T56). n(ARID1A high)=26; n(ARID1A low)=15, t-test. FIG. 4K shows representative images and quantification of MIBC obtained from the University of British Columbia showing high and low ARID1A protein levels (left panel) and corresponding phospho-eEF2 (T56). n(ARID1A high)=17; n(ARID1A low)=16, t-test. FIGS. 4L-4M show clinical staging and post-neoadjuvant chemotherapy pathologic staging of FIGS. 4J-K show patients separated by ARID1A and p-eEF2 levels (n(low)=31, n(high)=43). Chi-square test. n.s.=not significant, scale bar=100 μm, mean±SEM.

FIGS. 5A-5I illustrate pharmacologic inhibition of translation elongation initiation inhibits growth of ARID1A-deficient, but not proficient tumors. FIG. 5A shows WT and Arid1a^{fl/fl} tumor organoids cell viability after treatment with HHT (CellTiter-Glo® 2.0). n ≥3/genotype, t-test. FIG. 5B shows cell viability of ARID1A proficient and deficient human bladder cancer cell lines treated with HHT. n=3/genotype, t-test. FIG. 5C shows schematic of patient derived xenograft (PDX) model generation. Representative ARID1A IHC from PDX1 (ARID1A low), PDX2 (ARID1A medium) and PDX3 (ARID1A high) tumor tissues. FIGS. 5D-5F show tumor growth rate in PDX1 (ARID1A low), PDX2 (ARID1A medium), and PDX3 (ARID1A high) models treated with HHT (0.7 mg/kg; twice/day). n ≥10/treatment arm. FIG. 5G shows HHT or vehicle (PBS) PDX1 (ARID1A low) Kaplan-Meier survival curve. n=10/arm, Logrank test. FIG. 5H shows percent CC3 positive cells from the PDX1 (ARID1A low) group treated with HHT or vehicle (PBS). n=6/arm, t-test. FIG. 5I shows percent Ki67 positive cells from the PDX1 (ARID1A low) group treated with HHT or vehicle (PBS). n=6/treatment arm, t-test. n.s.=not significant, mean±SEM.

FIGS. 6A-6K illustrate ARID1A loss leads to a decrease in the translation of DNA damage response mRNAs. FIG. 6A shows a volcano plot showing 278 translationally stalled mRNAs (orange dots, increased P/S ratio), which include Brca2, Ercc1, Ercc2, and Fancc (FDR <0.05). FIGS. 6B-6C show protein (replicate of 2) and mRNA (replicate of 3) levels of DNA damage response genes in WT and Arid1a^{fl/fl} urothelial cells. In FIG. 6C genes in WT urogenital cells is presented by the left bar and genes in Arid1a^{fl/fl} urogenital cells is presented by the right bar. FIG. 6D shows protein levels of DNA damage response genes in Arid1a^{fl/fl} and Arid1a^{fl/fl};Eef2k^{-/-} urothelial cells (replicate of 3). FIGS. 6E-6F shows γH2AX or CC3 staining and quantification in WT and Arid1a^{fl/fl} mice after tamoxifen administration followed by 9 days of BBN treatment. Urothelium is marked with red dotted lines. n ≥4/genotype, t-test. FIG. 6G shows representative comet assay showing increased DNA damage (tail length) in Arid1a^{fl/fl} organoids compared to WT organoids treated with BCPN. FIG. 6H shows Mass spectrometry measurements of urine BCPN in WT and Arid1a^{fl/fl} mice after 9 days of BBN treatment (n ≥3/genotype). FIG. 6I shows pie chart showing tumor outcome in WT and Arid1a^{fl/fl} mice treated with BBN for 150 days after ARID1A deletion (WT=11 and Arid1a^{fl/fl}=12). FIG. 6J shows H&E of tumors from WT and Arid1a^{fl/fl} mice after 150 days of BBN treatment. Tumor area is marked with yellow dotted lines. WT=6 tumors and Arid1a^{fl/fl}=5 tumors, t-test. FIG. 6K shows ARID1A IHC of Arid1a^{fl/fl} tumors after tamoxifen followed by 150 days of BBN treatment (FIG. 6I). Tumor area is marked with yellow dotted lines. n.s.=not significant, scale bar=100 μm, mean±SEM.

FIGS. 7A-7H illustrate restoration of translation elongation is necessary to enable carcinogenesis in ARID1A-deficient urothelium. FIGS. 7A-7B show γH2AX and CC3 staining and quantification in Arid1a^{fl/fl} and Arid1a^{fl/fl};Eef2k^{-/-} mouse urothelium (outlined in red) after tamoxifen administration followed by 9 days of BBN treatment. n ≥6/genotype, t-test. FIGS. 7C-7D show γH2AX and CC3 staining and quantification in Arid1a^{fl/fl} mouse urothelium (outlined in red) after tamoxifen administration and pre-treatment with A-484954 followed by a 9-day BBN treatment. n ≥4/arm, t-test. FIG. 7E shows schematic of tamoxifen treatment followed by 150 days of BBN treatment in Arid1a^{fl/fl} and Arid1a^{fl/fl};Eef2k^{-/-} mice. FIG. 7F shows tumor size after tamoxifen followed by 150 days of BBN treatment in Arid1a^{fl/fl} and Arid1a^{fl/fl};Eef2k^{-/-} mice. n ≥6/genotype, t-test. FIG. 7G shows percent ARID1A (+) cells in Arid1a^{fl/fl} and Arid1a^{fl/fl};Eef2k^{-/-} mice after tamoxifen followed by 150 days of BBN treatment. n ≥6/genotype, t-test. FIG. 7H shows ARID1A IHC of Arid1a^{fl/fl};Eef2k^{-/-} tumors after tamoxifen followed by 150 days of BBN treatment. This demonstrates the presence of ARID1A-null tumors compared to FIG. 6K. Scale bar=100 μm, mean±SEM.

FIG. 8 shows simplified schematic of the mechanistic underpinning of transcriptional-translational conflict and a potential mechanism of gene expression parity.

### DETAILED DESCRIPTION

SWI/SNF (SWItch/Sucrose Non-Fermentable), is a nucleosome remodeling complex found in eukaryotes that associate to remodel the way DNA is packaged. It is composed of several proteins products of the SWI and SNF genes (SWI1, SWI2/SNF2, SWI3, SWI5, SWI6) as well as other polypeptides. It possesses a DNA-stimulated ATPase activity and can destabilize histone-DNA interactions in reconstituted nucleosomes in an ATP-dependent manner. The human analogs of SWI/SNF are BAF (SWI/SNF-A) and PBAF (SWI/SNF-B). Several studies revealed that subunits of the SWI/SNF mammalian complex are frequently mutated in human cancers. Of these, the AT-rich interactive domain-containing protein 1A (ARID1A), subunit of the SWI/SNF complex is the most mutated in human cancers. ARID1A is a member of the SWI/SNF family, whose members have helicase and ATPase activities and are thought to regulate transcription of certain genes by altering the chromatin structure around those genes.

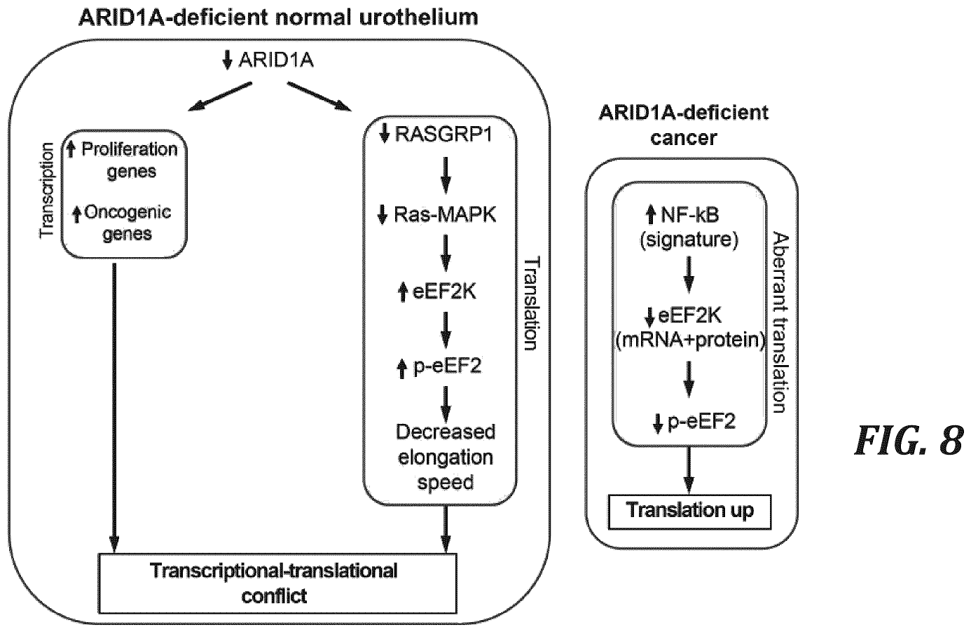

The inventors have discovered a tumor suppressive process by which loss of ARID1A triggers an increase in a nexus of pro-proliferation transcripts, but a simultaneous inhibition of the eukaryotic elongation factor (eEF2), which results in tumor suppression. The resulting transcriptional-translational conflict specifically restrains the synthesis of pro-proliferation and mitogenic proteins and collateral DNA damage response genes thereby preventing uncontrolled cell growth and cancer progression. The inventors demonstrate that ARID1A functions as a positive regulator of translation elongation. This is a surprising and an unexpected finding because major tumor suppressors have been shown to negatively, but not positively regulate the protein-synthetic capacity of cells. For example, TP53 binds to and inhibits the rRNA methyl-transferase fibrillarin which prevents IRES-mediated translation of cancer genes. Loss of PTEN within prostate epithelium promotes cancer progression through hyperactivation of the mTOR-eIF4F signaling pathway and translation of pro-metastasis mRNAs. APC loss leads to intestinal tumorigenesis driven by enhanced translation elongation of cyclin D mRNA. Contrary to these tumor suppressors, the inventors show herein that ARID1A is necessary for the maintenance of translation elongation through eEF2 (FIG. 8). Therefore, loss of ARID1A has significant negative implications for the ability of upregulated proliferation and oncogenic mRNA networks to be synthesized into proteins. Further, it was discovered that resolution of this transcriptional-translational conflict through enhancing translation elongation speed enables the efficient and precise synthesis of a network of poised mRNAs resulting in uncontrolled proliferation, clonogenic growth, and cancer progression. The inventors observed similar phenomenon in patients with ARID1A low tumors, which also exhibit increased translation elongation activity through eEF2. These discoveries reveal an oncogenic stress created by transcriptional-translational conflict which forms a functional barrier to uncontrolled cell growth and cancer progression and provide a unified gene expression model that unveils the importance of the crosstalk between transcription and translation in promoting cancer.

This concept has important parallels with other major oncogenic signaling pathways. For example, it has been shown that loss of the tumor suppressor PTEN can upregulate TP53 levels which promotes cellular senescence and prevents the formation of lethal prostate cancer. However, combined loss of PTEN and TP53 can overcome this barrier leading to uninhibited tumor growth. This is also true for oncogenes such as MYC and PIK3CA. MYC, which is commonly amplified in human malignancies, causes apoptosis in the context of overexpression in normal cells. This is mediated through enhanced expression of the apoptosis regulator BIM. Importantly, co-expression of MYC and the anti-apoptotic and inhibitor of BIM, BCL-2, leads to a significant acceleration in tumorigenesis. Activated PIK3CA causes cellular differentiation in epidermal progenitors which is mediated by the AKT substrate SH3RF1. This leads to terminal differentiation of epidermal cells that possess activating mutations of PIK3CA and prevents oncogenic clonal expansion. In line with these oncogenic stress pathways, the present disclosure shows that ARID1A loss leads to a significant reduction in eEF2 activity which promotes a conflict between the expression of oncogenic genes and their subsequent translation (FIG. 8). Restoration of translation elongation enabling gene expression parity is sufficient to unleash the oncogenic properties of ARID1A deficiency leading to uncontrolled cell growth and tumor progression.

The relationship between ARID1A and protein synthesis may be a common theme in enabling transformation or cancer progression in the context of ARID1A loss. ARID1A deficiency by itself does not drive transformation in murine ovary, liver, lung, or pancreas, and human gastric organoids. However, it has been shown in these tissues that co-deletion of the tumor suppressor PTEN, overexpression of the oncogene MYC, or expression of oncogenic KRAS or PIK3CA along with ARID1A loss can drive tumorigenesis and cancer progression. All these oncogenic signaling pathways that synergize with ARID1A loss are regulators of protein synthesis. Therefore, gaining the ability to synthesize proteins efficiently is a key parameter for enabling the oncogenic properties of ARID1A loss. The inventors demonstrate that ARID1A deficiency leads to a selective decrease in the translation of key oncogenic factors and DNA damage repair machinery. Loss of ARID1A also leads to a decrease in de novo protein synthesis within human prostate epithelial cells and murine bone marrow derived stroma.

Moreover, the inventors identify a therapeutic vulnerability unique to ARID1A-deficient and/or ARID1A mutant tumors. Given that rescue of translation elongation is needed to drive ARID1A-deficient tumor progression, it was rationalized that this may provide a therapeutic window using an FDA approved translation inhibitor. Using three model systems, the inventors demonstrate herein, that ARID1A-deficient but not proficient cancers are exquisitely sensitive to translation inhibition. Remarkably, ARID1A-null tumors retain a memory of this translational dependence, which represents a selective therapeutic vulnerability. It has also been shown that SWI/SNF defective cancer cell lines and rhabdoid tumors are also sensitive to PI3K pathway inhibitors or HHT. These findings demonstrate how ARID1A mutation and/or ARID1A deficiency or SWI/SNF complex mutations/defects create a synthetic lethal relationship with mRNA translation which can be targeted to inhibit tumor growth.

Accordingly, in one aspect, provided herein is a method for treating SWI/SNF chromatin remodeling mutant tumors or cancers in a subject in need thereof. The method comprises administering to the subject a therapeutically effective amount of at least one agent effective in suppressing or inhibiting mRNA translation. Any agent capable of slowing, interfering, inhibiting, reducing, or suppressing mRNA translation is contemplated by the methods disclosed herein. In an embodiment, the tumors or cancers comprise ARID1A-deficient and/or ARID1A mutant tumors or cancers. The method comprises administering to the subject a therapeutically effective amount of an agent effective in suppressing or inhibiting mRNA translation elongation. In some embodiments, the agent effective in suppressing or inhibiting mRNA translation elongation comprises an agent effective in suppressing or inhibiting eukaryotic elongation factor 2 (eEF2). In some embodiments, the method further comprises determining the presence of SWI/SNF chromatin remodeling complex mutation or ARID-1a deficiency in the tumor or cancers cells of the subject prior to administering the agent effective in suppressing or inhibiting mRNA translation elongation. In various embodiments of the above method, the step of determining the presence of the SWI/SNF chromatin remodeling complex mutation or the ARID1A deficiency in the tumor or cancer cells of the subject and the step of administering the agent to the subject occur simultaneously or concurrently.

In another aspect, provided herein is a method of selecting and/or deselecting a subject having a tumor or cancer for treatment with a therapeutic agent, the method comprising: (i) obtaining a biological specimen from the tumor or cancer of the subject; (ii) determining the presence or absence of a SWI/SNF chromatin remodeling complex mutation or an ARID1A deficiency in the biological specimen; and (iii) administering an effective amount of the therapeutic agent to the subject determined to have the SWI/SNF chromatin remodeling complex mutation and/or the ARID1A-deficiency. In some embodiments, the ARID1A deficiency comprises a low expression of ARID1A in the biological sample obtained from the tumor or cancer of the subject as compared to the expression of ARID1A in a corresponding biological sample obtained from a healthy subject. The biological sample may be a fluid sample, such as blood, serum plasma, ascites, sputum, saliva, or urine. The biological sample may be a tissue sample, such as a cancer, pre-cancer or benign tumor tissue sample.

According to yet another aspect, provided herein is a method of preventing uncontrolled cell growth and cancer progression of an ARID1A-deficient and/or ARID1A mutant tumor or cancer. In some embodiments, the method comprises contacting the ARID1A-deficient and/or ARID1A mutant tumor or cancer with an agent effective in suppressing or inhibiting the activity or expression of eEF2. In some embodiments, the method comprises contacting the ARID1A-deficient and/or ARID1A mutant tumor or cancer with an agent effective in increasing phosphorylation of eEF2.

As discussed herein, any agent capable of slowing, interfering, inhibiting, reducing, preventing, or suppressing mRNA translation elongation can be used in the methods disclosed herein. Preferably, the agent capable of slowing, interfering, inhibiting, reducing, preventing, or suppressing mRNA translation elongation is an agent capable of suppressing or inhibiting this process. In some embodiments, the agent effective in suppressing or inhibiting mRNA translation elongation comprises an agent effective in preventing, suppressing, or inhibiting the activity of eEF2. In some embodiments, the agent effective in suppressing or inhibiting the activity of eEF2 is an agent effective in activating eEF2 kinase (eEF2K). Any agent or substance that stimulates or increases the activity of eEF2K can be used in the methods disclosed herein. In some embodiments, the agent effective in activating eEF2K decreases phosphorylation of eEF2K at serine residue at position 366 (S366) and/or increase phosphorylation at the serine residue at position 398 (S398) of the eEF2K protein. An agent effective in activating eEF2K also includes any agent effective in suppressing or inhibiting p387/6 MAPK.

In some embodiments, the agent comprises an agent effective in suppressing or inhibiting the MAPK pathway. In some embodiments, the agent comprises an agent effective in suppressing or inhibiting RASGRP1. An agent effective in suppressing or inhibiting RASGRP1 comprises any agent capable of slowing, interfering, inhibiting, reducing, or suppressing the activity or expression of RASGRP1. Preferably, an agent effective in suppressing or inhibiting RASGRP1 comprises an agent effective in suppressing or inhibiting RASGRP1 transcript and/or protein. Any agent effective in activating eEF2K activity and/or suppressing or inhibiting p387/6 MAPK pathway can be used in the methods disclosed herein. Suitable agents that can be used in the methods disclosed herein, include agents that disrupt the MAPK pathway, target RASGRP1, activate eEF2 kinase or increase eEF2 phosphorylation.

In some embodiments, the methods disclosed herein further comprise administering to the subject a therapeutically effective amount of at least one additional cancer therapy. In some embodiments, the at least one additional therapy is selected from radiotherapy, immunotherapy, surgery, hormonal therapy, toxin therapy or chemotherapy. In some embodiments, the at least one additional therapy comprises a chemotherapeutic agent. In some embodiments, the at least one additional therapy comprises radiotherapy.

In various embodiments of the methods disclosed herein, the agents effective in suppressing or inhibiting mRNA translation, mRNA translation elongation, suppressing or inhibiting eEF2, activating eEF2K, suppressing or inhibiting RASGRP1, and the chemotherapeutic agents are individually a small molecule, a protein, a fusion protein, a peptide, a nucleic acid, an aptamer, an avimer, or a derivative or fragment thereof. In some embodiments, the agent is a peptide or a peptide mimetic.

In various embodiments, the agents or therapeutic agents disclosed herein comprise a protein synthesis inhibitor. In some embodiments, the agent or therapeutic agent is capable of inhibiting or suppressing mRNA translation. In some embodiments, the protein synthesis inhibitor is selected from inhibitors targeting eEF1A, eEF1B, EFsec, eEF2, eIF5A, eEF3 eIF4E, eIF4A, eIF4G, eIF2 complex function, or eIF3 complex function. In some embodiments, the agent or therapeutic agent is selected from an agent that is a cap dependent translation inhibitor, a ribosome translocation inhibitor, a ribosome targeting peptide, a peptide bond inhibitor, an inhibitor of aa-tRNA delivery, and an agent capable of locking tRNA-ribosome complex.

Exemplary compounds include but are not limited to homoharringtonine (omacetaxine mepesuccinate), eFT508, eFT226, 4-[(3E)-3-[[5-(4-nitrophenyl)furan-2-yl]methylidene]-2-oxo-5-phenylpyrrol-1-yl]benzoic acid (4E1RCat), 5-[5-[(E)-(3-Benzyl-4-oxo-2-sulfanylidene-1,3-thiazolidin-5-ylidene)methyl]furan-2-yl]-2-chlorobenzoic acid (4E2RCat), α-[2-[4-(3,4-Dichlorophenyl)-2-thiazolyl]-hydrazinylidene]-2-nitrobenzene-propanoic acid (4EGI-1), silvesterol, hippuristanol, rocaglates, pateamine A, elatol, sanguinarine, elisabatin A, 15d-PGJ2, eFT226, their analogs, and combinations thereof.

In various embodiments of the methods disclosed herein, the agent and the chemotherapeutic agent are co-administered.

In various embodiments of the methods disclosed herein, the tumor or cancer is selected from a cancer of the brain, breast, bladder, bone, cartilage, cervix, colon, neural tissue, glia, esophagus, fallopian tube, pancreas, intestines, gallbladder, kidney, liver, lung, ovary, pancreas, parathyroid, pineal gland, pituitary gland, prostate, spinal cord, spleen, skeletal muscle, skin, muscle, stomach, testis, thymus, thyroid, urogenital tract, ureter, urethra, uterus, endometrium, vagina, or combination thereof. In some embodiments, the tumor or cancer is a urothelial cancer.

SWI/SNF mutant tumors or cancers include tumors or cancers exhibiting a decreased function of SWI/SNF due to genomic mutations or down regulation of component proteins at the transcript and/or protein level. SWI/SNF mutations include but are not limited to inactivation of at least one SWI/SNF component or loss of at least one SWI/SNF component due to epigenetic regulation, transcriptional regulation and/or post-translational modifications (PTMs).

The terms ARID1A-deficient and/or ARID1A mutant tumors or cancers are used interchangeably to include ARID1A-deficient or -low tumors exhibiting ARID1A-based mutations or down regulation of ARID1A transcript or protein. An ARID1A-deficiency in a tumor or cancer includes but is not limited to inactivation of ARID1A or loss of ARID1A expression due to epigenetic regulation, transcriptional regulation and/or post-translational modifications (PTMs). In some embodiments, ARID1A-deficiency comprises a lower expression level of ARID1A in a tumor or cancer cell. Methods for determining expression levels of ARID1A are known in the art and include but are not limited to determining expression level at a protein, transcript, and/or genome level. Such methods include but are not limited to mass spectrometry, immunoblotting, immunofluorescence or immunohistochemistry, RNA in situ hybridization, Quantitative PCR, exome sequencing, whole genome sequencing (WGS), targeted exome sequencing, chromatin-based methods such as assay for transposase-accessible chromatin with sequencing (ATAC-seq), and promoter-based studies focusing on histone methylation status.

In yet another aspect, provided herein is a method of treating an ARID1A-deficient and/or ARID1A mutant urothelial cancer in a subject in need thereof. In some embodiments, the method comprises administering to the subject a therapeutically effective amount of an agent effective in suppressing or inhibiting mRNA translation elongation. In some embodiments, the ARID1A deficiency comprises a low or no expression of ARID1A in a biological sample obtained from the subject with urothelial cancer. In some embodiments, the agent effective in preventing, suppressing or inhibiting mRNA translation elongation comprises an agent effective in suppressing or inhibiting the activity or expression of the eukaryotic elongation factor 2 (eEF2). Preferably, the agent effective in suppressing or inhibiting mRNA translation elongation comprises an agent effective in suppressing or inhibiting the activity of eEF2. In some embodiments, the methods disclosed herein further comprise administering to the subject a therapeutically effective amount of at least one additional cancer therapy.

In yet another aspect, provided herein is a method for inducing a transcription-translation conflict in an ARID1A-deficient and/or ARID1A mutant tumor cell of a subject. In some embodiments, the method comprises contacting the tumor cell of the subject with an agent effective in preventing, inhibiting or suppressing mRNA translation elongation. In some embodiments, ARID1A-deficient and/or ARID1A mutant tumor cell of the subject comprises a low expression of ARID1A. In some embodiments, the agent effective in inhibiting or suppressing mRNA translation elongation comprises an agent effective in suppressing or inhibiting eEF2 activity or gene expression. Preferably, the agent effective in inhibiting or suppressing mRNA translation elongation comprises an agent capable of suppressing or inhibiting eEF2 activity.

In various embodiments, the agent capable of suppressing or inhibiting eEF2 activity is selected from agents effective in activating eEF2K activators, suppressing or inhibiting MAPK pathway, or inhibiting RASGRP1 expression. The agents that inhibit the activity of eEF2 can work by any known mechanism. Agents that inhibit eEF2 complex formation, eEF2 activity, or eEF2-dependent mRNA translation can be used in the methods disclosed herein. In certain embodiments of the methods disclosed herein, the agent that inhibits the activity of the eEF2 translation elongation factor is an agent that blocks binding of eEF2 to mRNA. In some embodiments, suitable agents include peptide mimetics of eEF2. In other embodiments, the agents include eEF2 inhibitors and mTOR inhibitors. In some embodiments, two or more agents that inhibit the activity of eEF2 can be used in the methods disclosed herein.

In some embodiments, the agents disclosed herein comprise small molecules, proteins, fusion proteins, peptides, nucleic acids, aptamers, avimers, or derivatives or fragments thereof. In some embodiments, the agents disclosed herein are capable of inhibiting or suppressing mRNA translation. In some embodiments, the agent is a protein synthesis inhibitor.

In some embodiments, the agent is a peptide or a peptide mimetic. In some embodiments, peptide mimetics suitable for use in the methods disclosed herein include stapled peptides. In some embodiments, the stapled peptide is a hydrocarbon-stapled peptide. As used herein, a “stapled peptide” is a peptide that comprises a synthetic brace (“staple”) moiety. Peptide stapling is used to enhance pharmacologic performance of peptides by locking the peptide in a specific conformation. Stapled peptides can be prepared according to the methods known in the art, for example, by incorporating olefin terminated, non-natural amino acids as building blocks into a peptide precursor and forming a stapled peptide by reacting one olefin groups with an adjacent olefin group, e.g., as described in Walensky L D, Bird G H. Hydrocarbon-stapled peptides: principles, practice, and progress. J Med Chem. 2014; 57(15):6275-6288, the disclosure of which is incorporated herein by reference.

Suitable agents suitable for use in the methods disclosed herein include but are not limited to homoharringtonine (omacetaxine mepesuccinate), eFT508, eFT226, 4-[(3E)-3-[[5-(4-nitrophenyl)furan-2-yl]methylidene]-2-oxo-5-phenylpyrrol-1-yl] benzoic acid (4E1RCat), 5-[5-[(E)-(3-Benzyl-4-oxo-2-sulfanylidene-1,3-thiazolidin-5-ylidene)methyl]furan-2-yl]-2-chlorobenzoic acid (4E2RCat), α-[2-[4-(3,4-Dichloro phenyl)-2-thiazolyl]-hydrazinylidene]-2-nitrobenzene-propanoic acid (4EGI-1), silvesterol, hippuristanol, rocaglates, pateamine A, elatol, sanguinarine, elisabatin A, 15d-PGJ2, eFT226, their analogs, and combinations thereof. In some embodiments, two or more agents that inhibit mRNA translation may be used. In some embodiments, each of the two or more agents target different stages of mRNA translation.

Agents used in the methods disclosed herein can be administered in any suitable manner known in the art. The agents used in the methods disclosed herein can be administered orally or parenterally and can be in the form of a solid preparation (tablets, capsules, granules, fine granules, powders, etc.) or a liquid preparation (syrups, injections, and the like) supplemented with pharmaceutically acceptable carriers. Various organic or inorganic carrier substances routinely used as pharmaceutical materials are used as the pharmaceutically acceptable carriers. Solid formulations typically comprise an excipient, a lubricant, a binder, a disintegrant, or mixtures thereof. Liquid formulations typically comprise a solvent, a solubilizer, a suspending agent, a tonicity agent, a pH adjuster, a buffering agent, a soothing agent, or combinations thereof. Pharmaceutical additives such as antiseptics, antioxidants, colorants, and/or sweeteners can be further included in the formulations. Examples of the excipients include lactose, saccharose, D-mannitol, starch, crystalline cellulose, and light anhydrous silicic acid. Examples of the lubricant include magnesium stearate, calcium stearate, talc, and colloidal silica. Examples of suitable binders include crystalline cellulose, saccharose, D-mannitol, dextrin, hydroxypropylcellulose, hydroxypropylmethylcellulose, and polyvinylpyrrolidone. Examples of suitable disintegrants include starch, carboxymethylcellulose, calcium carboxymethylcellulose, sodium croscarmellose, and sodium carboxymethyl starch. Examples of suitable solvents include injectable water, alcohols, propylene glycol, Macrogol, sesame oil, and corn oil. Examples of suitable solubilizers include polyethylene glycol, propylene glycol, D-mannitol, benzyl benzoate, ethanol, trisaminomethane, cholesterol, triethanolamine, sodium carbonate, and sodium citrate. Suitable suspending agents include surfactants, such as stearyl triethanolamine, sodium lauryl sulfate, lauryl aminopropionic acid, lecithin, benzalkonium chloride, benzethonium chloride, and glycerin monostearate, and hydrophilic polymers such as polyvinyl alcohol, polyvinylpyrrolidone, sodium carboxymethylcellulose, methylcellulose, hydroxymethylcellulose, hydroxyethylcellulose, and hydroxypropylcellulose. Suitable tonicity agents include sodium chloride, glycerin, and D-mannitol, and suitable buffering agents include phosphate, acetate, carbonate, and citrate buffer solutions.

Examples of dosage forms suitable for parenteral administration can include injections, drops, suppositories, percutaneous absorption formulations, transmucosal absorption formulations, and inhalants for intravenous administration, intracutaneous administration, subcutaneous administration, or intramuscular administration. Examples of dosage forms suitable for oral administration can include capsules, tablets, and syrups. When the therapeutic agent of the present methods is a peptide compound, its dosage form is preferably a dosage form suitable for parenteral administration, for example, an injection, drops, or an inhalant. Various such dosage forms are known to those skilled in the art. Those skilled in the art can appropriately select a dosage form suitable for the desired administration route and can produce a preparation in the form of a pharmaceutical composition using, if necessary, one or two or more pharmaceutical additives that may be used in the art. Alternatively, a peptide compound can be orally administered in the form of a preparation unsusceptible to digestion in the gastrointestinal tract, for example, in the form of a microcapsule comprising the active ingredient peptide enclosed in a liposome. Another possible administration method involves absorption through a mucosal membrane other than the gastrointestinal mucosa, such as rectal mucosa, nasal mucosa, or hypoglossal mucosa. In this case, the agent can be administered in the form of a suppository, a nasal spray, an inhalant, or a sublingual tablet to the individual. Alternatively, a preparation improved in terms of the retention of the peptide in blood by the adoption of, for example, a controlled-release preparation or a sustained release preparation containing a polysaccharide such as dextran or a biodegradable polymer typified by polyamine or PEG as a carrier can also be used in the methods disclosed herein.

When the methods include administration of more than one therapeutic agent or drug, the agents can be administered simultaneously or almost simultaneously (e.g., within 1 hour) or can be administered in a staggered manner at an interval of several hours. For example, a first drug is administered every day, immediately followed by the administration of a second drug. Typically, the first and second drugs are administered at times suitable for these drugs to exert their effects. In this way, these drugs can be used in combination.

Efficacy of the agents and combinations of therapeutic agents described herein in treating, preventing and/or managing the indicated diseases or disorders can be tested using various models known in the art, which provide guidance for treatment of human disease. For example, models for determining efficacy of treatments for ovarian cancer are described, e.g., in Mullany et al. Endocrinology 2012, 153:1585-1592; and Fong et al. J. Ovarian Res. 2009, 2:12. Models for determining efficacy of treatments for pancreatic cancer are described in Herreros-Villanueva et al. World J. Gastroenterol. 2012, 18:1286-1294. Models for determining efficacy of treatments for breast cancer are described, e.g., in Fantozzi, Breast Cancer Res. 2006, 8:212. Models for determining efficacy of treatments for melanoma are described, e.g., in Damsky et al. Pigment Cell Melanoma Res. 2010:23:853-859. Models for determining efficacy of treatments for lung cancer are described, e.g., in Meuwissen et al. Genes Devel., 2005, 19:643-664. Models for determining efficacy of treatments for lung cancer are described, e.g., in Kim, Clin. Exp. Otorhinolaryngol. 2009, 2:55-60; and Sano, Head Neck Oncol. 2009, 1, 32. Models for determining efficacy in B cell lymphomas, such as diffuse large B cell lymphoma (DLBCL), include the PiBCL1 murine model with BALB/c (haplotype H-2d) mice. Illidge et al. Cancer Biother. Radiopharm. 2000, 15:571-580. Efficacy of treatments for Non-Hodgkin's lymphoma may be assessed using the 38C13 murine model with C3H/HeN (haplotype 2-Hk) mice or alternatively the 38C13 Her2/neu model. Timmerman et al. Blood 2001, 97:1370-1377; Penichet et al. Cancer Immunolog. Immunother. 2000, 49:649-662. Efficacy of treatments for chronic lymphocytic leukemia (CLL) may be assessed using the BCL1 model using BALB/c (haplotype H-2d) mice. Dutt et al. Blood 2011, 117:3230-3239.

As used herein, the term “about” indicates that the subject value can be modified by plus or minus 5% and still fall within the disclosed embodiment.

The term “effective amount” or “therapeutically effective amount” refers to that amount of a compound or combination of compounds as described herein that is sufficient to affect the intended application including, but not limited to, disease or cancer treatment. A therapeutically effective amount may vary depending upon the intended application (in vitro or in vivo), or the human subject and disease condition being treated (e.g., the weight, age, and gender of the subject), the severity of the disease condition, the manner of administration, and the like which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the present disclosure are used to delay development of a disease or to slow the progression of a disease.

The terms “co-administration,” “co-administering,” “administered in combination with,” “administering in combination with,” “simultaneous,” and “concurrent,” as used herein, encompass administration of two or more active pharmaceutical ingredients to a human subject so that both active pharmaceutical ingredients and/or their metabolites are present in the human subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present is also encompassed in the methods disclosed herein.

The term “disease”, “cancer”, or “tumor” as used herein include both solid and hematologic cancers, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, a neoplasm of the central nervous system (CNS), a spinal axis tumor, a brain stem glioma, glioblastoma multiforme, an astrocytoma, a schwannoma, an ependymoma, a medulloblastoma, a meningioma, a squamous cell carcinoma, a pituitary adenoma and a Ewings sarcoma, including refractory versions of any of the above cancers, or a combination of one or more of the above cancers.

The term “biological sample” as used herein includes both fluid samples and tissue samples. Exemplary fluid samples include but are not limited to blood, ascites, serum, plasma, urine, sputum, and saliva. Tissue samples include a cancer or tumor tissue sample, such as a tumor biopsy, and a pre-cancer or benign tumor tissue sample.

The term “chemotherapy” refers to the use of a drug to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard: nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (preferably T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

The term “Radiotherapy” refers to the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiation therapy used according to the present disclosure may include, but is not limited to, the use of γ-rays, X-rays, and/or the directed delivery of radioisotopes to tumor cells. Radiotherapy may comprise the use of radiolabeled antibodies to deliver doses of radiation directly to the cancer site (radioimmunotherapy).

The term “immune therapy” or “immunotherapy” refers to the treatment of cancer or other diseases with an agent or agents that help your immune system prevent or slow cancer cell growth or fight an infection or other disease. The treatments and/or immunotherapy agent is typically a biological agent or therapy that uses substances made from living organisms, including humans. Currently, immunotherapy include immune checkpoint inhibitors; T cell transfer therapy, monoclonal antibodies, cytokines, immune modulators, and vaccines. Immune checkpoint inhibitors include, for example, pembrolizumab, nivolumab, cemiplimab, atezolizumab, avelumab, duralumab, lpilimumab, and relatlimab. Monoclonal antibodies against cancer specific or cancer associated antigens can also be used. Typically, the monoclonal antibodies are used with or without a conjugated drug or radionuclide. Examples of monoclonal antibodies include those specific for an immune checkpoint inhibitor, Her2, CD30, and the like. Immunotherapeutic cytokines can include interleukins or interferons such as IL-2, IFNα, IFNβ, or IFNγ. Cancer vaccines include, for example, sipuleucel-T and talimogene laherparepvec, dendritic cell vaccines, and the like.

The term “Hormone therapy” as used herein refers to the treatment of a cancer that uses a hormone to grow, and in particular, include breast and prostate cancers. The treatment typically comprises administering a hormone to slow or stop growth of a tumor. The hormones administered typically can comprise gonadotropin-releasing hormone agonists such as goserelin and leuprolide; aromatase inhibitors, such as anastrozole, letrozole, and exemestane; and selective estrogen receptor modulators, including, for example, tamoxifen and toremifene; and the like.

While each of the elements of the present disclosure is described herein as containing multiple embodiments, it should be understood that, unless indicated otherwise, each of the embodiments of a given element of the present disclosure is capable of being used with each of the embodiments of the other elements of the present disclosure and each such use is intended to form a distinct embodiment of the present disclosure.

The referenced patents, patent applications, and scientific literature referred to herein are hereby incorporated by reference in their entirety as if each individual publication, patent or patent application were specifically and individually indicated to be incorporated by reference.

While illustrative embodiments have been illustrated and described, it will be appreciated that various changes can be made therein without departing from the spirit and scope of the disclosure.

As can be appreciated from the disclosure above, the present embodiments have a wide variety of applications. The embodiments of the disclosure are further illustrated by the following examples, which are only illustrative and are not intended to limit the definition and scope of the embodiments disclosure herein in any way.

### Example 1

### Loss of ARID1A Leads to a Gene Specific Transcriptional-Translational Conflict in Urothelium

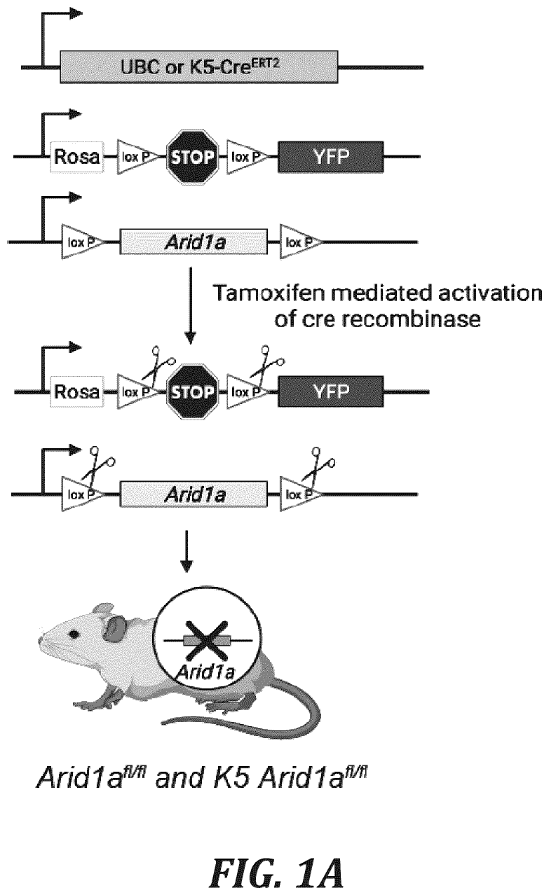

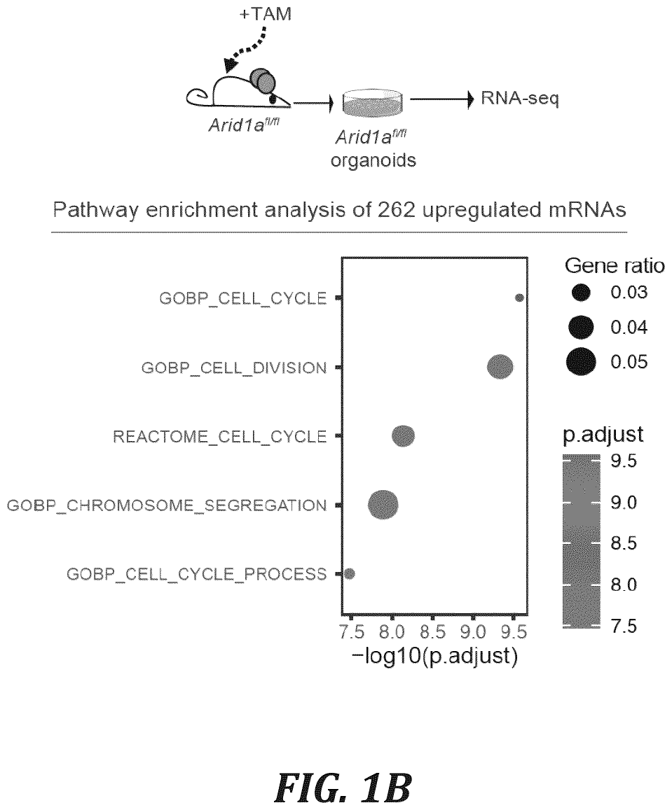

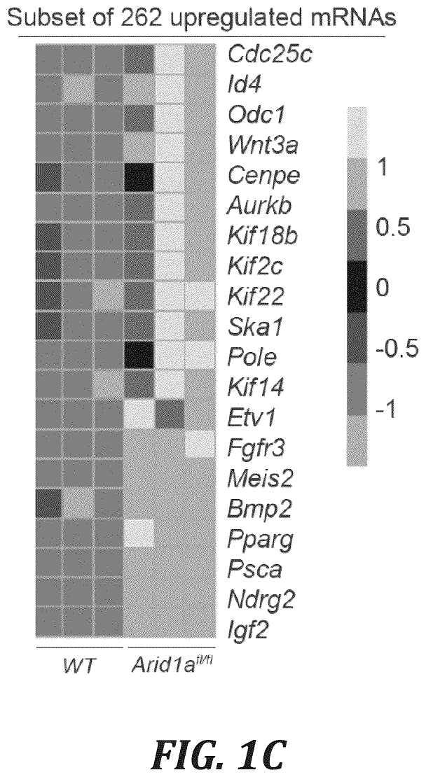

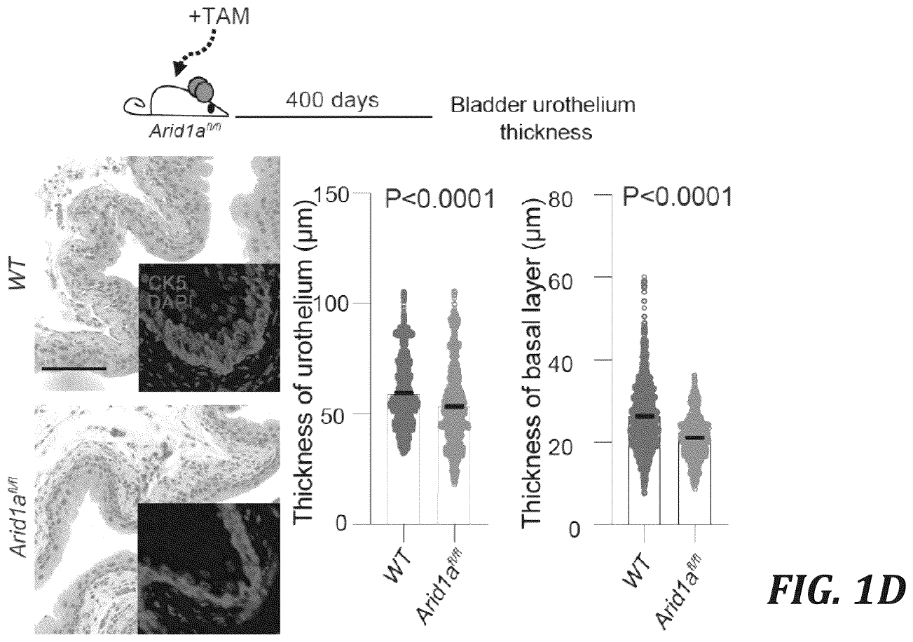

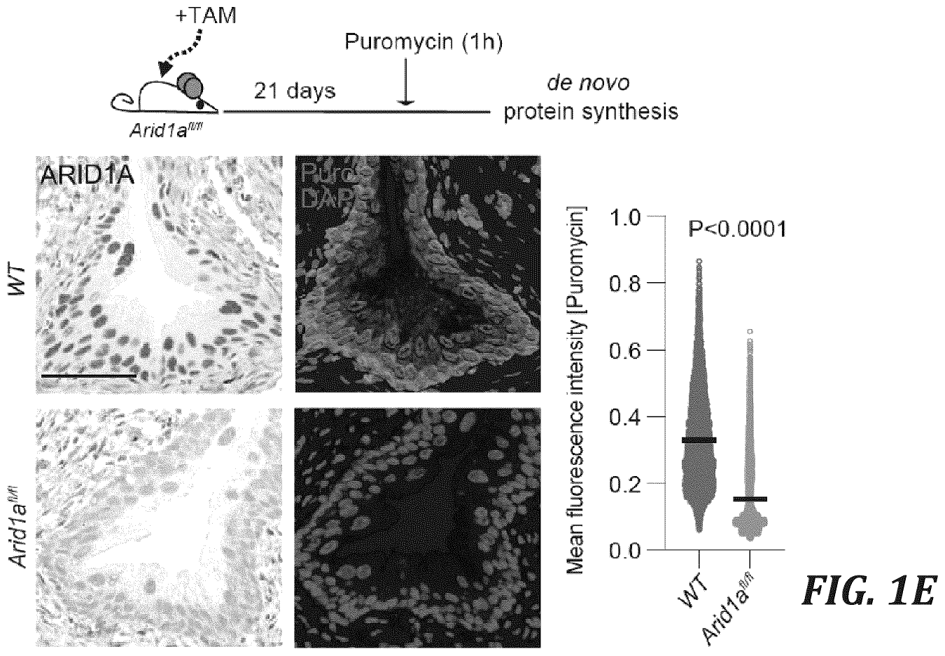

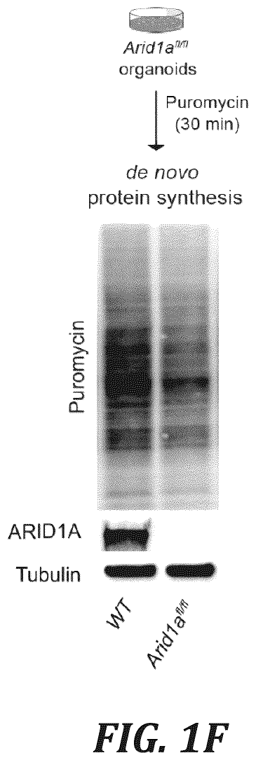

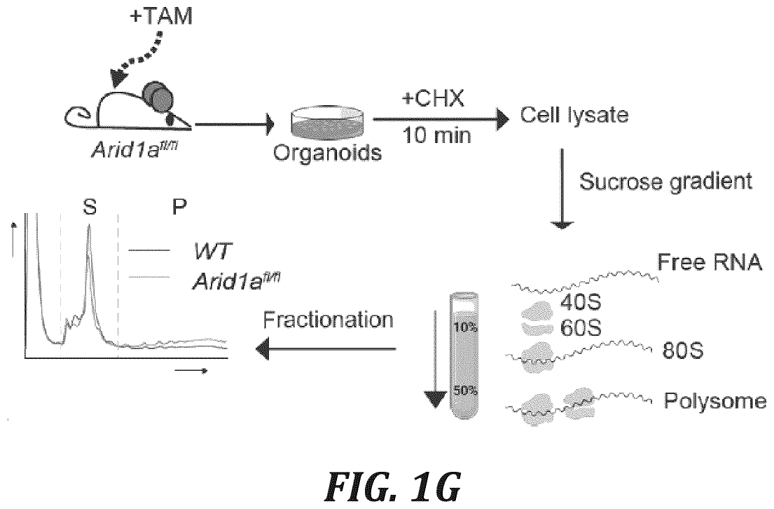

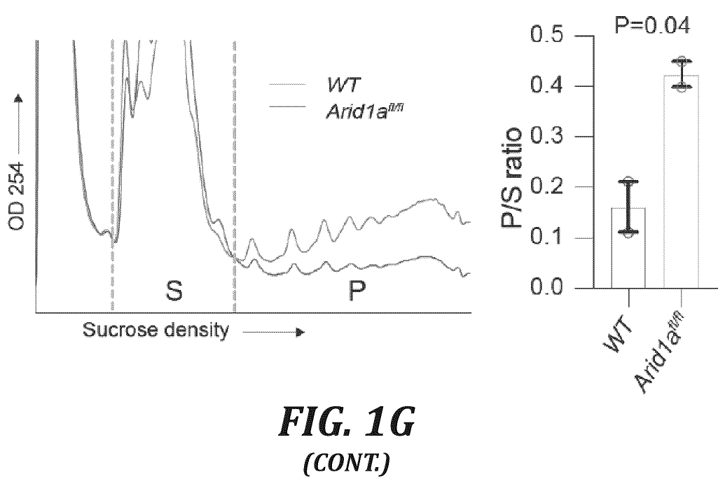

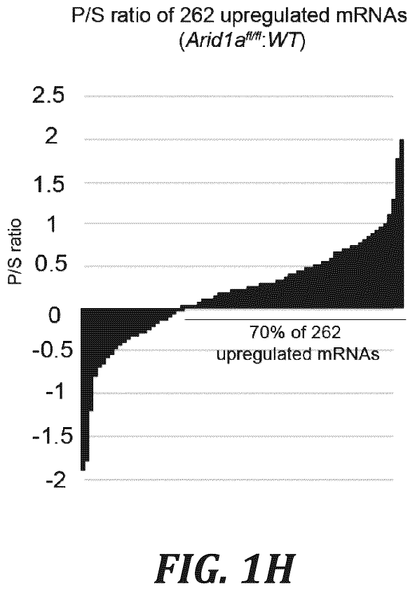

To study the physiologic impact of deleting ARID1A on gene expression, a UBC-Cre^{ERT2}; ROSA-LSL-Yfp^{KI/KI};Arid1a^{LoxP/LoxP} mouse model (herein referred to as Arid1a^{fl/fl}) in which the administration of tamoxifen leads to deletion of ARID1A and expression of YFP in multiple tissues (FIG. 1A) was used. Within the urothelium, a recombination efficiency of 83% was observed. The urothelium was chosen for these studies because bladder cancer is the 6^{th} most common malignancy in humans and 25% of urothelial carcinoma patients exhibit ARID1A loss-of-function mutations. Given that ARID1A loss has been shown to increase transcript levels of oncogenic genes in other tissues, it was sought to determine if the same was true within urothelial cells.

Basal cells, a cell of origin of invasive urothelial malignancies were isolated from wild-type (WT) and Arid1a^{fl/fl} bladders and subjected to RNA-seq. 262 upregulated mRNAs were observed in Arid1a^{fl/fl} cells and 80% of these were direct targets of ARID1A. Most overexpressed mRNAs were classified as regulators of cell proliferation (FIG. 1B). Furthermore, these genes included Aurora Kinase B (Aurkb), Insulin-Like Growth Factor 2 (Igf2), Fibroblast Growth Factor Receptor 3 (Fgfr3), and Ornithine Decarboxylase 1 (Odc1), all of which are cancer drivers (FIG. 1C). Overexpression of Aurkb, Igf2, or Fgfr3 in transgenic models is sufficient to drive uncontrolled cell growth and tumor formation. Given these findings, Arid1a^{fl/fl} mice were aged for 400 days to determine if they develop bladder tumors. Surprisingly, despite the transcriptional activation of these gene networks, Arid1a^{fl/fl} mice exhibited a decrease in urothelial thickness characterized by fewer basal cells, and 0/9 mice developed tumors (FIG. 1D).

These observations raised the critical question of whether ARID1A simultaneously controls protein synthesis. To measure protein synthesis rates in vivo a puromycin incorporation assay was conducted and a 60% decrease in de novo protein synthesis was observed in ARID1A-deficient urothelium (FIG. 1E). An inducible K5-Cre^{ERT2};ROSA-LSL-Yfp^{KI/KI};Arid1a^{LoxP/LoxP} model (referred to as K5 ARID1A^{fl/fl}) with 81-87% recombination efficiency in basal cells was generated and the same reduction in protein synthesis with striking cell type specificity was observed. To determine if this decrease was cell autonomous, protein synthesis rates in organoids grown from Arid1a^{fl/fl} basal urothelium was measured. Puromycin incorporation and ³⁵S-methionine incorporation were decreased in ARID1A-deficient organoids (FIG. 1F).

To determine whether ARID1A loss while increasing a distinct network of pro-proliferative and potentially oncogenic network of mRNAs (FIG. 1C), simultaneously caused widespread transcriptional attenuation that would result in the perceived decreased protein synthesis, a RNA-seq of spike-in normalized WT and Arid1a^{fl/fl} basal urothelial organoids, was conducted. No widespread decrease in mRNA levels across the transcriptome was observed. Lastly, upon adding back ARID1A in ARID1A-deficient organoids protein synthesis rates were restored. These findings demonstrate that ARID1A loss leads to opposing effects on transcription and translation which is associated with an absence of tumor formation.

Next, the impact of the decreased de novo protein synthesis on ribosome abundance on mRNA was determined by polysome analysis. It was observed that Arid1a^{fl/fl} urothelial cells exhibited a significant increase in poly-ribosome abundance normalized to the sub-polysomal fraction (P/S ratio) (FIG. 1G). Increased P/S ratio in the context of decreased protein synthesis can be caused by slower elongation rates. To determine if ARID1A loss slows down translation elongation, the ribosome half transit time was measured. A half transit time of 85.5 seconds (+/−13 seconds) in WT organoids and 143 seconds (+/−42 seconds) in Arid1a^{fl/fl} organoids was observed supporting the idea that protein synthesis is disrupted at the level of elongation.

These results raised the question of how decreased ribosome transit time impacts the translation of distinct mRNA species, particularly the 262 mRNA that were upregulated upon loss of ARID1A (FIG. 1C). Ribosome bound mRNA was sequenced to measure P/S ratios transcriptome-wide. Strikingly, 70% of the 262 upregulated mRNAs in Arid1a^{fl/fl} cells exhibited an increase in P/S ratio providing genome level evidence of a conflict between transcription of these genes and their translation (FIG. 1H). Likewise, using tandem-mass-tag (TMT) mass spectrometry and immunoblot analysis, 70% of the upregulated mRNAs were observed to show no increase in protein levels (FIG. 1I). These conflicted genes were enriched for guanine and cytosine nucleotides within their coding sequences (CDS), which was consistent with previous findings that higher guanine and cytosine content in the CDS is associated with slower codons. Thus, loss of ARID1A prevents the efficient translation of the majority of upregulated mRNAs leading to transcriptional-translational conflict.

### Example 2

ARID1A is a Central Regulator of Eukaryotic Elongation Factor 2 (eEF2)

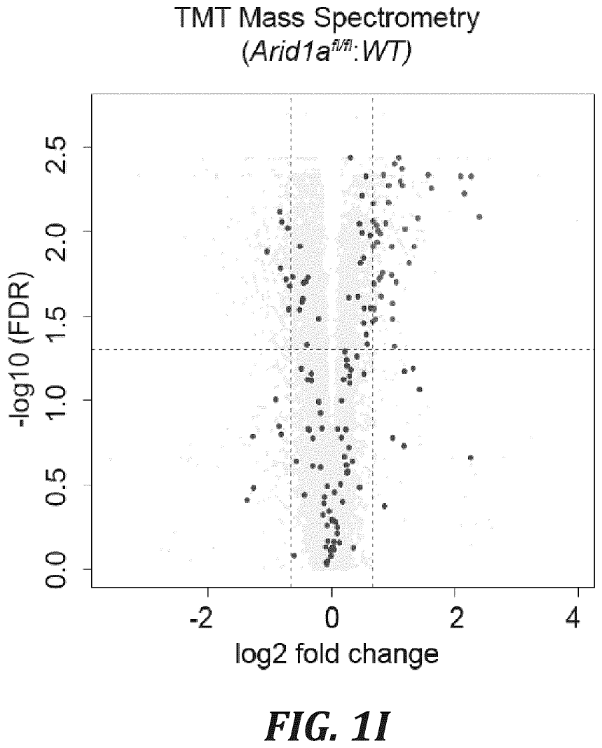

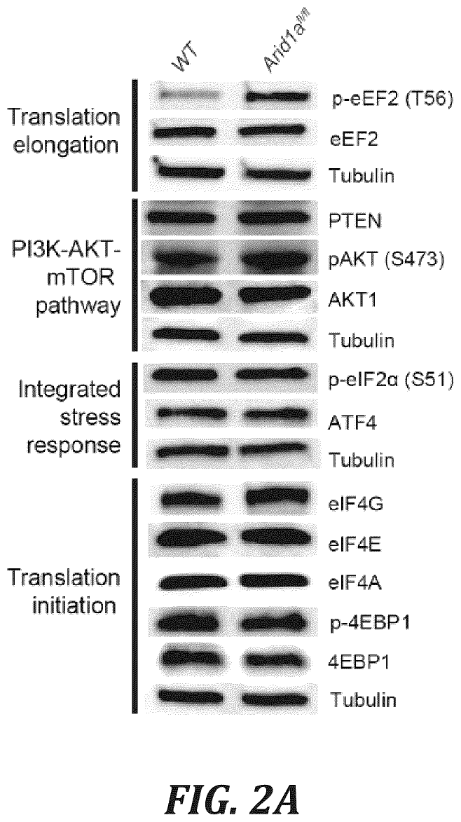

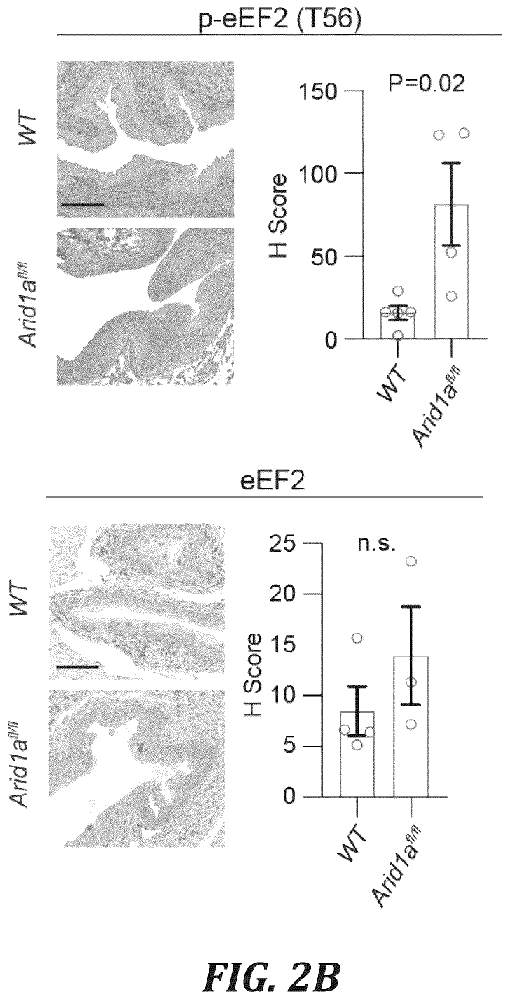

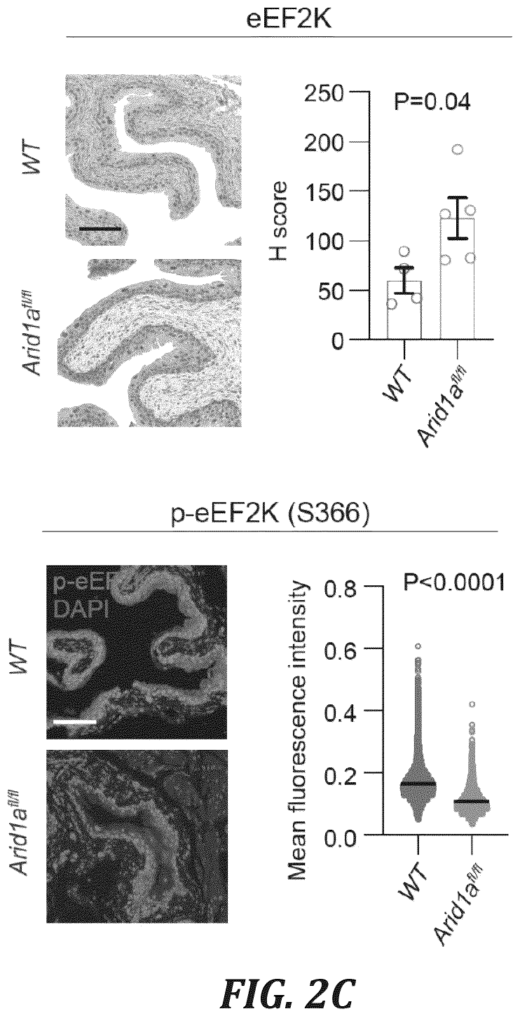

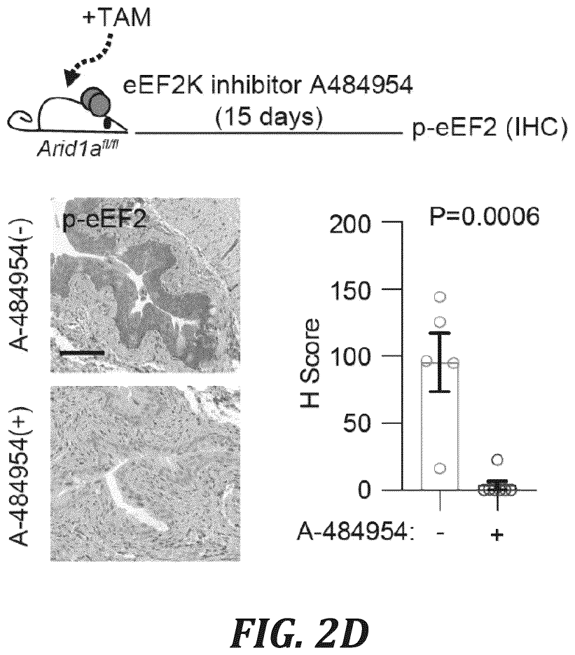

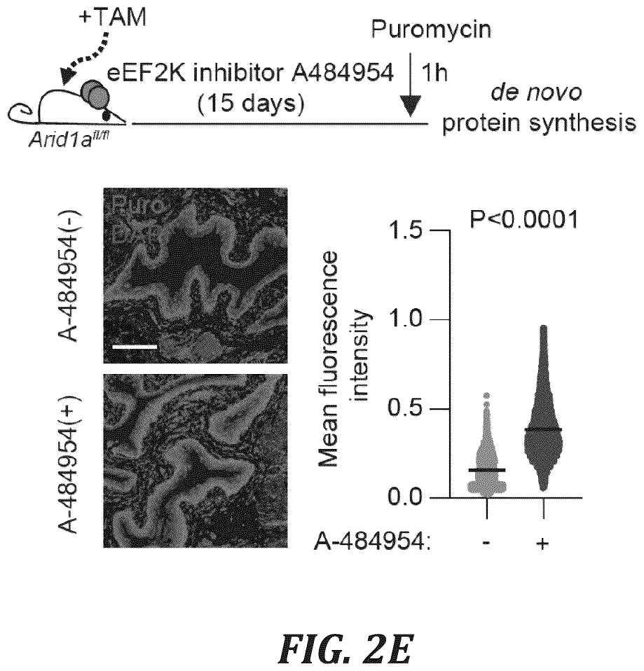

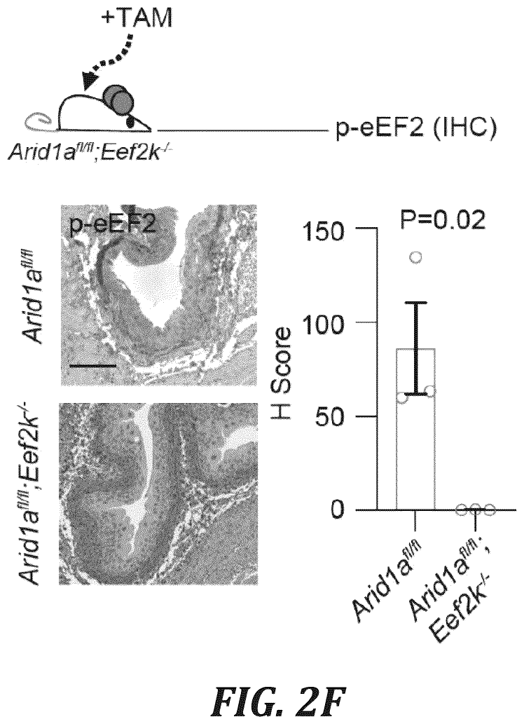

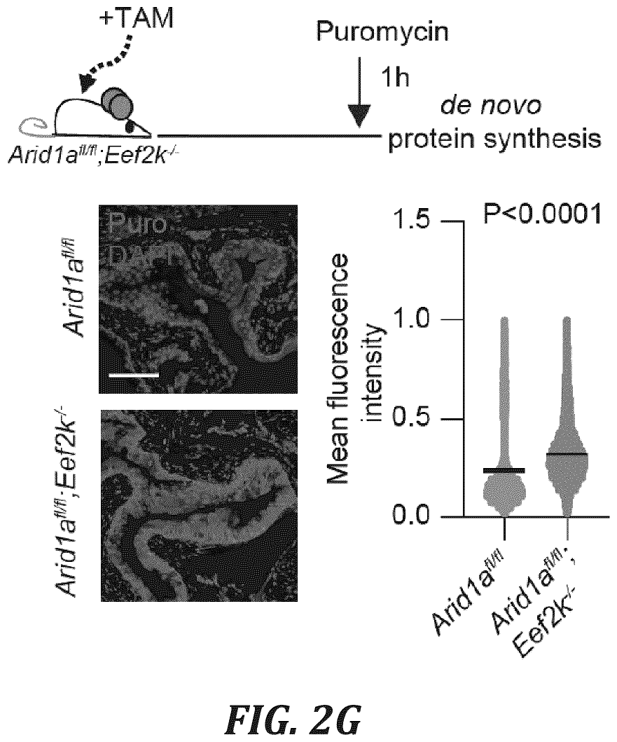

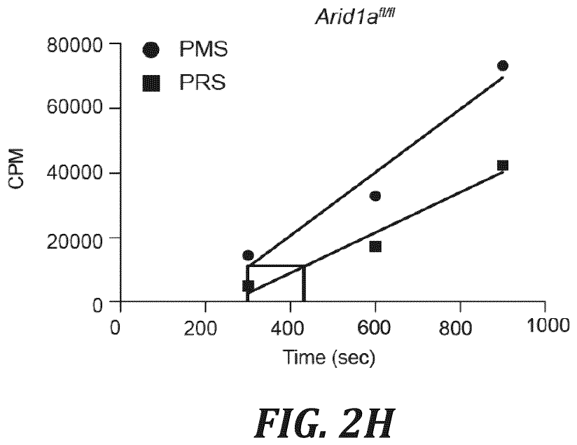

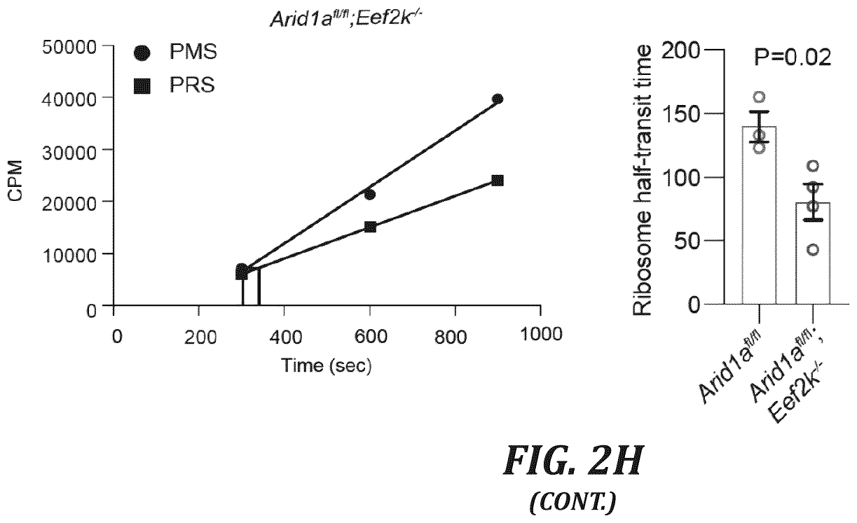

Next, the mechanism by which ARID1A regulates translation elongation was determined by conducting a candidate gene analysis of regulators of protein synthesis. No differences in 5.8S and 5S rRNA levels were observed. Next, the PI3K signaling pathway (PTEN and AKT (total and phospho-5473)), the integrated stress response (phospho-eIF2α S51 and ATF4), the eIF4F translation initiation complex (4EBP1 (total and phospho-T37/46), eIF4G, eIF4E, and eIF4A), and translation elongation regulators (eEF2 (total and phospho-T56)) were investigated. Of these, only eEF2 phosphorylation was significantly increased upon ARID1A loss in vitro and in vivo (FIGS. 2A-2B). eEF2 catalyzes GTP-dependent ribosome translocation during translation elongation and its phosphorylation at T56 decreases protein synthesis rates. The primary kinase responsible for eEF2 phosphorylation is the eukaryotic elongation factor 2 kinase (eEF2K) which regulates the cellular response to nutrient deprivation. eEF2K mRNA and protein levels were measured and both were observed to be increased in Arid1a^{fl/fl} urothelium, coinciding with a significant reduction in its inactive S366 phosphorylated form (FIG. 2C).

A pharmacogenetic strategy was utilized to determine if eEF2K activity regulated protein synthesis and elongation speed in Arid1a^{fl/fl} urothelium. First, Arid1a^{fl/fl} mice were treated with the eEF2K small molecule inhibitor A-484954. An attenuation of eEF2 phosphorylation concomitant to restoration of protein synthesis in Arid1a^{fl/fl} bladders was observed (FIGS. 2D-2E). Next, Arid1a^{fl/fl} and Eef2k^{-/-} mice were intercrossed to develop the Arid1a^{fl/fl};Eef2k^{-/-} mouse model where eEF2 can no longer be phosphorylated (FIG. 2F). The loss of Eef2k restored protein synthesis in ARID1A-deficient urothelium (FIG. 2G). Furthermore, loss of Eef2k restored translation elongation rates in Arid1a^{fl/fl};Eef2k^{-/-} organoids back to WT levels in a cell autonomous manner (ribosome half transit time of 80.2 seconds (+/−28 seconds)) (FIG. 2H). These data show that ARID1A maintained mRNA translation elongation by promoting eEF2 activity through downregulation of eEF2K. However, in the context of ARID1A loss, eEF2 activity decreased leading to slower ribosome transit time and a conflict between upregulated transcripts and their subsequent translation.

### Example 3

RASGRP1 is Necessary to Maintain eEF2 Activity

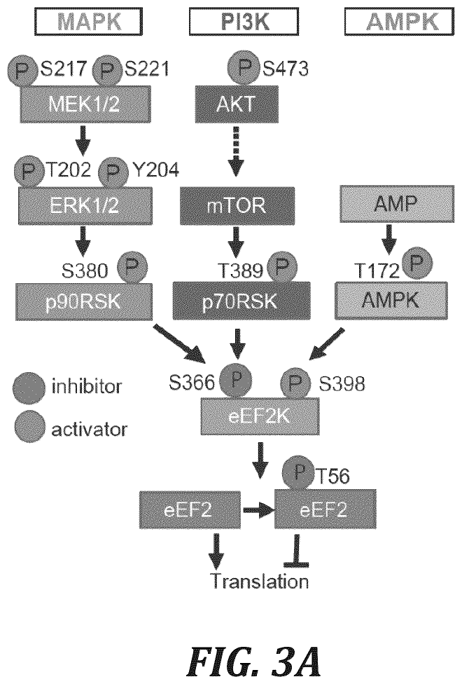

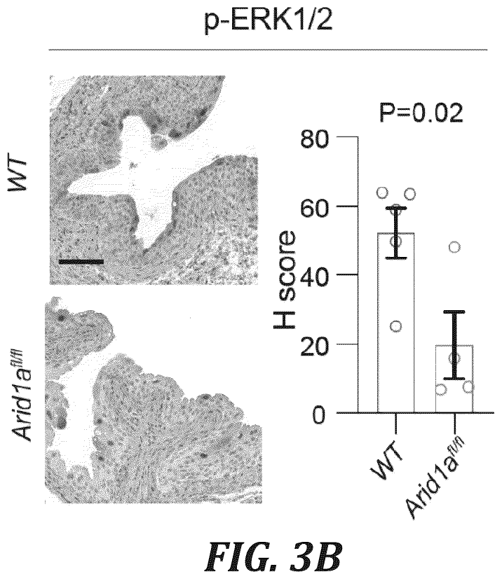

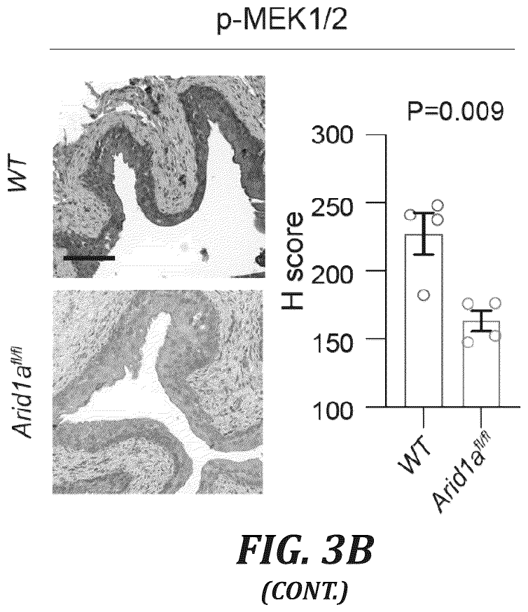

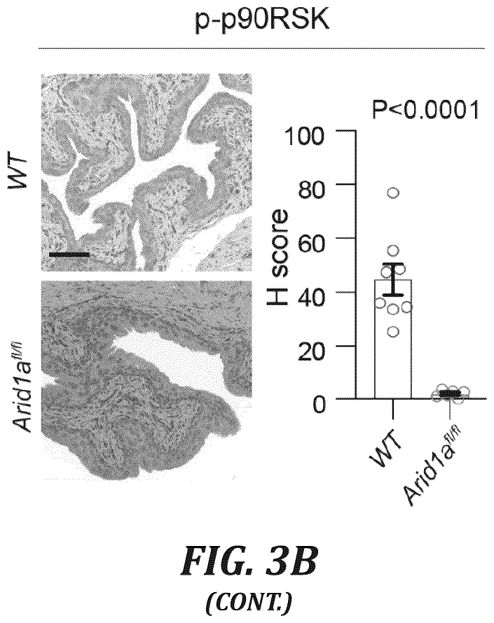

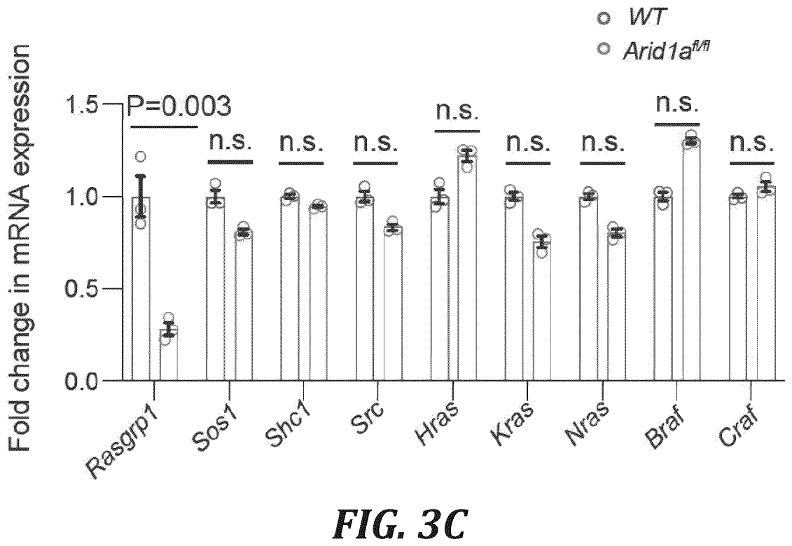

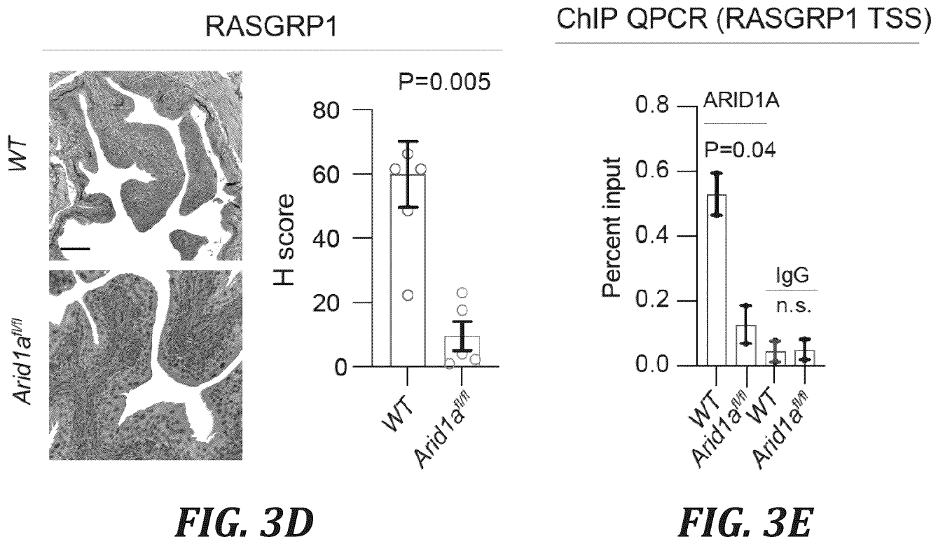

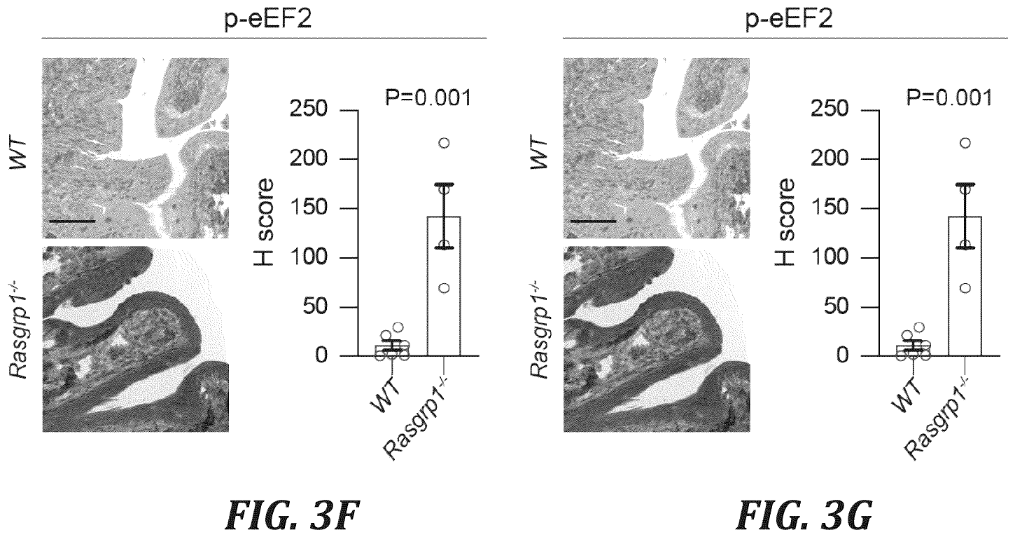

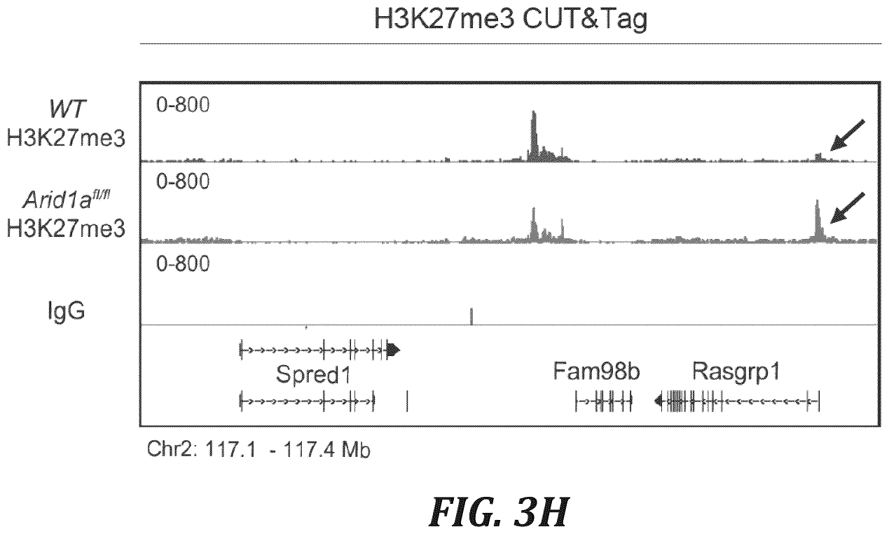

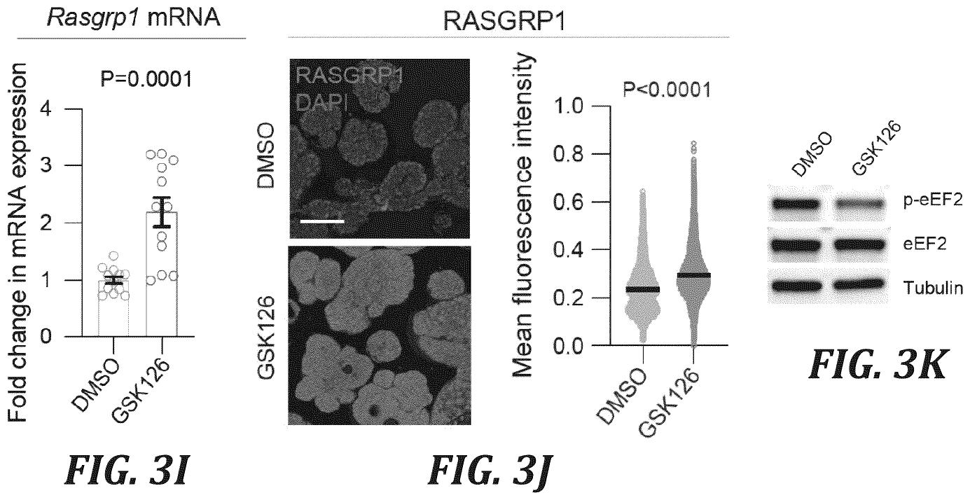

Next, the mechanism by which ARID1A negatively regulated eEF2K activity was sought to be elucidated. The current data demonstrated that ARID1A-deficiency led to a decrease in eEF2K phosphorylation at serine 366 (FIG. 2C). The decrease of this inhibitory post-translational modification of eEF2K led to an increase in eEF2 phosphorylation. The MAP kinase (MAPK) and PI3K-AKT-mTOR signaling pathways converge on eEF2K S366 (FIG. 3A). In addition, an AMP kinase (AMPK) activating eEF2K phosphorylation site at serine 398 has also been described. It was observed that ARID1A loss in urothelial organoids did not decrease PI3K-AKT-mTOR signaling or increase AMPK activity, suggesting these pathways were not responsible for the increase in eEF2 phosphorylation caused by ARID1A deficiency (FIG. 2A). However, MEK1/2 (S217/221), ERK (T202/Y204), and p90RSK (S380) phosphorylation levels decreased in ARID1A-deficient urothelium without impacting total protein abundance or transcript levels (FIG. 3B). This finding suggested that the MAPK pathway is downregulated in ARID1A-deficient bladder urothelium which may impact eEF2K activity.

To determine how ARID1A controls the MAPK pathway, mRNA abundance of MAPK regulators in WT and Arid1a^{fl/fl} urothelial organoids were measured. Surveying components in the Ras-MAPK pathway, no difference in expression of Hras, Kras, Nras, Shc1, Src, Braf, Craf, or Sos1 was found (FIG. 3C). However, Rasgrp1 transcript levels were decreased by 80% and RASGRP1 protein abundance was also decreased in ARID1A-deficient urothelium (FIGS. 3C-3D). Furthermore, ARID1A bound to the Rasgrp1 promoter in WT urothelial cells (FIG. 3E), and Rasgrp1 mRNA and protein levels were restored by adding back ARID1A in Arid1a^{fl/fl} organoids. RASGRP1 is a diacylglycerol-regulated RasGEF, which activates the MAPK pathway with functions in T cells, T cell leukemia, and the intestinal epithelium. However, its role in the urothelium is unknown. To determine if the decrease in RASGRP1 can increase eEF2K activity, eEF2 phosphorylation in Rasgrp1^{-/-} urothelium was determined. Strikingly, Rasgrp1^{-/-} urothelium exhibited a significant increase in eEF2 phosphorylation (FIGS. 3F-3G), independent of total eEF2 protein levels, which was consistent with Arid1a^{fl/fl} mice.

Next, ARID1A regulation of Rasgrp1 expression was delineated. CUT&Tag chromatin profiling of histone H3 trimethylation of lysine 27 (H3K27me3) was conducted because downregulation of SWI/SNF activity is associated with H3K27me3 accumulation and repression of distinct genes. 2558 gene promoters were observed to exhibit a greater than 2-fold increase in the H3K27me3 in Arid1a^{fl/fl} organoids. Moreover, on a gene-specific level, H3K27me3 at the Rasgrp1 promoter was enriched 4.5-fold in ARID1A-deficient urothelial organoids and decreased in ARID1A add-back organoids (FIG. 3H). Accumulation of H3K27me3 suggested that loss of ARID1A may have increased polycomb repressive complex 2 (PRC2) activity at the Rasgrp1 promoter. PRC2 marks H3K27me3 through its catalytic subunit the enhancer of zeste 2 polycomb repressive complex 2 (EZH2). To demonstrate that EZH2 catalytic activity was responsible for downregulation of Rasgrp1, Arid1a^{fl/fl} organoids were treated with GSK126, a selective EZH2 inhibitor. Pharmacologic inhibition of EZH2 decreased H3K27me3 on the Rasgrp1 promoter, which restored Rasgrp1 mRNA and protein levels in Arid1a^{fl/fl} cells (FIGS. 3I-3J). This resulted in a decrease in eEF2 phosphorylation (FIG. 3K). These findings provide a mechanism by which ARID1A controls translation elongation through modulating histone dynamics on the Rasgrp1 promoter.

### Example 4

### Transcriptional-Translational Conflict is a Tumor Suppressive Barrier in the Urothelium

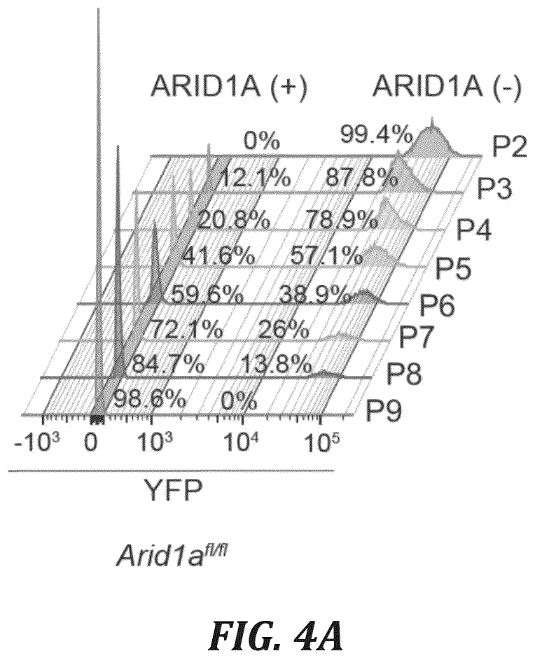

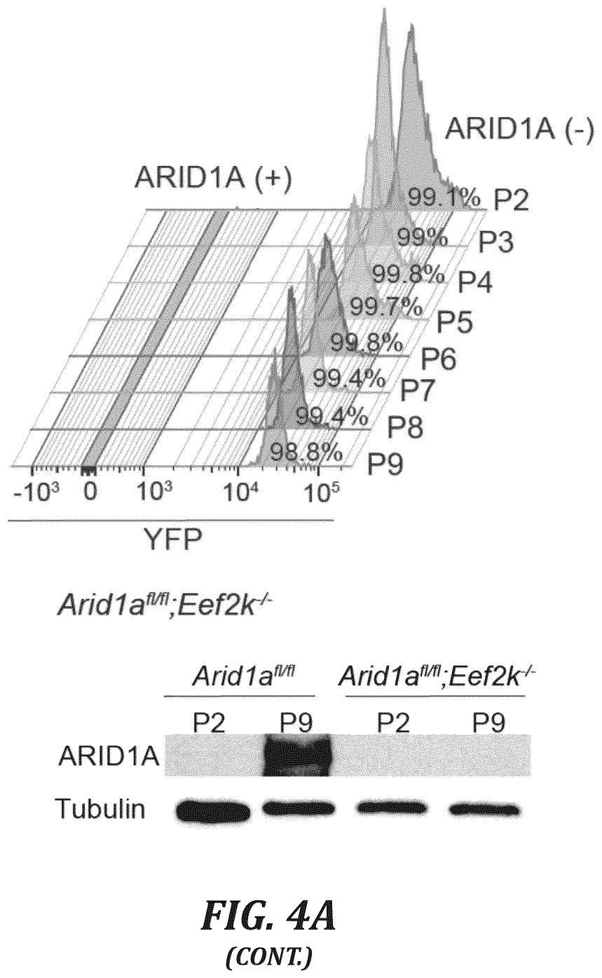

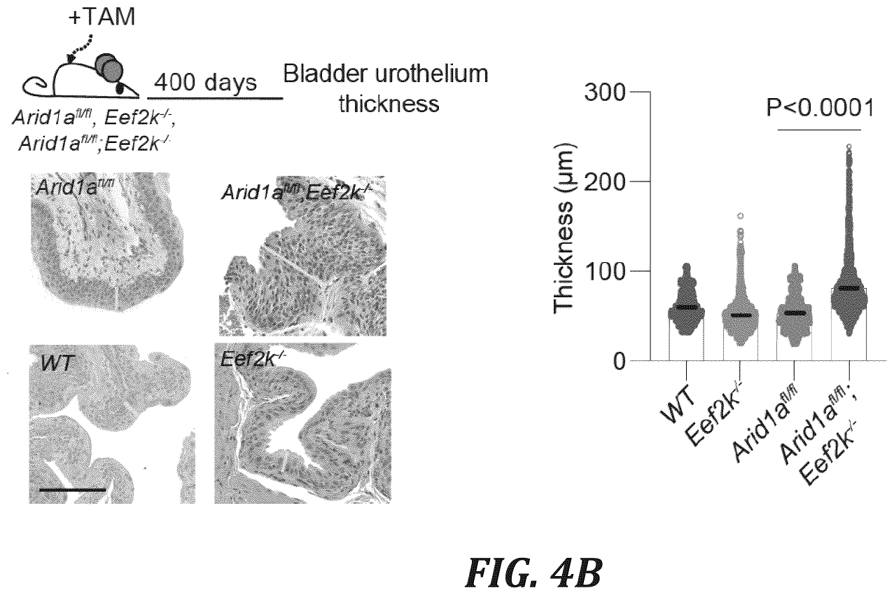

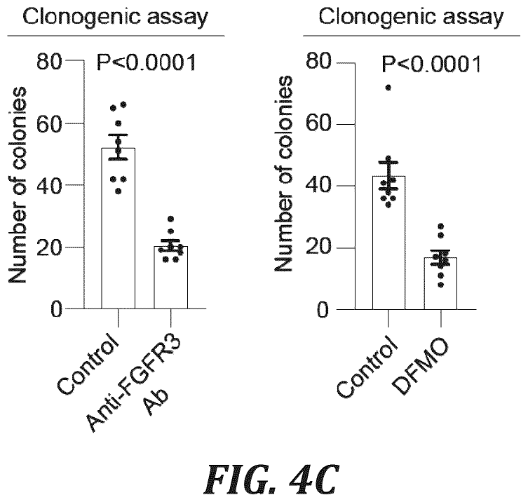

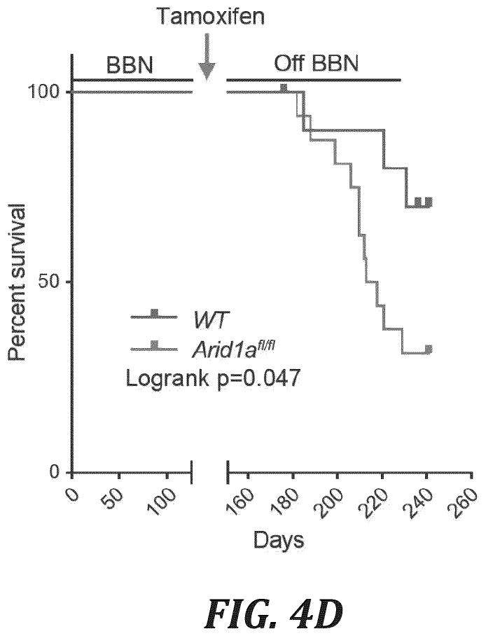

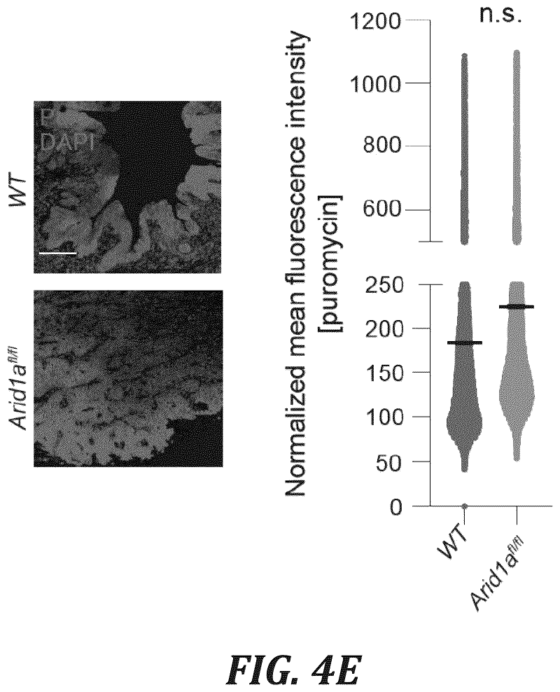

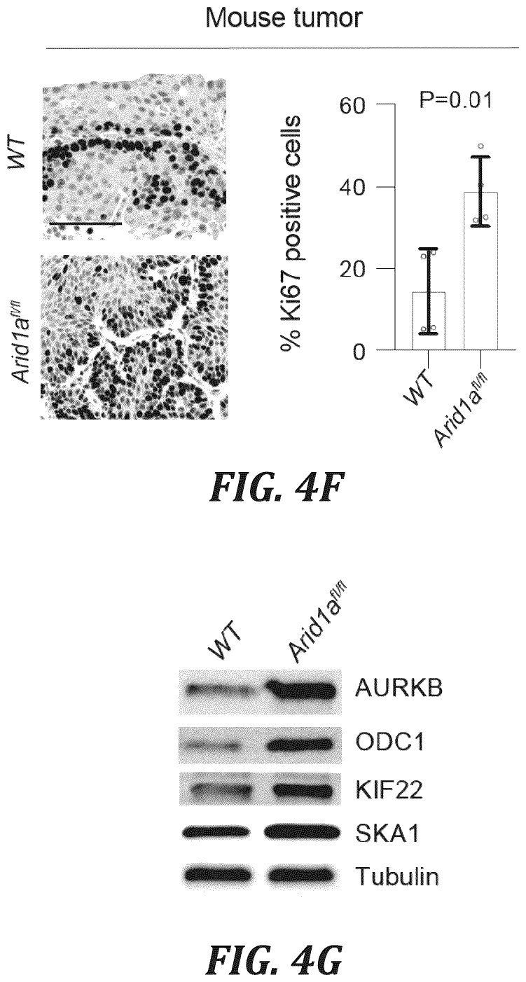

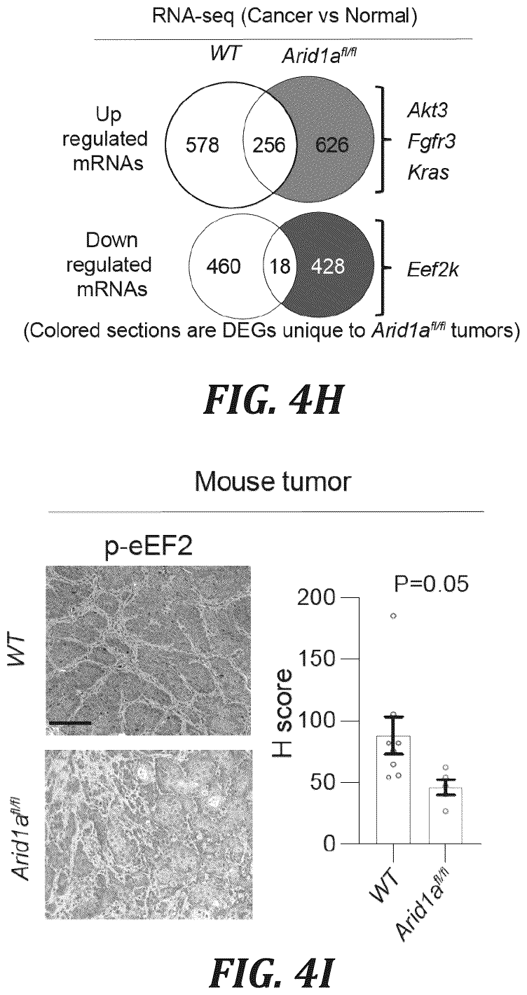

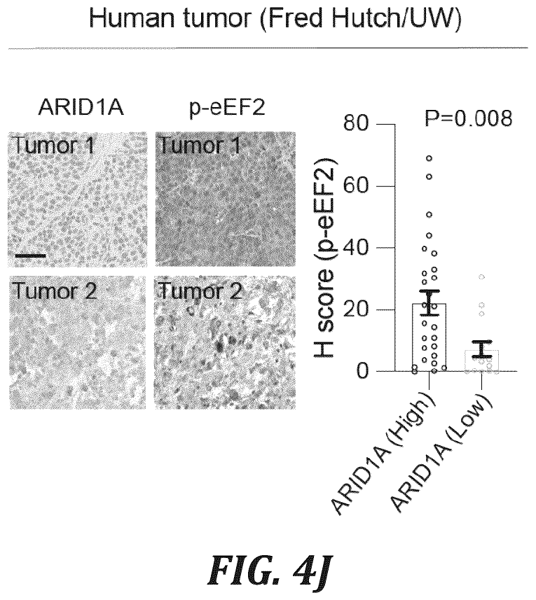

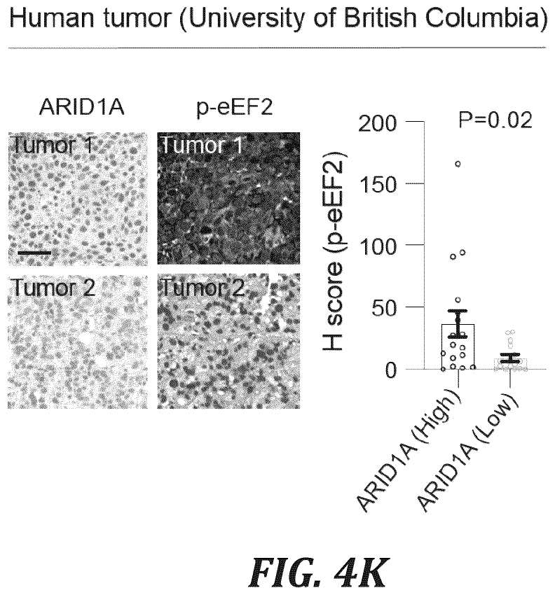

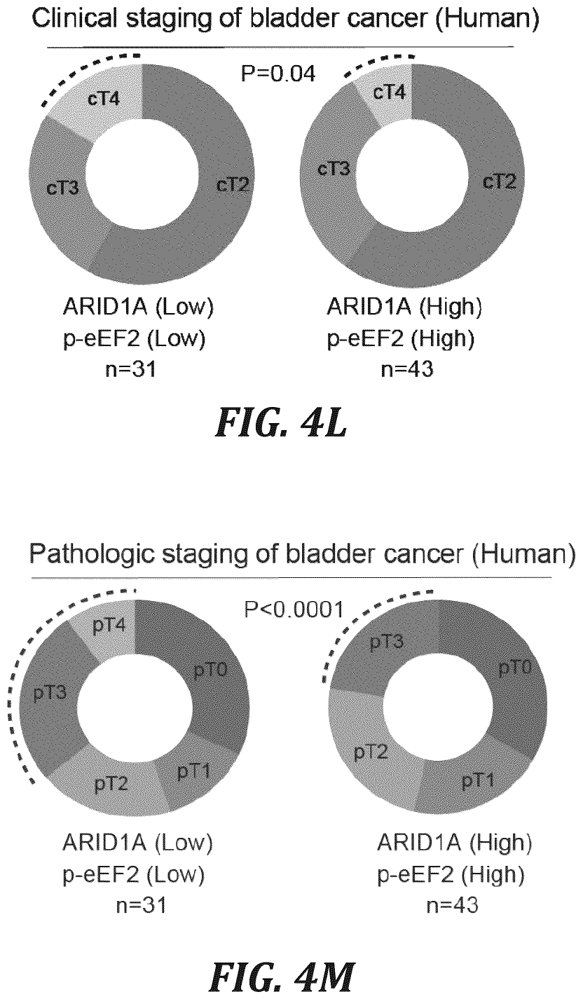

These findings raised the important question of how transcriptional-translational conflict is tumor suppressive in the urothelium. Transformation is a complex process that requires a cell to generate its own mitogenic signals, proliferate without limits, resist exogenous growth-inhibitory signals, evade apoptosis, and acquire vasculature. Successive passaging of primary Arid1a1^{fl/fl} urothelial organoids was observed to lead to the loss of ARID1A negative cells which was consistent with limited replicative potential (FIG. 4A). However, Arid1a^{fl/fl};Eef2k^{-/-} organoids remained ARID1A negative even after 9 passages, had higher clonogenic potential, and proliferated faster than Arid1a1^{fl/fl} cells (FIG. 4A). To determine if this aspect of transformation could be observed in vivo, Arid1a^{fl/fl};Eef2k^{-/-} mice were aged for 400 days. Remarkably, Arid1a^{fl/fl};Eef2k^{-/-}, but not WT, Arid1a^{fl/fl}, or Eef2k^{-/-} urothelium exhibited unusually thickened urothelium consistent with uncontrolled cell growth (FIG. 4B). To determine if this phenotype was mediated by de-repression of conflicted mRNA, Arid1a^{fl/fl};Eef2k^{-/-} cells were treated with either the ODC1 inhibitor difluoromethylornithine (DFMO) or an anti-FGFR3 antibody to target ODC1 or FGFR3, respectively, and clonogenic growth was measured. ODC1 and FGFR3 inhibition significantly decreased the uncontrolled growth of Arid1a^{fl/fl};Eef2k^{-/-} cells (FIG. 4C). These findings demonstrated that de-repression of conflicted mRNAs can unmask the oncogenic properties of ARID1A loss. Furthermore, they raised the question of the role of translation restoration in tumor progression.

To test this hypothesis, an experimental model was developed where one could first increase translation elongation rates while forming tumors, then delete ARID1A. One of the main carcinogens found in cigarettes is N-butyl-N-(4-hydroxybutyl)nitrosamine (BBN). BBN is a bladder tropic pro-carcinogen that is metabolized into BCPN (N-butyl-N-(3-carboxypropyl)nitrosamine) and concentrated in the bladder, which causes DNA damage and bladder tumors in mice, but does not cause cancer in other tissues. The process of carcinogenesis by BBN was observed to markedly increase eEF2 protein levels by 4-fold, with only a 2-fold change in eEF2 phosphorylation, and no change in eEF2K levels. This was associated with an increase in puromycin incorporation in bladder tumors compared to WT urothelium. WT and non-recombined Arid1a^{fl/fl} mice were treated with BBN for 18 weeks to form tumors first, then dosed with tamoxifen to delete Arid1a and monitored their survival (FIG. 4D). Arid1a^{fl/fl} mice developed larger tumors, succumbed to bladder cancer at a much higher rate than WT mice (68.7% versus 23% dead 100 days after last tamoxifen treatment, respectively), and maintained the increase in protein synthesis caused by carcinogenesis (FIGS. 4D-4E). Moreover, Arid1a^{fl/fl} bladder tumors proliferated faster than WT tumors (FIG. 4F). At a molecular level, previously conflicted mRNAs including Aurkb, Kif22, Odc1, and Ska1 were more efficiently translated in Arid1a^{fl/fl} tumors as compared to WT tumors (FIG. 4G).

Next, the drivers that increase protein synthesis to resolve transcriptional-translational conflict in ARID1A-deficient tumors were determined. A primary organoid line from a non-recombined BBN-induced Arid1a^{fl/fl} bladder tumor (WT tumor) was developed and used to generate an isogenic ARID1A-deficient line using lentiviral Cre recombinase. Pairwise RNA-seq analysis comparing normal to cancer urothelial cells in both the WT and Arid1a^{fl/fl} backgrounds was performed. The analysis was focused on up- or down-regulated mRNAs unique to Arid1a loss (FIG. 4H). Akt3, Fgfr1, and Kras were found to be upregulated in Arid1a^{fl/fl} tumors. These genes have been shown to increase mRNA translation initiation and/or elongation rates. The only protein synthesis regulator that was downregulated in Arid1a^{fl/fl} tumors was Eef2k (FIG. 4H). This was associated with a significant decrease in total eEF2K and eEF2 phosphorylation in Arid1a^{fl/fl} tumors (FIG. 4I). It has been shown that NF-kB is a negative regulator of Eef2k mRNA levels. NF-kB mRNA signature was determined in Arid1a^{fl/fl} tumors and high NF-kB activity compared to normal Arid1a^{fl/fl} cells was observed, suggesting a potential mechanism for down-regulation of eEFK2 mRNA.

These discoveries demonstrated that a specific threshold of protein synthesis must be achieved to overcome transcriptional-translational conflict and unlock the oncogenic potential of ARID1A loss. To determine if these findings can be observed in patients, ARID1A levels and eEF2 activity levels were measured in two independent international cohorts of muscle invasive bladder cancer. Patient tumors with low levels of ARID1A protein were more likely to have decreased eEF2 phosphorylation, an indication of increased eEF2 activity (FIGS. 4J-4K). Furthermore, ARID1A and phospho-eEF2 low patients were more likely to have high T-stage disease at diagnosis and even after neoadjuvant chemotherapy (FIGS. 4L-4M). Together, these findings mechanistically demonstrated how ARID1A is a context dependent tumor suppressor and raised the possibility that overcoming transcriptional-translational conflict through restored mRNA translation could be a linchpin to drive ARID1A-deficient tumor growth.

### Example 5

### Drug-Induced Transcriptional-Translational Conflict is a Therapeutic Vulnerability in ARID1A-Deficient Bladder Cancer.

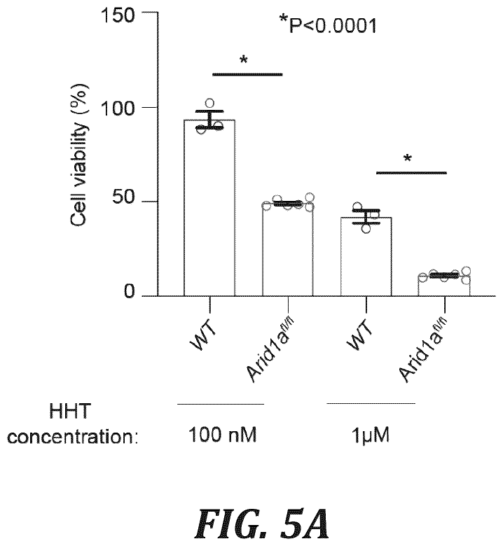

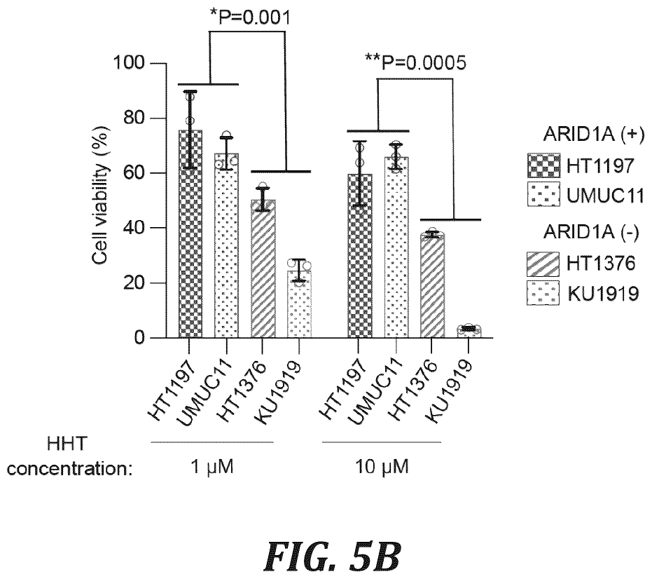

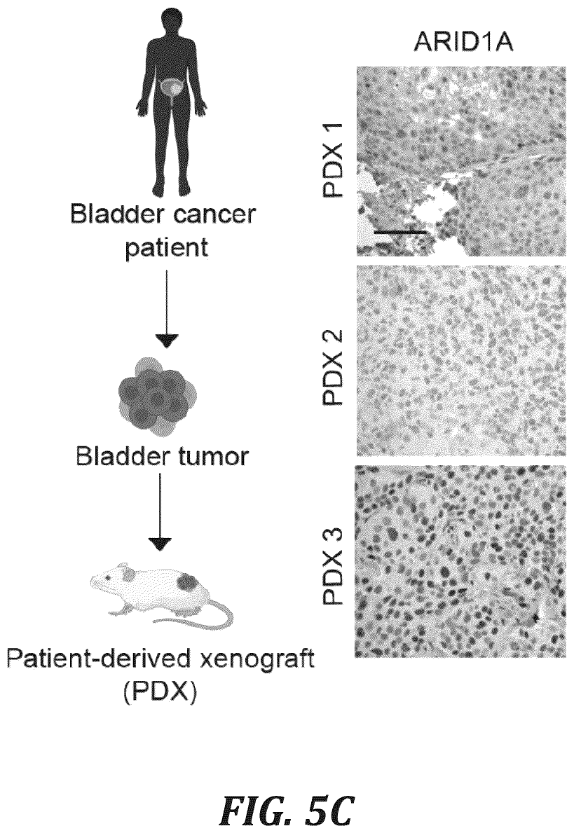

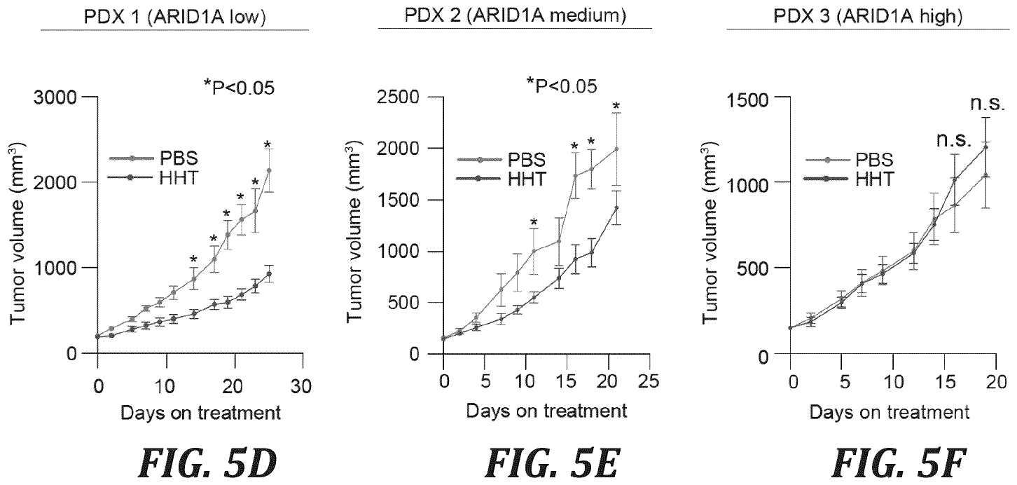

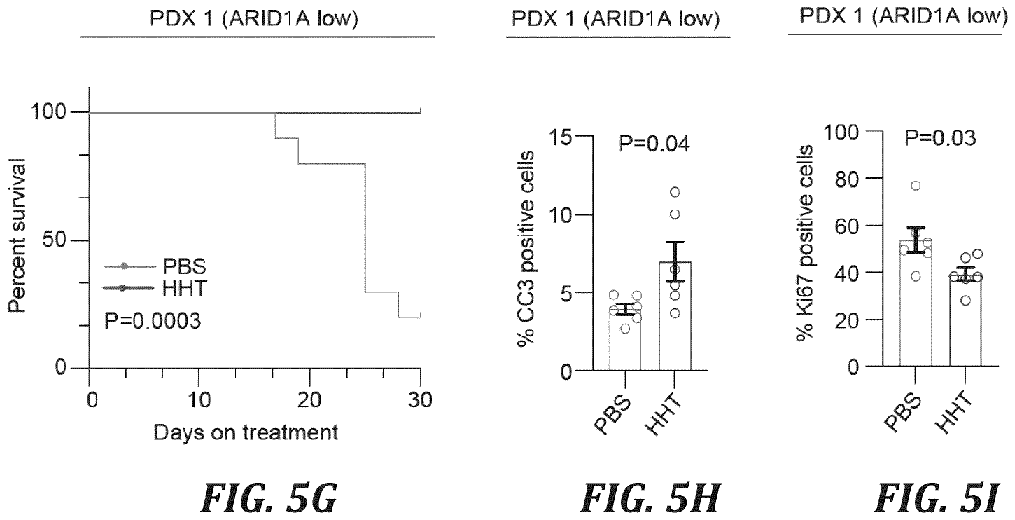

The data in the present disclosure demonstrated that transcriptional-translational conflict is a barrier to cancer progression. It is possible that inducing conflict by inhibiting protein synthesis may be a strategy to target cancer growth in genotypes that have overcome this barrier to thrive. To address this hypothesis, three human and murine models of bladder cancer were treated with the translation inhibitor homoharringtonine (HHT), which was chosen because it is FDA approved for refractory CML. HHT functions by preventing ribosomes from moving past the ATG start codon thereby stalling the initiation of elongation. First, isogenic WT and Arid1a^{fl/fl} BBN-induced cancer organoids were treated with HHT. Arid1a^{fl/fl} tumor organoids were sensitive to HHT at concentrations that had minimal impact on WT bladder cancer organoid growth (FIG. 5A). Next, 2 human ARID1A-deficient bladder cancer cell lines (HT1376 and KU1919) and 2 ARID1A-proficient bladder cancer cell lines (HT1197 and UMUC11) were treated with HHT. Interestingly, ARID1A null cell lines were significantly more sensitive to HHT compared to cell lines that expressed ARID1A (FIG. 5B).

Pre-clinical trials of patient derived xenografts (PDXs) that expressed low (PDX1), medium (PDX2), or high (PDX3) ARID1A protein levels (FIG. 5C), treated with HHT twice daily at a concentration physiologically achievable in human patients without any toxicity, were conducted. HHT decreased tumor growth by 59.3% in the ARID1A low model, but only decreased tumor growth by 35.9% in the ARID1A medium model (FIGS. 5D-5E). Strikingly, the ARID1A high model was completely insensitive to HHT (FIG. 5F). In addition, ARID1A low PDX exhibited the most significant improvement in survival when treated with HHT (FIG. 5G). At a cellular level, HHT increased apoptosis and decreased cell proliferation in the ARID1A-deficient, but not proficient PDXs (FIGS. 5H-5I). These findings demonstrated that translation inhibition could reverse the gains of cancers that overcome transcriptional-translational conflict and represented a new therapeutic paradigm.

### Example 6

eEF2 Phosphorylation LED to a Collateral Decrease in the Translation of DNA Damage Response Genes

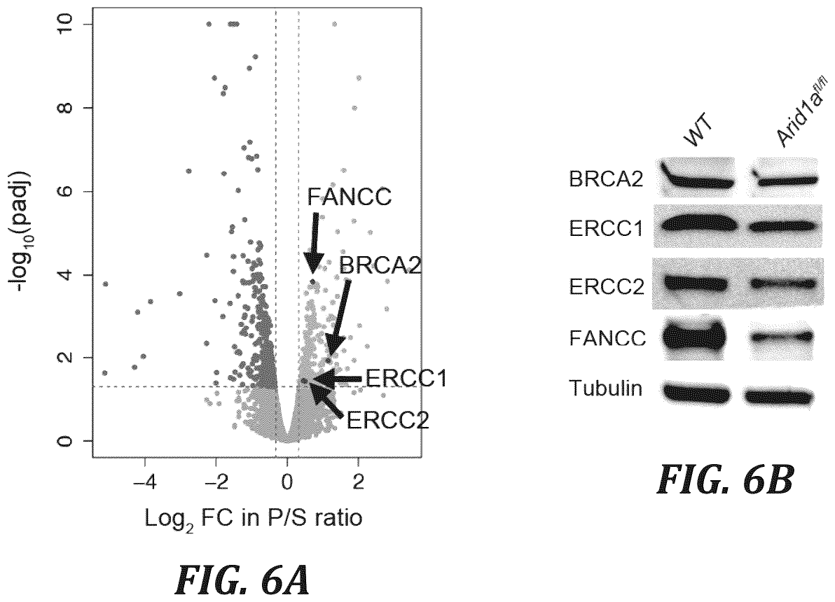

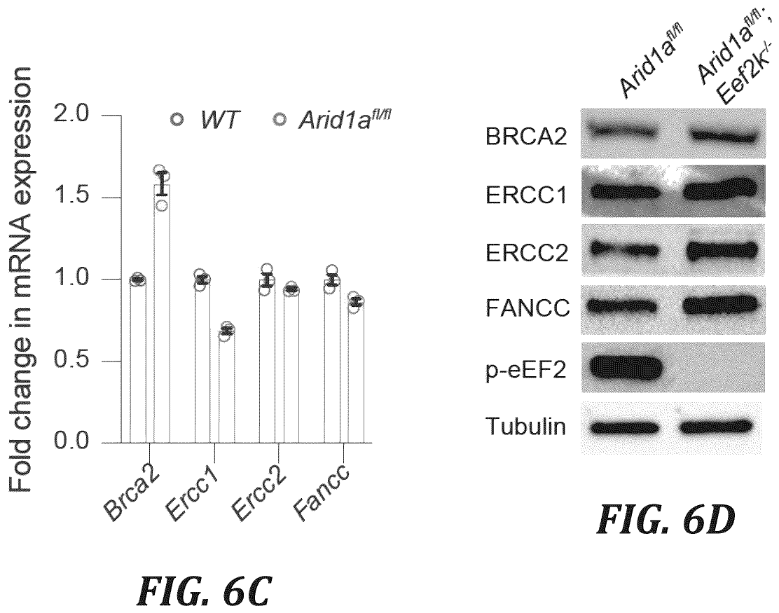

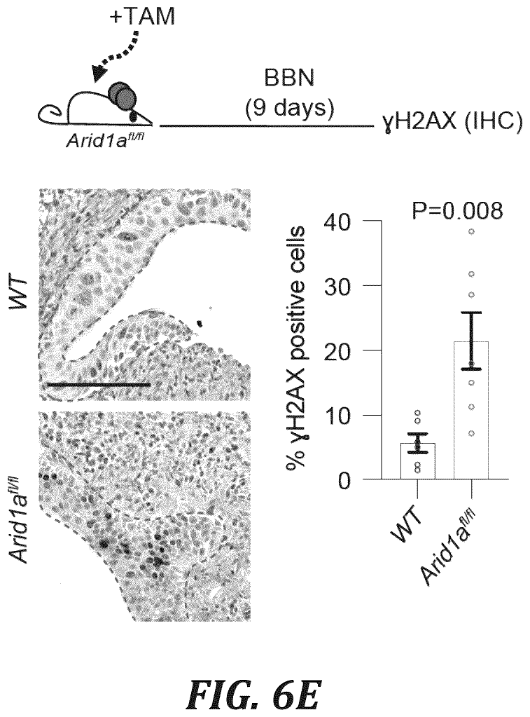

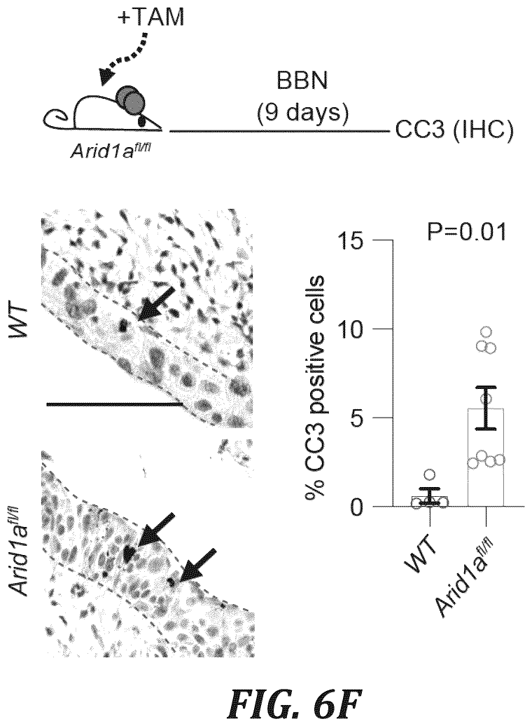

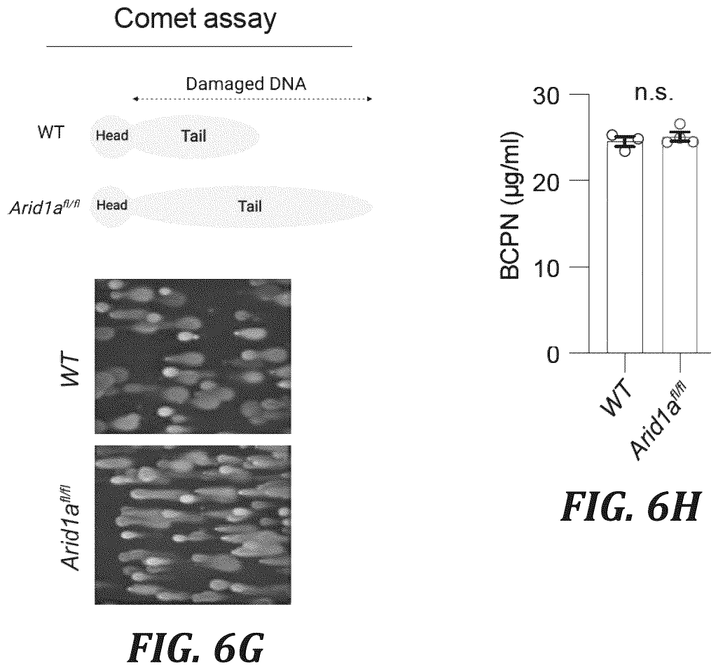

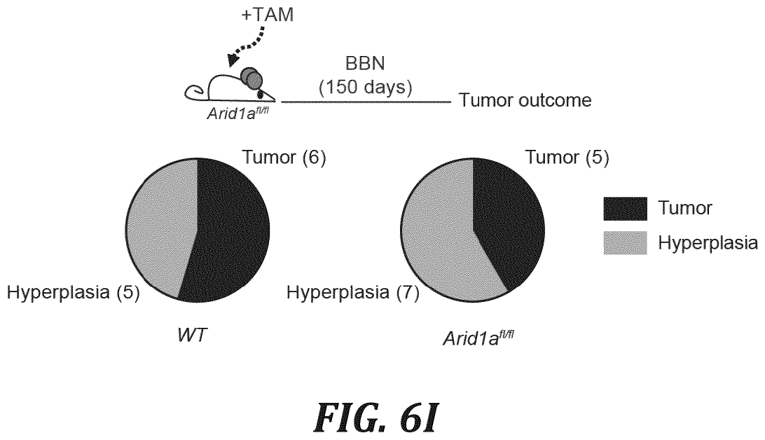

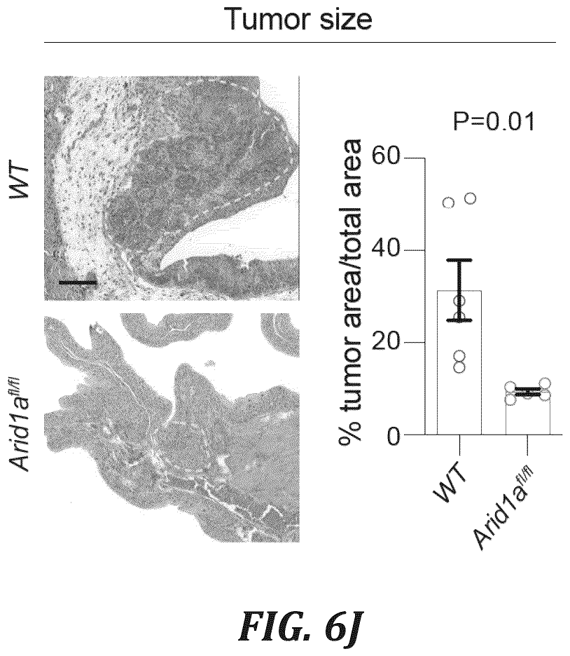

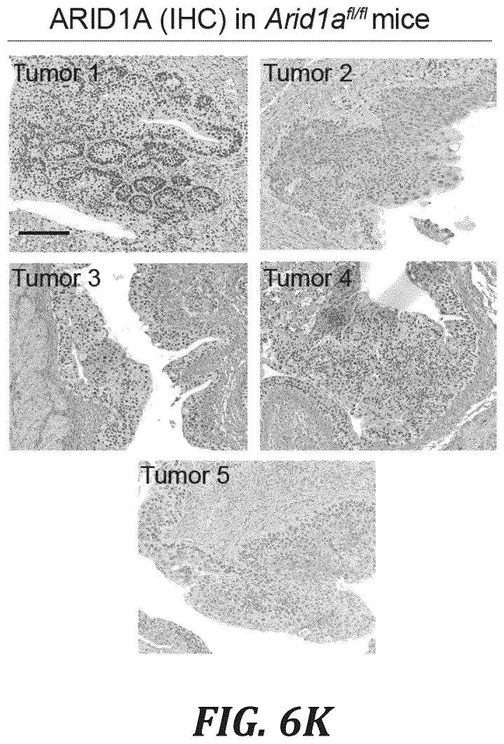

Transcriptional-translational conflict is a barrier to bladder cancer pathogenesis mediated by eEF2 phosphorylation. However, the effects of slower translation elongation caused by ARID1A loss likely impacts the protein synthesis of genes beyond the 262 upregulated oncogenic mRNAs. Indeed, upon analysis of mRNAs that exhibit no transcriptional difference, 278 additional stalled mRNAs in Arid1a^{fl/fl} organoids (>1.35-fold change in P/S ratio, FDR <0.05), were found (FIG. 6A). These stalled mRNAs also exhibited a high GC content within their coding sequences similar to the 262 upregulated mRNAs in Arid1a^{fl/fl} organoids. Interestingly, the top Reactome pathway was DNA double-strand break repair (FDR 5.02×10⁻⁴) and included genes such as BRCA2 DNA Repair-Associated Protein (Brca2), ERCC Excision Repair 1, Endonuclease Non-Catalytic Subunit (Ercc1), ERCC Excision Repair 2, TFIIH Core Complex Helicase Subunit (Ercc2), and FA Complementation Group C (Fancc) (FIG. 6A). These proteins maintain genome stability enabling DNA repair and cell survival in the context of DNA damage. BRCA2 plays a primary role in homology-directed repair of double-strand DNA breaks. ERCC1 and ERCC2 are critical for identification, excision, and sealing of damaged DNA through nucleotide excision repair. FANCC is a core component of the Fanconi Anemia pathway which is required to repair interstrand crosslinks in DNA. Protein levels of BRCA2, ERCC1, ERCC2, and FANCC were significantly decreased in Arid1a^{fl/fl} organoids without a concomitant change in mRNA levels (FIGS. 6B-6C). Furthermore, genetic de-repression of eEF2 phosphorylation restored the protein levels of each of these genes (FIG. 6D). Therefore, transcriptional-translational conflict leads to the collateral decrease of gene networks critical for DNA damage repair.

To determine if the decreased protein synthesis of DNA damage response (DDR) genes created a vulnerability to DNA damage in Arid1a-deficient urothelium, WT and Arid1a/lf mice were treated with BBN for 9 days and γH2AX foci (a marker for double stranded DNA breaks) and cleaved caspase 3 (CC3, a marker for apoptosis) were measured. Arid1a-deficient mice exhibited a significant increase in urothelial γH2AX and CC3 (FIGS. 6E-6F). This was associated with increased DNA damage as measured by the comet assay (FIG. 6G). Mass spectrometry for BCPN in WT and Arid1a^{fl/fl} mouse urine showed no difference in carcinogen exposure (FIG. 6H). These findings suggested that Arid1a^{fl/fl} cells could exhibit impaired transformation due to an inability to efficiently repair damaged DNA. To test this concept, WT and Arid1a-deficient mice were dosed with BBN for 150 days. While both WT and Arid1a^{fl/fl} mice developed the same number of tumors, Arid1a^{fl/fl} tumors were smaller compared to their WT counterparts (FIGS. 6I-6J). It was rationalized, given the mosaic nature of the Arid1a^{fl/fl} model, if loss of ARID1A is an impediment towards transformation, then the WT urothelium should more readily form tumors. Indeed, all tumors in Arid1a^{fl/fl} mice still expressed ARID1A (FIG. 6K). Thus, ARID1A is necessary to enable the survival of BBN-treated urothelium which raised the question of the role of translation elongation in this process.

### Example 7

eEF2 Activity is Necessary for Carcinogenesis in the Context of ARID1A Loss

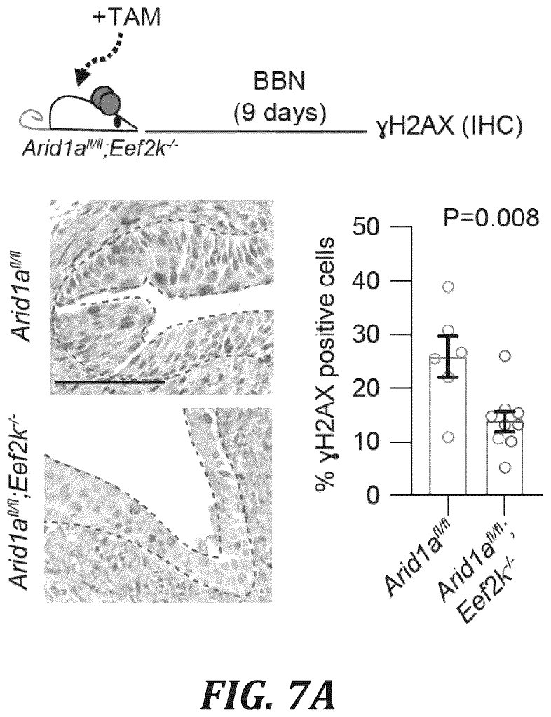

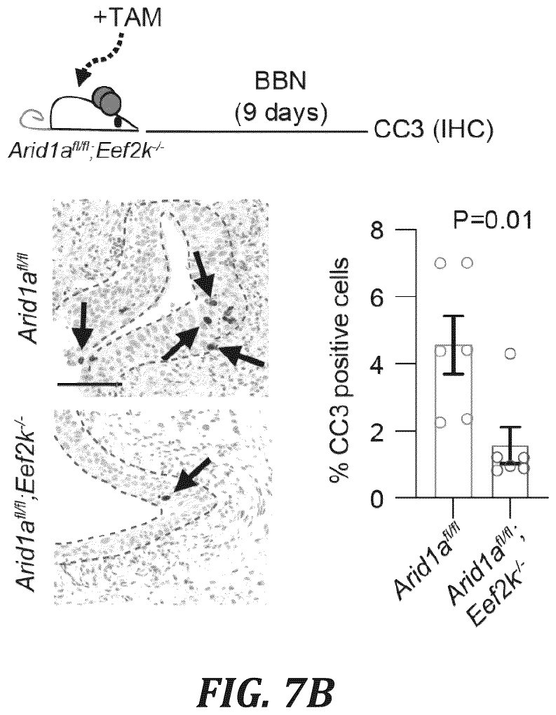

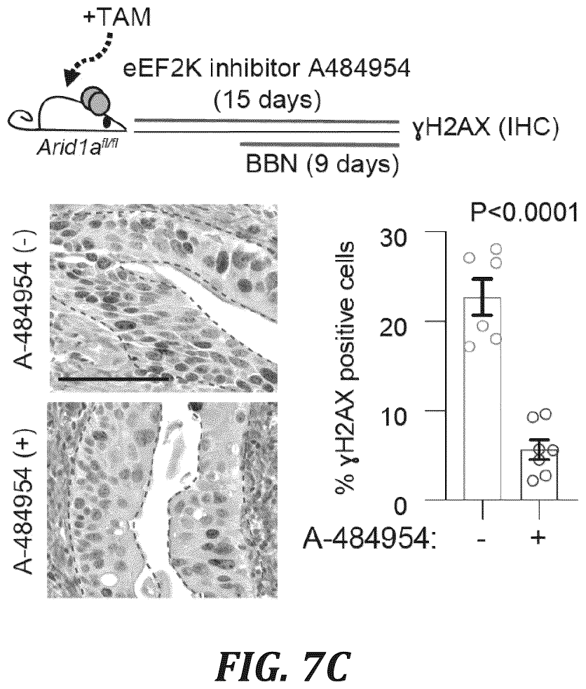

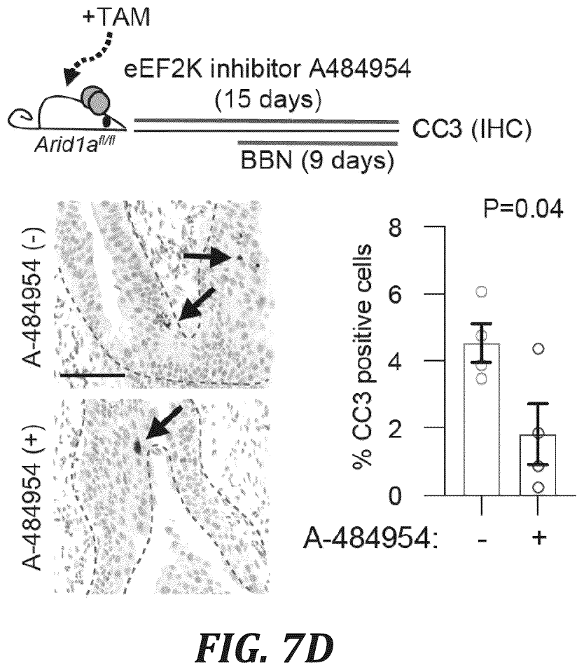

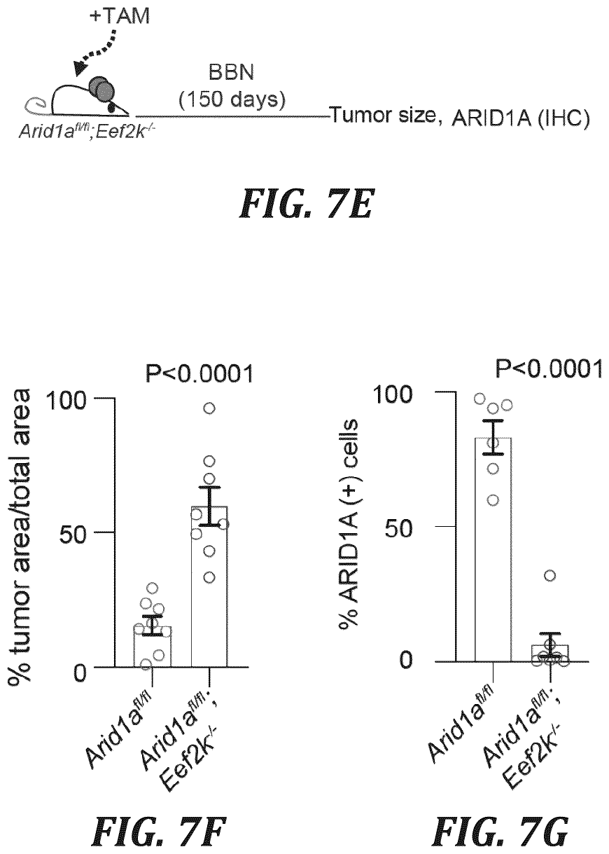

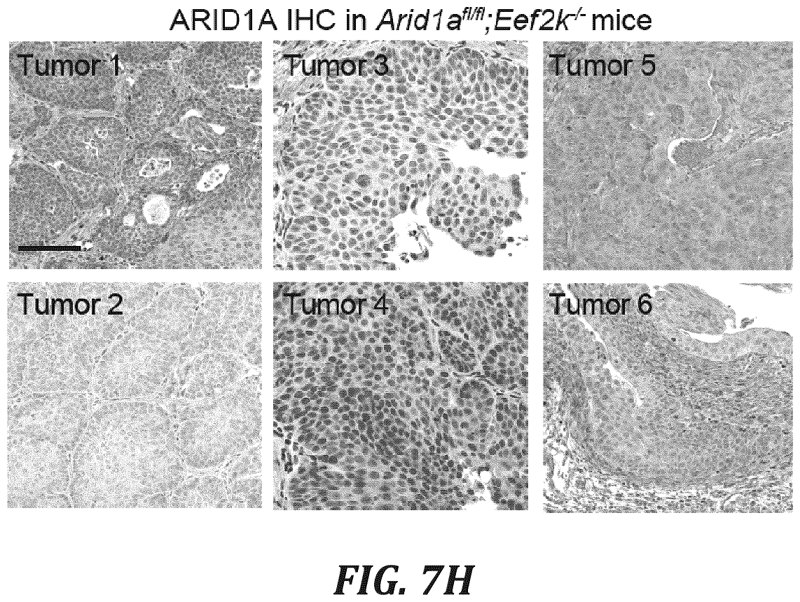

ARID1A directly interacts with Ataxia telangiectasia and RAD3-related protein (ATR) to promote double strand break end resection and ATR activation to maintain chromosomal stability in response to radiation. The data presented herein suggested that mRNA-specific translation elongation downstream of ARID1A could also promote DNA damage repair and cell survival. To determine if the decrease in translation elongation which occurred upon ARID1A loss is responsible for the impaired DDR independent of ARID1A's role as an ATR interactor, Arid1a^{fl/fl} and Arid1a^{fl/fl};Eef2k^{-/-} mice were treated with BBN for 9 days. A genetic restoration of translation elongation, was observed, which restored BRCA2, ERCC1, ERCC2, and FANCC protein abundance, and was sufficient to decrease DNA damage and apoptosis in Arid1a^{fl/fl};Eef2k^{-/-} mice (FIG. 6D and FIGS. 7A-7B). Carcinogen exposure in both mouse models was equivalent. To determine if this rescue was mediated by the catalytic activity of eEF2K, Arid1a^{fl/fl} mice were pretreated with A-484954 before a 9-day course of BBN. Like the genetic model, inhibition of eEF2K activity markedly decreased DNA damage and apoptosis (FIGS. 7C-7D).

These short-term studies raised the possibility that mRNA-specific impairments in translation elongation form a protective barrier that prevent ARID1A-deficient cells from carcinogen-mediated transformation. To directly address this question, Arid1a^{fl/fl} and Arid1a^{fl/fl};Eef2k^{-/-} mice were treated with BBN for 150 days. Arid1a^{fl/fl};Eef2k^{-/-} mice formed significantly larger bladder tumors compared to Arid1a^{fl/fl} mice (FIGS. 7E-7F). Remarkably, unlike all tumors in Arid1a^{fl/fl} mice which continued to express ARID1A protein, only 1 out of 7 Arid1a^{fl/fl};Eef2k^{-/-} tumors was positive for ARID1A (FIGS. 6K, and 7G-7H). As such, restoring translation elongation and mRNA specific translation was sufficient to drive carcinogenesis and the formation of ARID1A-deficient tumors.

### Example 8

### Mice

Arid1a^{LoxP/LoxP} (Jackson Laboratory), ROSA-LSL-Yfp^{KI/KI} (Jackson Laboratory), and UBC-Cre^{ERT2} (Jackson Laboratory). eEF2K^{-/-} animals were kindly provided by Alexey G. Ryazanov (Rutgers University). UBC-Cre^{ERT2};ROSA-LSL-Yfp^{KI/KI};Arid1a^{LoxP/LoxP} animals (mixed background) were bred to Eef2k^{-/-} animals (C57BL/6) for one generation to produce progeny heterozygous at all loci (ROSA-LSL-Yfp^{KI/+};Arid1a^{LoxP}/+; eEF2K^{+/-}) with or without UBC-Cre^{ERT2}. Heterozygous animals without UBC-Cre^{ERT2} were backcrossed once more to ROSA-LSL-Yfp^{KI/KI};Arid1a^{LoxP/LoxP} animals to reintroduce homozygosity at the ARID1A and ROSA-LSL-Yfp, while maintaining heterozygosity at the Eef2k locus (ROSA-LSL-Yfp^{KI/KI}; Arid1a^{LoxP/LoxP}; Eef2K^{+/-}; with and without UBC-Cre^{ERT2}). Of these progeny, animals with UBC-Cre^{ERT2} were bred to animals without UBC-Cre^{ERT2} to produce two parallel lineages, both in a consistent background with a single copy of UBC-Cre^{ERT2} (UBC-Cre^{ERT2}; ROSA-LSL-Yfp^{KI/KI}; Arid1a^{LoxP/LoxP}; Eef2k^{+/+} and UBC-Cre^{ERT2}; ROSA-LSL-Yfp^{KI/KI}; Arid1a^{LoxP/LoxP}; Eef2k^{-/-}). The lineages were maintained separately with in-breeding. K5-Cre^{ERT2} mice were purchased from the Jackson Laboratory. ROSA-LSL-Yfp^{KI/KI}; Arid1a^{LoxP/LoxP} animals (mixed background) were bred to K5-Cre^{ERT2} animals (congenic C57BL/6 background) for one generation to produce progeny heterozygous at all loci (K5-Cre^{ERT2/+}; ROSA-LSL-Yfp^{KI/+}; Arid1a^{LoxP/+}). These animals were backcrossed once more to ROSA-LSL-Yfp^{KI/KI}; Arid1a^{LoxP/LoxP} animals to reintroduce homozygosity at the Arid1a and ROSA-LSL-Yfp loci (K5-Cre^{ERT2/+}; ROSA-LSL-Yfp^{KI/KI}; Arid1a^{LoxP/LoxP} and K5-Cre^{+/+}; ROSA-LSL-Yfp^{KI/KI}; Arid1a^{LoxP/LoxP}) The lineage was maintained with subsequent in-breeding of these two genotypes to maintain a single copy of CreERT. NOD-SCID 7TL-2 mice were used for the preclinical trial. PDX models were obtained from Jackson Laboratory (TM01029, TM00016, TM00024). The protocols used were approved by the Fred Hutchinson Cancer Center Animal Care and Use Committee (IACUC) for all mouse studies. The number of mice used for each study is included in figure legends. All studies were conducted using 50% male and 50% female mice. All comparisons were made between litter mates. For survival studies, the body weight of the mouse was recorded and monitored every two days for the duration of the survival trial. Mice were euthanized when they exhibited >20% weight loss or met euthanasia requirements by exhibiting pain and distress.

### Example 9

### Cell Culture

Cell lines used for this study include HT1376, KU1919, HT1197, UMUC11, and Human Prostate Epithelial Cells (Human PrEC). Mouse bone marrow stromal cells were derived from WT mice. HT1197 and HT1376 cells were cultured in Eagle's Minimum Essential Medium (ATCC) supplemented with 10% fetal bovine serum (Cytiva), and 1% penicillin/streptomycin (Gibco). KU1919 cells were cultured in RPMI1640 (Gibco) supplemented with 10% fetal bovine serum, and 1% penicillin/streptomycin. UMUC11 cells were cultured in DMEM (Gibco) supplemented with 10% fetal bovine serum, and 1% penicillin/streptomycin. These cell lines were maintained in a 37° C. and 5% CO₂ incubator. All the above cell lines were generously provided by MacPherson Lab (Fred Hutchinson Cancer Center). Human Prostate Epithelial Cells (Lonza, CC-2555) were cultured using Lonza's BulletKit™ media (Lonza) according to manufacturer's instructions. Mouse bone marrow stromal cells were generated by the following method. Briefly, bone marrow cells were isolated by flushing the tibia and femur bones of WT mice with PBS. Cells were filtered through a cell strainer and cultured in Iscove's modified Dulbecco's medium (Fisher) supplemented with 15% fetal calf serum, 5% horse serum (Life Technology), 10⁻⁵ M hydrocortisone (Sigma), 1% penicillin/streptomycin, and 10⁻⁴ M β-mercaptoethanol at 33° C.

### Example 10

### Organoid Culture

Organoid lines were generated either from normal murine bladder or bladder tumors. Minced tissues were treated with 5 mg/mL collagenase type II solution (Life Technologies) diluted in Dulbecco's Modified Eagle's Medium for 1 hour at 37° C., followed by a 5-minute treatment with TrypLE (Gibco, 12604-039) treatment at 37° C. Digested tissues further dissociated using a syringe with 18G needle, and single cells were obtained after passing through a cell strainer. Cells were plated using EHS (NIH and Corning) and cultured in organoid media for 5-7 days at 37° C. Organoid base media was prepared using Advanced DMEM/F12 (Gibco) supplemented with 20% B27 (Gibco, 17504-044), 10 mM HEPES, GlutaMAX™ (Fisher), and 1.25 mM N-Acetyl-L-cysteine (Sigma). Base media was also supplemented with 50 ng/mL EGF (Peprotech), 100 ng/mL Noggin (conditioned media), 500 ng/mL R-spondin (conditioned media), 200 nM A83-01 (Tocris), and 10 μM Y-27632 (Sigma). Organoids were passaged once every week.

### Example 11

### ARID1A Re-Expression

Arid1a^{fl/fl} organoid cells were plated at a density of 10,000 cells per 15 μL EHS in 24-well ultra-low attachment plates with lentivirus expressing (dox-inducible) pLenti-puro-ARID1A and pLenti6/TR. Organoids were cultured in organoid media (see organoid culture) for 96 hours. Stable lines were generated using puromycin and blasticidin treatment.

### Example 12

### Rasgrp1 Re-Expression

Arid1a^{fl/fl} cells were transfected with Rasgrp1-pEF6 construct using Lipofectamine™ 3000. Stable lines were generated using blasticidin.

### Example 13

### Urine BCPN Analysis

Urine was collected from WT, Arid1a^{fl/fl}, and Arid1a^{fl/fl};Eef2k^{-/-} mice after 9 days of 0.075% BBN treatment. 5 μL of urine was analyzed on a Xevo QT of mass spectrometer. BCPN (N-butyl-N-(3-carboxypropyl)nitrosaime) concentrations were calculated by comparing the urine BCPN measurements to standards of known BCPN (TCI America) concentrations.

### Example 14

### Immunoblot Analysis

Cell lysates were prepared using RIPA buffer (Fisher) containing phosphatase inhibitor (Sigma) and protease inhibitor cocktail (Sigma). The lysates were incubated on ice for 30 minutes with periodic mixing by vortex and centrifuged at 13,000×g at 4° C. for 10 minutes. The supernatants were subjected to SDS-PAGE and transferred to a PDVF membrane (Bio-Rad). Membranes were blocked with 5% milk for 1 hour at room temperature. Primary antibodies were diluted in 5% milk and incubated overnight at 4° C. Blots were incubated with horse radish peroxidase- (HRP-) conjugated secondary antibodies (goat anti-rabbit IgG (Fisher) or goat anti-mouse IgG (Fisher)) in 5% milk for 1 hour at room temperature. West Pico (Fisher) was used to detect immunoreactive bands on the blot using the ChemiDoc Touch Imaging System (Bio-Rad). The following primary antibodies were used: anti-Tubulin (Sigma), anti-ARID1A (Cell Signaling Technology), anti-PTEN (Cell Signaling Technology), anti-pAKT (5473) (Cell Signaling Technology), anti-AKT1 (Santa Cruz Biotechnology), anti-p-eIF2α (S51) (Cell Signaling Technology), anti-ATF4 (Cell Signaling Technology), anti-eIF4G (Cell Signaling Technology), anti-eIF4E (Santa Cruz Biotechnology), anti-eIF4A (Cell Signaling Technology), anti-p-4EBP1 (Cell Signaling Technology), anti-4EBP1 (Cell Signaling Technology), anti-p-eEF2 (T56) (Cell Signaling Technology), anti-eEF2 (Cell Signaling Technology), anti-AURKB (Novusbio), anti-FGFR3 (Mybiosource), anti-IGF2 (Fisher), anti-KIF22 (Novusbio), anti-ODC1 (Fisher), anti-SKA1 (Novusbio), anti-BRCA2 (Abcam), anti-ERCC1 (Cell Signaling Technology), anti-ERCC2 (Cell Signaling Technology), anti-FANCC (Abcam), anti-p-AMPK (Thr172) (Cell Signaling Technology), anti-AMPK (Cell Signaling Technology), anti-H3K27me3 (Cell Signaling Technology), anti-H3 (Cell Signaling Technology), anti-puromycin (EMD Millipore), anti-Rasgrp1 (Origene), anti-p70RSK (Cell Signaling Technology), anti-p-p70RSK (T389) (Cell Signaling Technology), anti-eEF2K (Abcam), anti-p-eEF2K (S398) (ECM Bio), anti-p-eEF2K (S366) (Abcam), anti-ARID1A (Santa Cruz Biotechnology), and anti-ARID1A (Sigma).

### Example 15

### Mouse Genotyping

Mouse tissue was incubated in 1.67 mg/mL of Proteinase K solution (Sigma) overnight at 55° C., then subsequently extracted using Phenol: chloroform:isoamyl alcohol (Ambion) followed by ethanol precipitation. Genotypes were confirmed using PCR with GoTaq® Green Master Mix (Promega) followed by agarose gel electrophoresis (Bio-Rad). Primers used for genotyping PCR, ARID1A (forward, 5′-GTAATGGGAAAG CGACTACTGGAG-3′ (SEQ ID NO: 1); reverse, 5′-TGTTCGTTTTTGTGGCGGGAG-3′; SEQ ID NO:2)), UBC-Cre^{ERT} (forward, 5′-GCGGTCTGGCAGTAAAAACTATC-3′ (SEQ ID NO:3); reverse, 5′-GTGAAACAG CATTGCTGTCACTT-3′ (SEQ ID NO:4)); (internal control: forward, 5′-CTAGGCCACAGAATTGAAAGATCT-3′ (SEQ ID NO:5); reverse, 5′-GTAGGTGGAAATTCTAGCATCATCC-3′; SEQ ID NO:6)); ROSA-LSL-Yfp^{KI/KI} (WT: forward, 5′-CTGGCTTCTGAGGACCG-3′ (SEQ ID NO:7); reverse, 5′-CAG GACAACGCCCACACA-3′ (SEQ ID NO:8)). YFP (forward, 5′-AGGGCGAGGAGC TGTTCA-3′ (SEQ ID NO:9); reverse, 5′-TGAAGTCGATGCCCTTCAG-3′ (SEQ ID NO:10)), K5-Cre^{ERT2} (WT: forward, 5′-GCAAGACCCTGGTCCTCAC-3′ (SEQ ID NO:11); reverse, 5′-GGAGGAAGTCAGAACCAGGAC-3′ (SEQ ID NO:12)). CreERT (forward, 5′-GCAAGACCCTGGTCCTCAC-3′ (SEQ ID NO:13); reverse, 5′-ACCGGC CTTATTCCAAGC-3′ (SEQ ID NO:14)), Eef2k^{-/-} (WT: forward, 5′-GGCCGGC TGCTAGAGAGTGTC-3′ (SEQ ID NO:15); reverse, 5′-CATCAGCTGATTGTAGTGG ACATC-3′ (SEQ ID NO:16)). eEF2K KO (forward, 5′-TGCGAGGCCAGAGGCCAC TTGTGTAGC-3′ (SEQ ID NO:17); reverse, 5′-CAGGGCCTGCTTTCTTGGTGGCAG-3′ (SEQ ID NO:18)).

### Example 16

### Hematoxylin and Eosin Staining (H&E), Immunohistochemistry (IHC), Immunofluorescence (IF)

Bladder samples were collected, fixed in 4% paraformaldehyde (PFA), dehydrated with ethanol, and embedded in paraffin wax. Paraffin blocks were sectioned (5 μm) using a rotary microtome and hematoxylin and eosin (H&E) stained. Immunohistochemistry (IHC) staining was performed after rehydrating the tissue. In brief, antigen retrieval was performed by preheating in sodium citrate buffer (pH 6.0 or pH 9) solution for 7-30 minutes at 95° C.-125° C. in a pressure cooker. Hydrogen peroxidase (Vector Laboratories) treatment was done for 5 minutes to quench endogenous peroxides. Tissue was blocked in 1% BSA for 1 hour. Primary antibodies were applied and incubated at room temperature for 1 hour in a humidified chamber followed by secondary antibody (EnVision™ system HRP-labeled polymer, Dako, K4003). Color development was achieved by applying 3,3′-diaminobenzidine (DAB) solution (Agilent Technology) for 2 to 5 minutes, depending on the primary antibody. The sections were counterstained with hematoxylin (Agilent Technology), washed, and cover-slipped using aqueous-based mounting medium (Agilent Technology). All IHC slides were scanned using Aperio ScanScope AT Turbo (Leica Biosystems). Following primary antibodies were used for this study: anti-ARID1A (Sigma), anti-eEF2 (Abcam), anti-p-eEF2 (T56) (Abcam), eEF2K (Aviva Systems Biology), ERK1/2 (Cell Signaling Technology), p-ERK1/2 (Cell Signaling Technology), MEK1/2 (Cell Signaling Technology), p-MEK1/2 (Cell Signaling Technology), p90RSK (Abcam), p-p90RSK (Cell Signaling Technology), RASGRP1 (Fisher), Ki67 (Cell Signaling Technology), γH2AX (Cell Signaling Technology), CC3 (Cell Signaling Technology).

Immunofluorescent (IF) staining was performed in the following manner. Antigen retrieval was conducted by preheating in sodium citrate buffer (pH 6.0) for 30 minutes at 95° C. in a pressure cooker. Tissue was blocked in 1% BSA solution for 2 hours or in M.O.M® blocking reagent (Vector Laboratories) for 1 hour at room temperature, followed by blocking in 1% BSA solution for 2 hours. Primary antibodies were applied overnight at 4° C. in a humidified chamber, and secondary antibodies (Alexa Fluor® 594; Alexa Fluor® 488; or Alexa Fluor® 647 (Invitrogen)) were applied for 1 hour at room temperature. All IF slides were mounted using ProLong™ gold mounting media with DAPI (Fisher Scientific, P36935). IF slides were scanned using an Aperio Scanscope FL (Leica Biosystems). The following primary antibodies were used for IF study: anti-Cytokeratin 5 (Biolegend Inc), anti-puromycin (EMD Millipore), anti-p-eEF2K (S366) (Cell Signaling Technology), anti-ARID1A (Sigma), or anti-Cytokeratin 5 (Abcam). Image analysis was done using semiautomated image analysis software (HALO, Indica Labs).

### Example 17

ChIP qPCR

5×10⁶ cells were used per immunoprecipitation reaction using the SimpleChIP® Enzymatic Chromatin IP Kit (Cell Signaling Technology) as per manufacturers' instruction. Chromatin was cross-linked with 1% formaldehyde in PBS for 10 minutes at room temperature and quenched by the addition of 10× glycine and incubated for 5 minutes at room temperature, followed by a wash with PBS. Crosslinked chromatin was fractionated by digestion with micrococcal nuclease (0.2 μL per IP) for 20 minutes at 37° C. This was followed by 6 cycles of sonication (20 seconds on, 30 seconds off, 50% amplitude) using a cup horn sonicator (Qsonica Q500). IPs were performed using 1:100 anti-ARID1A (D2A8U) (Cell Signaling Technology) and equivalent concentration of rabbit anti-IgG control (Cell Signaling Technology) at 4° C. overnight. Crosslinks were reversed with Proteinase K at 65° C. for 2 hours and DNA was purified using the SimpleChIP® Enyzmatic Chromatin IP Kit. Chromatin immunoprecipitation quantitative real-time PCR (ChIP-qPCR) was performed as per manufacturers' instructions using Bio-Rad CFX384 Real-Time System using SsoAdvanced™ Universal SYBR® Green Supermix (Bio-Rad). Primer sequences against ARID1A target regions used for ChIP-qPCR (forward, 5′-CTTCGAATCCTGCCCCCATT-3′ (SEQ ID NO:19); reverse, 5′-TCT TGGGCTGGGAGAGATGA-3′ (SEQ ID NO:20)).

### Example 18

### In Vivo and In Vitro Puromycin Incorporation Assay

For the in vivo puromycin incorporation assay, mice were injected i.p. with 200 μL of 2.5 mM puromycin (Fisher) and euthanized after 1 hour. For in vitro puromycin incorporation assay, cells were treated with 1 μM puromycin for 30 minutes at 37° C.

### Example 19

### RNA Sequencing and Polysome-Bound RNA Sequencing

Organoids were cultured for 96 hours before harvesting for RNA extraction or polysome profiling (see polysome profiling). Polysome fractions were mixed 1:1 with TRIzol™ Reagent (Invitrogen) and stored at −8.0° C. until processing. The sub-polysome and polysome fractions were individually pooled and processed for sequencing. RNA was extracted from each pool using Direct-zol™ Miniprep Plus kits (Zymo Research Corp.) and concentrations were assessed by Qubit. ERCC RNA Spike-In Control Mix (Fisher) was serial diluted (1:1000) and 2 μL was used for every 100 ng of RNA. RNA-seq libraries were constructed using TruSeq® Stranded mRNA Library Prep Kit (Illumina) with the IDT for Illumina (TruSeq® RNA UD Indexes: Illumina) following manufacturer's instructions. The sequencing was done on the Illumina NovaSeq 6000 using the SP-100 flow cell sequencing kit (Illumina), 50PE run configuration.

### Example 20

### In Vitro [³⁵S]-Methionine Labeling Assay

Cells were plated at a density of 10,000 cells per 50 μL EHS (NIH) and cultured with organoid media (see organoid culture). Cells were treated with 0.05 mCi/mL [³⁵S]-methionine (Perkin Elmer) at 37° C. for 1 hour. Total cell lysates were prepared and subjected to immunoblot analysis. X-ray film was used to capture the radioactive signal. To check de novo protein synthesis in ARID1A re-expressing organoids, cells were treated with Doxycycline (0.1 μg/mL) for 48 h before performing [³⁵S]-methionine labeling assay.

### Example 21

### Cell Viability Assay

WT bladder tumor organoids were established using following methods. Briefly, WT bladder tumor tissue chunks for Arid1a non-recombined Arid1a^{fl/fl} mice were incubated in 5 mg/mL collagenase type II (Life Technologies) diluted in Dulbecco's Modified Eagle's Medium for 1 hour at 37° C., followed by a 5-minute digestion with TrypLE (Gibco). Digested tissues were dissociated using a syringe with 18G needle, and single cells were obtained after passing through a cell strainer. Cells were plated at a density of 20,000 cells per 50 μL of EHS (NIH) on 24-well ultra-low attachment plates (Corning) and cultured in organoid media (see organoid culture) to establish WT bladder tumor organoids. Arid1a^{fl/fl} bladder tumor organoids were generated from the WT parental line using lentivirus expressing Cre-recombinase (Addgene). Both WT and Arid1a^{fl/fl} tumor organoids were isolated by fluorescent activated (YFP) cell sorting and propagated as single-cell clones. To measure the differential cytotoxicity of homoharringtonine (HHT), the CellTiter-Glo® 2.0 Assay (Promega) was used. Two thousand WT and Arid1a^{fl/fl} tumor cells were embedded in 5 μL of Matrigel® and plated on a 96-well plate in organoid media. One day post seeding, media was replaced with media containing either 100 nM or 1 μM of homoharringtonine (HHT). After a 48-hour HHT treatment, the CellTiter-Glo® 2.0 Assay was performed according to manufacturer's instructions. HT1376, KU1919, HT1197, and UMUC11 cells (5000 cells/well) were plated on a 96-well plate. Cells were treated with various concentration of HHT (1, and 10 μM) at 48 hours after plating. After 24 hours of HHT treatment, the CellTiter-Glo® 2.0 Assay was performed according to manufacturer's instructions.

### Example 22

siRNA Knockdown

siARID1A (ON-TARGETplus SMARTpool siRNA; Dharmacon) were transfected into human PrEC cells according to manufacturer's protocol. Briefly, human PrEC cells were plated in 6-well plates and siRNAs were transfected using Lipofectamine™ 2000 transfection reagents (Fisher). Cells were harvested 72 hours after transfection and subjected to immunoblot to verify knockdown.

### Example 23

### Polysome Profiling

WT or Arid1a^{fl/fl} cells were plated at a density of 10,000 cells per 50 μL EHS (NIH) and cultured with organoid media for 96 hours (see organoid culture). Cells were treated with 100 μg/mL (final concentration) cycloheximide (Sigma) for 10 minutes at 37° C. Cells were extracted from EHS by incubating in cell recovery solution (Corning) for 1 hour at 4° C. Cell pellets were lysed on ice in 350 μL of polysome lysis buffer (10 mM Tris-HCl pH 7.4; Ambion: AM9851 (pH 7.0) and AM9856 (pH 8.0)), 132 mM NaCl (Ambion), 1.4 mM MgCl₂ (Ambion), 19 mM DTT (Sigma), 142 μg/mL cycloheximide (Sigma), 0.1% Triton X-100 (Fisher), 0.2% NP-40 (Pierce), 607 U/mL SUPERase-In™ RNase Inhibitor (Life Technologies) with periodic vortex mixing. Lysates were clarified by centrifugation at 9300×g for 5 minutes and supernatants were transferred to fresh tubes. Protein quantification was performed on each lysate by Bradford assay (Bio-Rad) and analyzed using a BioTek Epoch™ Microplate Spectrophotometer. A portion of each lysate was saved as an input total RNA sample. For each lysate, 850 μg protein in 265 μL of the supernatant was layered onto 10% to 50% (w/v) linear sucrose gradients (Fisher) containing 2 mM DTT (Sigma) and 100 μg/mL heparin (Sigma). The gradients were centrifuged at 37,000 rpm for 2.5 hours at 4° C. in a Beckman SW41Ti rotor in Seton 7030 ultracentrifuge tubes. After centrifugation, samples were fractionated using a Biocomp Gradient Station (Biocomp) by upward displacement into collection tubes, through a Bio-Rad EM-1 UV monitor (Bio-Rad) for continuous measurement of the absorbance at 254 nm.

### Example 24

### Ribosome Half-Transit Time

Cells were plated at a density of 10,000 cells per 50 μL EHS in 24-well ultra-low attachment plates and cultured with organoid media (see organoid culture) for 96 hours. Cells were treated with 10 μCi/mL [³⁵S]-methionine (Perkin Elmer) and incubated at 37° C. At indicated time points (5, 10 and 15 minutes) after labeling, cells were harvested in ice-cold PBS containing 100 μg/mL cycloheximide. Cells were extracted from EHS using TrypLE treatment and resuspended in 250 μL of RBS buffer (10 mM NaCl, 10 mM Tris-HCl at pH 7.4, 15 mM MgCl₂, 100 μg/mL heparin and protease inhibitor). 35 μL of lysis buffer (10% Triton X-100, 10% deoxycholate) was also added to lyse the cells. Cell suspension was incubated on ice for 10 minutes with periodic mixing by vortex. Nuclei and mitochondria were separated by centrifugation for 5 minutes at 10,000×g at 4° C. Supernatant (PMS) was removed and equal volume of polysome buffer (25 mM NaCl, 10 mM MgCl₂, 25 mM Tris-HCl at pH 7.4, 0.05% Triton X-100, 0.14 M sucrose, 500 μg/mL heparin) was added. Polysomes were pelleted by centrifugation of half of the volume at 87,000 rpm for 2 hours using the TLA100 rotor and the supernatant (PRS) was removed. PMS represents nascent and completed proteins while PRS represents only completed proteins. Equal volume of PMS and PRS samples were precipitated on glass microfiber filters (Sigma) using 20% TCA for 20 minutes on ice. Filters were washed with 10% TCA twice and once with ice-cold ethanol. Filters were air dried before taking counts. Liquid scintillation cocktail (Fisher) was added prior to liquid scintillation counting.

### Example 25

### TMT Mass Spectrometry

Disulfide bond reduction/alkylation: Protein solutions (120 μg) were diluted to 2.4 μg/L in 50 mM HEPES pH 8.5. Disulfide bonds within the proteins were reduced by adding tris (2-carboxyethyl)phosphine to a concentration of 5 mM and mixing at room temperature for 15 minutes. The reduced proteins were alkylated by adding 10 mM 2-chloroacetamide to a concentration of 10 mM and mixed in the dark at room temperature for 30 minutes. Excess 2-chloroacetamide was quenched by adding dithiothreitol to a concentration of 10 mM and mixing at room temperature for 15 minutes.

Protein precipitation and protease digestion: Samples (120 μg) were diluted to 1 μg/L with 100 mM ammonium bicarbonate in a 1.5 mL Eppendorf® low-bind tube. Protein precipitation was carried out as follows: 400 μL of methanol was added to the sample and mixing by vortex for 5 seconds, 100 μL of chloroform was added to the sample and mixing by vortex for 5 seconds, 300 μL of water was added to the sample and mixing by vortex for 5 seconds, the samples were centrifuged for 1 minute at 14,000×g, and the aqueous and organic phases were removed, leaving a protein wafer in the tube. The protein wafers were washed with 400 μL of methanol and centrifuged at 21,000×g at room temperature for 2 minutes. The supernatants were removed, and the pellets were allowed to air dry. The samples were resuspended in 70 μL 100 mM HEPES (pH 8.5) and digested with rLys-C protease (100:1, protein to protease ratio) with mixing at 37° C. for 4 hours. Trypsin protease (100:1, protein to protease ratio) was added and the reaction was mixed overnight at 37° C.

TMTpro18plex labeling: Each TMTpro18plex labeling reagent (Fisher, A44521 and A52048) was brought up in 30 μL acetonitrile and added to their assigned digested peptide solution (100 μg), yielding a final organic concentration of 30% (v/v), and mixed at room temperature for 1 hour. A 2-ag aliquot from each sample was combined, dried to remove the acetonitrile, processed with a Zip-Tip™ pipette tip with C18 resin, and analyzed via LC/MS as a “label check”. The label check was used to check that the labeling efficiency of the TMT reagent exceeded 97% and to determine the volumes from each sample to be used for equalizing the protein amounts when combining the samples. After the label check, the reactions were quenched with hydroxylamine to a final concentration of 0.3% (v/v) and mixing, for 15 minutes. The TMTpro18plex labeled samples were pooled at 1:1 ratio based on the equalization volumes determined in the label check and concentrated in a Speedvac™ to remove acetonitrile. Half of the material was desalted with an Oasis HLB 3 cc cartridge and dried with a Speedvac™.

bRP Fractionation: The desalted TMT sample was resuspended in 100 μL of 10 mM ammonium bicarbonate pH 8. The material was loaded on to a Zorbax® 2.1 cm×150 mm (5-μm particle size) Extend-C18 column connected to a HPLC equipped with a diode array detector and fraction collector. The sample was gradient-eluted from the column at a flowrate of 250 μL/min over 55 minutes using a combination of solvents “A” (10 mM ammonium bicarbonate) and “B” (acetonitrile). The elution profile used was as follows: from 0 to 5 minutes “B” was held at 1%, from 5 to 55 minutes “B” varied from 5% to 40% followed by an increase to 90% “B” over 5 minutes, and then a hold for an additional 5 minutes at 90% “B”. The UV signal was monitored at 210 nm and fractions were collected every 50 seconds, resulting in 96 fractions. The fractions were combined into 24 pools by concatenating every 24^{th} fraction into a pool (pool 1=fractions 1, 25, 49, and 73, pool 2=fractions 2, 26, 50, and 74, and the like). The pools were taken to dryness by vacuum centrifugation and subsequently analyzed by LC/MS.

LC/MS: The dried basic reverse phase fractions were brought up in 2% acetonitrile in 0.1% formic acid (20 μL) and analyzed (2.5 μL) by LC/ESI MS/MS with a Easy-nLC™ 1200 coupled to a tribrid Orbitrap Eclipse™ with FAIMS pro mass spectrometer. In-line de-salting was accomplished using a reversed-phase trap column (100 μm×20 mm) packed with Magic CisAQ (5-μm, 200 Å resin) followed by peptide separations on a reversed-phase column (75 μm×270 mm) packed with ReproSil®-Pur CisAQ (3-μm, 120 Å resin) directly mounted on the electrospray ion source. A 120-minute gradient from 4% to 44% B (80% acetonitrile in 0.1% formic acid/water) at a flow rate of 300 nL/min was used for chromatographic separations. The temperature of the chromatographic column was maintained at 40° C. using a heating blanket. A spray voltage of 2300 V was applied to the electrospray tip in-line with a FAIMS pro source using varied compensation voltages of −40 V, −60 V, −80 V while the Orbitrap Eclipse™ instrument was operated in the data-dependent mode. MS survey scans were in the Orbitrap (Normalized AGC target value 300%, resolution 120,000, and max injection time 50 ms) using a 3 s cycle time and MS/MS spectra acquisition were also detected in the Orbitrap (Normalized AGC target value of 250%, resolution 50,000 and max injection time 100 ms) using higher energy collision-induced dissociation (HCD) activation with HCD collision energy of 38%.

### Example 26

### Preclinical Trials

Tumor pieces (1×1×1 mm) were implanted into the flank of 4-6 weeks old NOD-SCID γ IL-2 mice. Xenografts were measured every 2 days and tumor volume was calculated using the formula (L×W×W)/2, where L is the length of the tumor and W its width. When tumors reached a total volume of 100 mm³, animals were randomly selected for one of two treatment arms: Homoharringtonine (Carbosynth) at 0.7 mg/kg dissolved in PBS or vehicle (PBS) twice daily by oral gavage.

### Example 27

### CUT&Tag Chromatin Profiling

CUT&Tag was performed following the published protocol, with some modifications. Briefly, cells were harvested and prepared into homogenous suspension in PBS, aliquoted with 10% DMSO and slow-frozen to −80° C. in isopropanol freezing chambers. CUT&Tag was performed with native nuclei. Cells were thawed at 37° C. for 1 minute, washed twice with PBS and centrifugation at 600×g for 3 minutes, and resuspended into ice-cold NE1 buffer (20 mM K-HEPES pH 7.9, 10 mM KCl, 10% Triton X-100, 20% Glycerol, 0.5 mM spermidine, and EDTA-free protease inhibitor). Cells were kept on ice for 10 minutes, centrifuged at 1400×g for 3 minutes using a swinging-bucket rotor, pellets were resuspended in Wash buffer (20 mM Na-HEPES pH 7.5, 150 mM NaCl, 0.5 mM spermidine, and EDTA-free protease inhibitor), nuclei were counted using Vi-CELL© Cell Viability Analyzer (Beckman Coulter) and aliquoted into 0.6 mL low-binding flip-cap tubes for CUT&Tag experiments.

500,000 nuclei were used for each CUT&Tag experiment targeting a histone posttranslational modification, while 200,000 nuclei were used for ARID1A. Bio-Mag®Plus Concanavalin A coated magnetic beads (Bangs Laboratories) were equilibrated with binding buffer (20 mM K-HEPES pH7.9, 10 mM KCl, and 1 mM each CaCl₂ and MnCl₂). To each sample, 10 μL of activated beads were added and held at room temperature for 5 minutes with occasional gentle mixing. Beads (with bound nuclei) were magnetized, supernatants were removed, beads were washed once with 400 μL antibody buffer (Wash buffer supplemented with 2 mM EDTA and 0.05% Digitonin) and resuspended in 200 μL antibody buffer containing the respective primary antibody in 1:100 v/v dilution (H3K27me3-Cell Signaling Technology—Millipore-Sigma; ARID1A—Cell Signaling Technology)). Primary antibody incubations were performed on a rotating platform overnight at 4° C. Beads were magnetized, supernatant removed, washed once with 400 μL Dig-Wash (Wash buffer supplemented with 0.05% Digitonin), resuspended in 200 μL Dig-Wash containing guinea pig α-rabbit IgG secondary antibody (Antibodies-online.com) at 1:100 dilution. Secondary antibody incubations were performed on a rotating platform for 30 minutes at room temperature. Beads were magnetized, supernatant removed, washed twice with 400 μL Dig-Wash to remove unbound antibodies, and resuspended in 200 μL Dig-Med buffer (Dig-Wash buffer, except containing 300 mM NaCl) with 1:200 dilution (˜0.04 M) of proteinA-Tn5 transposase fusion protein (pA-Tn5) pre-loaded with double-stranded adapters with 19-mer mosaic ends. pA-Tn5 incubations were performed on a rotating platform for 1 hour at room temperature. Beads were magnetized, supernatant removed, washed three times with 400 μL Dig-Med to remove unbound pA-Tn5, and resuspended in 300 μL Tagmentation buffer (Dig-Med supplemented with 10 mM MgCl₂). Tagmentation reactions were performed by incubating samples at 37° C. on a rotating platform for 1 hour. Tagmentation reactions were stopped by sequentially adding 10 μL of 0.5 mM EDTA, 3.1 μL of 10% SDS (1% final), and 2 μL of 20 mg/mL Proteinase K, mixed well, and incubated in 50° C. water bath for 1 hour. DNA was extracted using phenol-chloroform-isoamyl alcohol (25:24:1), re-extracted with one volume CHCl₃, aqueous phase (˜300 μL) was transferred to fresh tube, and DNA was precipitated by adding 900 μL ethanol overnight at −20° C. followed by centrifugation for 45 minutes at 13,000 rpm and 4° C. Pellets were rinsed with 100% ice-cold ethanol, air-dried, dissolved in 30 μL 0.1×TE (1 mM Tris pH 8, 0.1 mM EDTA) supplemented with RNase A (1:400 dilution of 10 mg/mL), and incubated in 37° C. water bath for 15 minutes.

To amplify libraries, 21 μL DNA was mixed with 2 μL each of a universal i5 primer and a uniquely barcoded i7 primer, using a different barcode for each sample, and 25 μL of NEB Next HiFi 2×PCR Master mix. Samples were placed in a thermocycler with heated lid using the following cycling conditions: 72° C. for 5 minutes (gap filling); 98° C. for 30 seconds (denaturation); 13 cycles of 98° C. for 10 seconds and 63° C. for 10 seconds (combined annealing and extension); final extension at 72° C. for 1 minute and held at 8° C. Post-PCR clean-up was performed using a double-sided clean-up method. 25 μL (0.5 X) of AMPure XP beads (Beckman Counter) was mixed with the libraries and incubated for 10 minutes at room temperature to remove large DNA fragments (>1300 bp). Beads were magnetized, supernatants were removed to fresh tubes and mixed with 45 μL (1.4 X−0.5 X=0.9 X) of AMPure XP beads and incubated for 10 minutes at room temperature. Beads were magnetized and the supernatants containing unused PCR primers were discarded. Beads were washed three times with 80% ethanol and eluted in 30 μL 10 mM Tris pH 8.0. Libraries were sequenced for 25 cycles in 25 bp paired-end mode on the Illumina HiSeq 2500 sequencing platform or 50 bp paired-end mode on a sequencing system (Illumina NextSeq 2000).

### Example 28

### Flow Cytometry

To isolate basal urothelial cells, bladder tissue was digested with collagenase II and generated single cell suspension (for details see organoid culture). Fluorescence-conjugated antibodies were then added to cell suspensions and stained for 30 minutes on ice. Live basal cells were separated by flow cytometry after staining with 4′,6-diamidino-2-phenylindole (DAPI), allophycocyanin labeled epithelial cell adhesion molecule (EPCAM-APC; an epithelial marker) and phycoerythrin labeled CD49f (CD49f-PE; a basal cell marker). 0.125 μg of EPCAM-APC (Invitrogen) and CD49f-PE (Fisher) were used respectively per test containing 1×10⁸ cells.

### Example 29

### Cell Cycle Analysis

Cells were fixed using pre-cooled 66% ethanol at 4° C. for at 2 hours. Propidium iodide staining was performed following manufacturer's instruction (Abcam). The cells were read using flow cytometry (Fortessa X50) and cell cycle phases were analyzed using FlowJo software.

### Example 30

### Comet Assay

The alkaline comet assay was performed using CometAssay® kit (Trevigen) according to manufacturer's instructions. Briefly, organoids were treated with BCPN ((N-butyl-N-(3-carboxypropyl)nitrosamine); 100 μg/mL) for 96 hours. Cells were extracted from EHS using TrypLE and resuspended in PBS (1×10⁴ cells/mL). The adjusted volume of cells was mixed with LMA (Low melting agarose) and plated onto comet slides. Comet slides were kept at 4° C. for 30 minutes followed by overnight incubation in lysis buffer. The following day, comet slides were washed with water and immersed in alkaline unwinding solution (200 mM NaOH, 1 mM EDTA, pH >13) at 4° C. for 1 hour in the dark. Slides were removed and placed into a horizontal electrophoresis tank containing alkaline electrophoresis buffer (200 mM NaOH, 1 mM EDTA, pH >13). Electrophoresis was performed at 21 V (300 mA) for 30 minutes. Slides were washed twice in water and kept in 70% ethanol for 5 minutes. SYBR™ Gold dye was used to stain the DNA and comet slides were scanned using Zeiss Axio Imager Z2 microscope (TissueGnostics, Austria). Damaged DNA molecules migrate slower than undamaged DNA leaving a characteristic appearance of a comet. Comet heads represent undamaged DNA and length of the comet tails represent severity of damaged DNA.

### Example 31

### Clonogenic Assay

Cells were plated in 24-well plates at a density of 300 cells/well using 50% Matrigel® for 24 hours, prior to the addition of 20 mM difluoromethylornithine (DFMO) (Tocris Bioscience) and 10 μg/mL FGFR3 antibody (Creative bio labs). DFMO containing media was removed and replaced every 48 hours. Colonies were counted after 10 days using Evos® FL cell imaging system.

### Example 32

### Organoid Embedding and Staining

Organoids were fixed using 4% PFA for 2 hours at room temperature on a shaker followed by a 30-minute incubation on ice to achieve complete removal of EHS. Intact organoids were collected as a pellet after centrifugation at 1400 rpm for 5 min and washed with ddH₂O. The pellet was further washed with 0.2% BSA and organoids were mixed with HistoGel™ (Epredia). The organoid and HistoGel™ suspension was centrifuged at 1400 rpm for 5 minutes and kept on ice for 30 minutes. The solidified HistoGel™ structure was stored at 4° C. until ready for processing and paraffin embedding. Organoid sections (5 m) were deparaffinized in CitriSolv™ (Fisher, 4355121) for 10 minutes and rehydrated using 100% (2×), 90% (2×) and 70% (2×) ethanol for 5 minutes each. Antigen retrieval was performed by preheating in sodium citrate buffer (pH 6.0) solution for 10 minutes at 121° C. in a pressure cooker. Organoid sections were blocked in 1% BSA solution for 1 hour followed by overnight incubation in primary antibody (anti-RASGRP1: Fisher) at 4° C. in a humidified chamber. Secondary antibody (Alexa Fluor® 594: Invitrogen) incubation was performed for 1 hour at room temperature. All slides were mounted using ProLong™ gold mounting media with DAPI (Fisher Scientific, P36935). Slides were scanned using an Aperio Scanscope FL (Leica Biosystems) and image analysis was done using semiautomated image analysis software (HALO, Indica Labs).

### Example 33

### Drug Administration

Tamoxifen: For all in vivo experiments, mice were given a total of 30 mg of tamoxifen (Sigma) divided in 6 doses by oral gavage over the span of 3 weeks (2 doses/week). Tamoxifen was prepared in corn oil. BBN (Fisher) was administrated (0.075%) in drinking water ad libitum for days as required for specific experiment (see experiment schema in figures). eEF2K inhibitor (A-484954): For all eEF2K inhibitor experiments, mice received A-484954 (Sigma) daily (10 mg/kg body weight; IP) for 15 days.

### Example 34

### RNA-Sequencing and Polysome Sequencing Analysis

Raw sequencing reads were checked for quality using FastQC (See Babraham Bioinformatics website). RNA-seq reads were aligned to the UCSC mm10 assembly using STAR2 and counted for gene associations against the UCSC genes database with HTSeq. The normalized count data was used for subsequent Principal component analysis and Multidimensional scaling (MDS) in R. Differential Expression analysis for RNA-Seq data was performed using R/Bioconductor package edgeR. A log 2 fold change cutoff of 0.33 (fold change of 25%) and FDR <0.05 was used to find transcriptionally regulated genes. Genome wide polysome to sub-polysome ratio analysis was performed using xtail. Heatmaps were made using R/Bioconductor package pheatmap (website of the Comprehensive R Archive Network; pheatmap). Volcano plots were made using ggplot2 in R. For the ERCC Spike-In analysis, Bowtie2 was used for aligning the polysome sequencing data to 92 sequences from ERCC92 sequences. HTSeq-count was further used to the number of reads for each of the 92 sequences. The counts were normalized using voom. Log₂ normalized counts were used to make scatter plots in R.

### Example 35

### CUT&Tag Data Analysis

Paired-end Mus musculus reads were mapped to UCSC mm10 using Bowtie2 version 2.2.2 with parameters --end-to-end --very-sensitive --no-mixed --no-discordant -q --phred33 -I 10 -X 700. Continuous-valued data tracks (bedGraph and bigWig) were generated using genomecov in bedtools v2.30.0 (-bg option) and normalized as fraction of total counts. Genomic tracks were displayed using Integrated Genome Browser (IGB). Heatmaps and average plots were generated using computeMatrix and plotHeatmap operations in deepTools v3.5.1. Scores were averaged over 50 bp non-overlapping bins with the transcription start sites (TSS) of genes as reference points and plotted as the mean. Box-and-whiskers plots were generated with GraphPad Prism. Average scores were computed for 2 kb bins with TSS coordinates as the midpoint using the multiBigwig Summary operation in deepTools v3.5.1. Outliers (identified using the ROUT method, Q=1%) were discarded. Boxes show the 75^{th} and 25^{th} percentiles and the median. The Tukey method was used to plot whiskers. CUT&Tag data were analyzed as described (See the protocols.io processing-and-analysis-tutorial web site). Briefly, adapters were clipped and paired-end Mus musculus reads were mapped to UCSC mm10 using Bowtie2 with parameters: --very-sensitive-local --soft-clipped-unmapped-tlen --dovetail --no-mixed --no-discordant -q --phred33 -I 10 -X 1000. Spike-in E. coli reads were mapped to Ensembl masked R64-1-1 with parameters: --end-to-end --very-sensitive --no-overlap --no-dovetail --no-unal --no-mixed --no-discordant -q --phred33 -110 -X 700.

### Example 36

### TMT Mass Spectrometry Data Analysis

Protein database searching and quantification of TMT reporter ions was performed using Proteome Discoverer 2.5 (Thermo Scientific, San Jose, CA). The data were searched against a Mouse database (UP00000598 Human 030721) that included common contaminants (cRAPome). Searches were performed with settings for the proteolytic enzyme trypsin. Maximum missed cleavages were set to 2. The precursor ion tolerance was set to 10 ppm and the fragment ion tolerance was set to 0.6 Da. Dynamic peptide modifications were set for oxidation on methionine (+15.995 Da) and modifications on the protein N-terminus, consisting of acetylation (+42.011 Da), Met-loss (−131.040 Da), and Met-loss+Acetyl (−89.030 Da). Static modifications were set for TMTpro on any N-terminus (+304.207 Da), TMTpro on lysine (+304.207 Da), and carbamidomethyl on cysteine (+57.021 Da). Sequest HT was used for protein database searching and Percolator was used for peptide validation.

Peptide to spectrum matches (PSMs) were filtered to a 1% false discovery rate and the resulting proteins were further filtered to a 1% false discovery rate. TMT channels were normalized to the largest channel intensity. Quantitative values were transformed to log 2 values and missing data were imputed by 50% of global minimum intensity. To delineate differentially expressed proteins, both pairwise two-sample t-tests and Wilcoxon rank-sum tests were conducted (R package). The Benjamini-Hochberg procedure was used for controlling familywise error rate (FWER).

### Example 37

### Ribosome Half-Transit Time Data Analysis

[³⁵S]-methionine incorporation in nascent and completed protein within the PMS and completed protein within PRS was deduced by linear regression analysis. Ribosome half-transit time was calculated by the displacement of regression lines (PMS and PRS) at time 300 seconds.

### Example 38

### % GC Content in Coding Sequence Analysis

To compare GC content of different classes of open reading frames (ORFs), Mus musculus UCSC genome sequence mm39 and Ensembl transcript annotations GRCm39 v106 were downloaded. Unique transcripts were identified for each protein coding gene by first checking for consensus coding sequence annotation (CCDSID) and then choosing the transcript with the highest support level. GC content of each CCDS ORF was calculated using letterFrequency function from the Biostrings R package. GC content for ORF groups that were transcriptionally up-regulated and those with high P/S ratio were plotted in comparison to the remaining ORFs using the geom_violin function from the ggplot2 R package. Statistical significance of differences in GC content between different ORF groups were calculated using the paired Wilcoxon test.

1. A method for treating a SWI/SNF chromatin remodeling complex mutant tumor or cancer in a subject in need thereof, the method comprising:
administering to the subject a therapeutically effective amount of an agent effective in preventing, inhibiting, or suppressing mRNA translation elongation.

2. The method of claim 1, wherein the SWI/SNF chromatin remodeling complex mutant tumor or cancer comprises an ARID1A-deficient tumor or cancer.

3. The method of claim 1, wherein the agent effective in preventing, inhibiting, or suppressing mRNA translation elongation comprises an agent effective in inhibiting or suppressing the expression or activity of eukaryotic elongation factor 2 (eEF2).

4. The method of claim 1, wherein the agent effective in preventing, inhibiting, or suppressing mRNA translation elongation inhibits or suppresses the activity of eukaryotic elongation factor 2 (eEF2).

5. The method of claim 1, wherein the agent comprises an agent that activates eEF2 kinase (eEF2K).

6. The method of claim 1, wherein the agent comprises an agent that inhibits a mitogenic activator of protein kinase (MAPK) pathway.

7. The method of claim 1, wherein the agent comprises an agent that inhibits or suppresses the activity or expression of RASGRP1.

8. The method of claim 1, further comprising administering to the subject a therapeutically effective amount of at least one additional cancer therapy.

9. The method of claim 8, wherein the at least one additional cancer therapy comprises administering to the subject a therapeutically effective amount of a chemotherapeutic agent.

10. The method of claim 8, wherein the agent and the chemotherapeutic agent comprise individually a small molecule, a protein, a fusion protein, a peptide, a nucleic acid, an aptamer, an avimer, or a derivative or fragment thereof.

11. The method of claim 1, further comprising determining the presence of a SWI/SNF chromatin remodeling complex mutation in cells of the subject prior to administering the agent.

12. The method of claim 1, further comprising determining the presence of an ARID1A mutation in a cell of the subject prior to administering the agent.

13. The method of claim 1, wherein the tumor or cancer is selected from a cancer of the brain, breast, bladder, bone, cartilage, cervix, colon, cornea, eye, neural tissue, glia, esophagus, fallopian tube, heart, pancreas, intestines, gallbladder, kidney, liver, lung, ovary, pancreas, parathyroid, pineal gland, pituitary gland, prostate, spinal cord, spleen, skeletal muscle, skin, muscle, stomach, testis, thymus, thyroid, trachea, urogenital tract, ureter, urethra, uterus, endometrium, vagina, or combination thereof.

14. The method of claim 1, wherein the SWI/SNF chromatin remodeling complex mutant, or the ARID1A-deficient tumor or cancer is a urothelial cancer.

15. A method of selecting and/or deselecting a subject having a tumor or cancer for treatment with a therapeutic agent, the method comprising:
(i) obtaining a biological specimen from the tumor of the subject;
(ii) determining the absence or presence of an SWI/SNF chromatin remodeling complex mutation and/or an ARID1A deficiency in the biological specimen obtained from the subject; and
(iii) administering an effective amount of the therapeutic agent to the subject determined to have the SWI/SNF chromatin remodeling complex mutation and/or the ARID1A-deficiency.

16. The method of claim 15, wherein the therapeutic agent comprises an agent effective in preventing, suppressing, or inhibiting mRNA translation elongation.

17. The method of claim 15, wherein the therapeutic agent comprises an agent effective in suppressing or inhibiting eukaryotic elongation factor 2 (eEF2).

18. The method of claim 15, wherein the therapeutic agent comprises an agent effective in inhibiting the activity of eukaryotic elongation factor 2 (eEF2).

19. The method of claim 15, wherein the therapeutic agent comprises an agent that activates eEF2 kinase (eEF2K).

20. The method of claim 15, wherein the therapeutic agent comprises an agent that inhibits a mitogenic activator of a protein kinase (MAPK) pathway.

21. The method of claim 15, wherein the therapeutic agent comprises an agent effective in inhibiting or suppressing the activity or expression of RASGRP1.

22. The method of claim 15, wherein the therapeutic agent comprises an agent effective in inhibiting or suppressing the expression of RASGRP1.

23. The method of claim 15, further comprising administering to the subject a therapeutically effective amount of at least one additional cancer therapy.

24. The method of claim 23, wherein the at least one additional cancer therapy comprises administering to the subject a therapeutically effective amount of a chemotherapeutic agent.

25. The method of claim 24, wherein the therapeutic agent and the chemotherapeutic agent are individually a small molecule, a protein, a fusion protein, a peptide, a nucleic acid, an aptamer, an avimer, or a derivative or fragment thereof.

26. The method of claim 15, wherein the tumor or cancer is a urothelial cancer.

27. A method of preventing uncontrolled cell growth and cancer progression of an ARID1A-deficient tumor or cancer comprising contacting the tumor or cancer with an agent effective in increasing phosphorylation of eEF2 or decreasing eEF2 expression levels.

28. The method of claim 27, wherein the agent effective in increasing phosphorylation of eEF2 comprises an agent that activates eEF2 kinase (eEF2K).

29. The method of claim 27, wherein the agent comprises an agent that inhibits a mitogenic activator of a protein kinase (MAPK) pathway.

30. The method of claim 27, wherein the agent comprises an agent that inhibits or suppresses the expression of RASGRP1.

31. A method for inducing a transcription-translation conflict in an ARID1A-deficient tumor cell of a subject, the method comprising contacting the ARID1A-deficient tumor cell of the subject with an agent effective in preventing, inhibiting, or suppressing mRNA translation elongation.

32. The method of claim 31, wherein the agent effective in inhibiting or suppressing mRNA translation elongation comprises an inhibitor of eEF2 activity or gene expression.

33. The method of claim 31, wherein the agent effective in preventing, inhibiting, or suppressing mRNA translation elongation comprises an agent that suppresses or inhibits eEF2 activity.

34. The method of claim 31, wherein the agent that suppresses or inhibits eEF2 activity is selected from an agent effective in suppressing or inhibiting eEF2 activity, activating eEF2K, suppressing or inhibiting a MAPK pathway, or inhibiting RASGRP1 expression.

35. The method of claim 31, wherein the ARID1A-deficient tumor cell is a urothelial tumor cell.


SEQUENCE LISTING (20 sequences)

SEQ ID NO: 1 - DNA, 24 bp, synthetic construct
  source 1..24: mol_type = other DNA
gtaatgggaaagcgactactggag

SEQ ID NO: 2 - DNA, 21 bp, synthetic construct
  source 1..21: mol_type = other DNA
tgttcgtttttgtggcgggag

SEQ ID NO: 3 - DNA, 23 bp, synthetic construct
  source 1..23: mol_type = other DNA
gcggtctggcagtaaaaactatc

SEQ ID NO: 4 - DNA, 23 bp, synthetic construct
  source 1..23: mol_type = other DNA
gtgaaacagcattgctgtcactt

SEQ ID NO: 5 - DNA, 24 bp, synthetic construct
  source 1..24: mol_type = other DNA
ctaggccacagaattgaaagatct

SEQ ID NO: 6 - DNA, 25 bp, synthetic construct
  source 1..25: mol_type = other DNA
gtaggtggaaattctagcatcatcc

SEQ ID NO: 7 - DNA, 17 bp, synthetic construct
  source 1..17: mol_type = other DNA
ctggcttctgaggaccg

SEQ ID NO: 8 - DNA, 18 bp, synthetic construct
  source 1..18: mol_type = other DNA
caggacaacgcccacaca

SEQ ID NO: 9 - DNA, 18 bp, synthetic construct
  source 1..18: mol_type = other DNA
agggcgaggagctgttca

SEQ ID NO: 10 - DNA, 19 bp, synthetic construct
  source 1..19: mol_type = other DNA
tgaagtcgatgcccttcag

SEQ ID NO: 11 - DNA, 19 bp, synthetic construct
  source 1..19: mol_type = other DNA
gcaagaccctggtcctcac

SEQ ID NO: 12 - DNA, 21 bp, synthetic construct
  source 1..21: mol_type = other DNA
ggaggaagtcagaaccaggac

SEQ ID NO: 13 - DNA, 19 bp, synthetic construct
  source 1..19: mol_type = other DNA
gcaagaccctggtcctcac

SEQ ID NO: 14 - DNA, 18 bp, synthetic construct
  source 1..18: mol_type = other DNA
accggccttattccaagc

SEQ ID NO: 15 - DNA, 21 bp, synthetic construct
  source 1..21: mol_type = other DNA
ggccggctgctagagagtgtc

SEQ ID NO: 16 - DNA, 24 bp, synthetic construct
  source 1..24: mol_type = other DNA
catcagctgattgtagtggacatc

SEQ ID NO: 17 - DNA, 27 bp, synthetic construct
  source 1..27: mol_type = other DNA
tgcgaggccagaggccacttgtgtagc

SEQ ID NO: 18 - DNA, 24 bp, synthetic construct
  source 1..24: mol_type = other DNA
cagggcctgctttcttggtggcag

SEQ ID NO: 19 - DNA, 20 bp, synthetic construct
  source 1..20: mol_type = other DNA
cttcgaatcctgcccccatt

SEQ ID NO: 20 - DNA, 20 bp, synthetic construct
  source 1..20: mol_type = other DNA
tcttgggctgggagagatga